(19) United States
(12) Patent Application Publication
(10) Pub. No.: US 2023/0076527 A1
(43) Pub. Date: Mar. 9, 2023

(54) TREATMENT OF PAROXYSMAL NOCTURNAL HEMOGLOBINURIA

(71) Applicant: Apellis Pharmaceuticals, Inc., Waltham, MA (US)

(72) Inventors: Pascal Deschatelets, West Newton, MA (US); Cedric Francois, Prospect, KY (US); Federico Grossi, Needham, MA (US); Sharon Morriss, Watertown, MA (US); Elizabeth F. Tan, Cambridge (GB)

(21) Appl. No.: 17/791,194

(22) PCT Filed: Jan. 7, 2021
(86) PCT No.: PCT/US2021/012561

§ 371 Date: Jul. 6, 2022

Related U.S. Application Data
(60) Provisional application No. 63/124,006, filed on Dec. 10, 2020.
(60) Provisional application No. 63/080,648, filed on Sep. 18, 2020.
(60) Provisional application No. 63/038,607, filed on Jun. 12, 2020.
(60) Provisional application No. 62/961,032, filed on Jan. 14, 2020.
(60) Provisional application No. 62/958,265, filed on Jan. 7, 2020.

(51) Int. Cl. A61K 47/60; A61K 47/54; A61K 47/64; A61K 39/395; A61P 7/06

(52) U.S. Cl. CPC A61K 47/60; A61K 39/3955; A61K 47/64; A61K 47/542; A61P 7/06; A61K 2039/545

(57) ABSTRACT

Methods and compositions for treatment of paroxysmal nocturnal hemoglobinuria are described. In some aspects, the disclosure features a method of treating a subject suffering from paroxysmal nocturnal hemoglobinuria (PNH), comprising subcuta-neously administering to the subject pegcetacoplan.

### CROSS REFERENCE TO RELATED APPLICATIONS

This application claims the benefit of U.S. Provisional Application No. 62/958,265, filed Jan. 7, 2020, U.S. Provisional Application No. 62/961,032, filed Jan. 14, 2020, U.S. Provisional Application No. 63/038,607, filed Jun. 12, 2020, U.S. Provisional Application No. 63/080,648, filed Sep. 18, 2020, and U.S. Provisional Application No. 63/124,006, filed Dec. 10, 2020, the contents of all of which are hereby incorporated herein in their entirety.

### BACKGROUND

An eculizumab drug (Soliris®) is approved in the U.S. for the treatment of paroxysmal nocturnal hemoglobinuria (PNH). However, there remains a need for effective therapy for PNH.

### SUMMARY

In some aspects, the disclosure features a method of treating a subject suffering from paroxysmal nocturnal hemoglobinuria (PNH), comprising subcutaneously administering to the subject pegcetacoplan, wherein if the subject’s LDH level is less than or equal to twice the upper limit of normal, pegcetacoplan is administered in a 1080 mg dose twice weekly, and/or if the subject’s LDH level is greater than twice the upper limit of normal, pegcetacoplan is administered in a 1080 mg dose every three days. In some embodiments, the subject initially is administered pegcetacoplan in a 1080 mg dose twice weekly, and if during the treatment, the subject’s LDH level is assessed to be greater than twice the upper limit of normal, the subject subsequently is administered pegcetacoplan in a 1080 mg dose every three days. In some embodiments, if the subject is administered pegcetacoplan in a 1080 mg dose every three days after exhibiting an LDH level greater than twice the upper limit of normal, the method further comprises having the subject’s LDH level assessed twice weekly for at least two weeks. In some embodiments, the upper limit of normal is about 225 U/L, e.g., in some embodiments, is 225 U/L.

In some aspects, the disclosure features a method of treating a subject suffering from PNH, comprising subcutaneously administering to the subject pegcetacoplan, wherein the patient is treated with a C5 inhibitor at a current dose prior to administration of a first dose of pegcetacoplan, and wherein (a) during the first four weeks of treatment, pegcetacoplan is administered in a 1080 mg dose twice weekly or every three days and the C5 inhibitor is administered at the current dose, and (b) after the first four weeks of treatment, pegcetacoplan is administered in a 1080 mg dose twice weekly or every three days and the administration of the C5 inhibitor is discontinued. For example, in some embodiments, during the first four weeks of treatment, pegcetacoplan is subcutaneously administered to the subject in a 1080 mg dose twice weekly, and the C5 inhibitor is administered at the current dose, and after the first four weeks of treatment, pegcetacoplan is subcutaneously administered to the subject in a 1080 mg dose twice weekly and the administration of the C5 inhibitor is discontinued. In some embodiments, during the first four weeks of treatment, pegcetacoplan is subcutaneously administered to the subject in a 1080 mg dose every three days, and the C5 inhibitor is administered at the current dose, and after the first four weeks of treatment, pegcetacoplan is subcutaneously administered to the subject in a 1080 mg dose every three days and the administration of the C5 inhibitor is discontinued. In some embodiments, the dosing frequency is changed from twice weekly to every three days, and/or from every three days to twice weekly, e.g., accordingly to the subject’s LDH level. In some embodiments, if the subject’s LDH level is less than or equal to twice the upper limit of normal, pegcetacoplan is administered in a 1080 mg dose twice weekly and if the subject’s LDH level is greater than twice the upper limit of normal, pegcetacoplan is administered in a 1080 mg dose every three days. In some embodiments, the subject initially is administered pegcetacoplan in a 1080 mg dose twice weekly, and if during the treatment, the subject’s LDH level is assessed to be greater than twice the upper limit of normal, the subject subsequently is administered pegcetacoplan in a 1080 mg dose every three days. In some embodiments, if the subject is administered pegcetacoplan in a 1080 mg dose every three days after exhibiting an LDH level greater than twice the upper limit of normal, the method further comprises having the subject’s LDH level assessed twice weekly for at least two weeks. In some embodiments, the upper limit of normal is about 225 U/L, e.g., in some embodiments, is 225 U/L. In some embodiments, the subject is transfusion-dependent at the current dose of the C5 inhibitor and before administration of the first dose of pegcetacoplan, and/or the subject’s hemoglobin level is less than about 11 g/dL, less than about 10.5 g/dL, less than about 10 g/dL, less than about 9 g/dL, or less than about 8 g/dL, at the current dose of the C5 inhibitor and before administration of the first dose of pegcetacoplan. In some embodiments, the C5 inhibitor is an anti-C5 antibody. In certain embodiments, the anti-C5 antibody is eculizumab.

In some embodiments, the disclosure features a method of treating a subject suffering from PNH, comprising subcutaneously administering to the subject 1080 mg of pegcetacoplan in a 20 mL solution twice weekly. In some embodiments, the disclosure features a method of treating a subject suffering from PNH, comprising subcutaneously administering to the subject 1080 mg of pegcetacoplan in a 20 mL solution every three days. In some embodiments, if the subject’s LDH level is less than or equal to twice the upper limit of normal, pegcetacoplan is administered in a 1080 mg dose twice weekly and if the subject’s LDH level is greater than twice the upper limit of normal, pegcetacoplan is administered in a 1080 mg dose every three days. In some embodiments, the subject initially is administered pegcetacoplan in a 1080 mg dose twice weekly, and if during the treatment, the subject’s LDH level is assessed to be greater than twice the upper limit of normal, the subject subsequently is administered pegcetacoplan in a 1080 mg dose every three days. In some embodiments, if the subject is administered pegcetacoplan in a 1080 mg dose every three days after exhibiting an LDH level greater than twice the upper limit of normal, the method further comprises having the subject’s LDH level assessed twice weekly for at least two weeks. In some embodiments, the upper limit of normal is about 225 U/L, e.g., in some embodiments, is 225 U/L.

In some aspects, the disclosure features a method of increasing the level of hemoglobin, in a subject suffering from PNH, to a target hemoglobin level, the method comprising subcutaneously administering to the subject about 1080 mg pegcetacoplan twice weekly or every three days, thereby increasing hemoglobin in the subject to the target hemoglobin level.

In some embodiments, the target hemoglobin level is a hemoglobin level that is higher, relative to a control hemoglobin level, by at least about 1 g/dL, e.g., in some embodiments, by at least about 2 g/dL, e.g., by at least 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 g/dL. In some embodiments, the target hemoglobin level is a hemoglobin level that is higher, relative to a control hemoglobin level, by at least about 20%, 40%, 60%, 80%, 100%, or more. In some embodiments, the control hemoglobin level is a hemoglobin level in a subject suffering from PNH and not being administered pegcetacoplan; a hemoglobin level in the subject before administration of pegcetacoplan; or a lower limit of a range of hemoglobin levels in a healthy subject. In some embodiments, the subject suffering from PNH and not being administered pegcetacoplan receives a current dose of a C5 inhibitor, e.g., in some embodiments an anti-C5 antibody, e.g., in some embodiments eculizumab. In some embodiments, the target hemoglobin level is about 10 g/dL to about 15 g/dL. In some embodiments, the target hemoglobin level is about 11 g/dL, about 12 g/dL, or about 13 g/dL, e.g., in some embodiments the target hemoglobin level is about 11 to about 12 g/dL. In some embodiments, the target hemoglobin level is at least 2 g/dL higher, e.g., in some embodiments is about 2.4 g/dL higher, than a hemoglobin level in the subject before administration of pegcetacoplan. In some embodiments, the target hemoglobin level is sustained for at least 16 weeks after the subject’s first dose of pegcetacoplan.

In some embodiments, the method further comprises measuring or having measured hemoglobin level in the subject, e.g., in a biological sample from the subject. In some embodiments, the method comprises measuring or having measured hemoglobin level in the subject before and/or after administration of pegcetacoplan. In some embodiments, hemoglobin is increased in the subject after administration of pegcetacoplan and in the absence of a transfusion.

In some aspects, the disclosure features a method of treating a subject suffering from PNH, the method comprising subcutaneously administering to the subject about 1080 mg pegcetacoplan twice weekly or every three days, wherein the treatment increases hemoglobin in the subject to a target hemoglobin level.

In some embodiments, the target hemoglobin level is a hemoglobin level that is higher, relative to a control hemoglobin level, by at least about 1 g/dL, e.g., in some embodiments, by at least about 2 g/dL, e.g., by at least 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 g/dL. In some embodiments, the target hemoglobin level is a hemoglobin level that is higher, relative to a control hemoglobin level, by at least about 20%, 40%, 60%, 80%, 100%, or more. In some embodiments, the control hemoglobin level is a hemoglobin level in a subject suffering from PNH and not being administered pegcetacoplan; a hemoglobin level in the subject before administration of pegcetacoplan; or a lower limit of a range of hemoglobin levels in a healthy subject. In some embodiments, the subject suffering from PNH and not being administered pegcetacoplan receives a current dose of a C5 inhibitor, e.g., in some embodiments an anti-C5 antibody, e.g., in some embodiments eculizumab. In some embodiments, the target hemoglobin level is about 10 g/dL to about 15 g/dL. In some embodiments, the target hemoglobin level is about 11 g/dL, about 12 g/dL, or about 13 g/dL, e.g., in some embodiments, the target hemoglobin level is about 11 to about 12 g/dL. In some embodiments, the target hemoglobin level is at least 2 g/dL higher, e.g., in some embodiments, is about 2.4 g/dL higher, than a hemoglobin level in the subject before administration of pegcetacoplan. In some embodiments, the target hemoglobin level is sustained for at least 16 weeks after the subject’s first dose of pegcetacoplan.

In some embodiments, the treatment results in the subject’s hemoglobin level increasing to at least 11 g/dL. In some embodiments, the treatment results in the subject’s hemoglobin level increasing at least 2 g/dL from the subject’s hemoglobin level before administration of the first dose of pegcetacoplan.

In some embodiments, the method further comprises measuring or having measured hemoglobin level in the subject. In some embodiments, the method further comprises measuring or having measured hemoglobin level in the subject before and/or after administration of pegcetacoplan. In some embodiments, the subject is treated in the absence of a transfusion.

In some aspects, the disclosure features a method of reducing the number of transfusions administered to a subject to a target number of transfusions, the method comprising subcutaneously administering to the subject about 1080 mg pegcetacoplan twice weekly or every three days, thereby reducing the number of transfusions to the target number of transfusions, and wherein the subject suffers from PNH.

In some embodiments, the target number of transfusions is at least 1 (e.g., at least 2, 3, 4, 5, 6 or more) fewer transfusions over a defined period of time relative to a control number of transfusions. In some embodiments, the control number of transfusions is a number of transfusions administered to a subject suffering from PNH and not being administered pegcetacoplan; or a number of transfusions administered to the subject before administration of pegcetacoplan. In some embodiments, the subject suffering from PNH and not receiving pegcetacoplan receives a current dose of a C5 inhibitor, e.g., in some embodiments an anti-C5 antibody, e.g., in some embodiments eculizumab. In some embodiments, the target number of transfusions is fewer than 3, 2, or 1 transfusions over about 4 weeks, 8 weeks, 12 weeks, 16 weeks, 20 weeks, 24 weeks, or more. In some embodiments, the target number of transfusions is zero transfusions over about 4 weeks, 8 weeks, 12 weeks, 16 weeks, 20 weeks, 24 weeks, or more.

In some embodiments, the method further comprises assessing or having assessed the need for administering a transfusion to the subject. In some embodiments, the method further comprises assessing or having assessed the need for administering a transfusion to the subject before and/or after administration of pegcetacoplan.

In some aspects, the disclosure features a method of reducing the number of packed red blood cell (PRBC) units administered to a subject to a target number of PRBC units, the method comprising subcutaneously administering to the subject about 1080 mg pegcetacoplan twice weekly or every three days, thereby reducing the number of administered PRBC units to the target number of PRBC units, and wherein the subject suffers from PNH.

In some embodiments, the target number of PRBC units is at least 1 (e.g., at least 2, 3, 4, 5, 6 or more) fewer PRBC units administered over a defined period of time relative to a control number of PRBC units. In some embodiments, the control number of PRBC units is a number of PRBC units administered to a subject suffering from PNH and not being administered pegcetacoplan; or a number of PRBC units administered to the subject before administration of pegcetacoplan. In some embodiments, the subject suffering from PNH and not being administered pegcetacoplan receives a current dose of a C5 inhibitor, e.g., in some embodiments an anti-C5 antibody, e.g., in some embodiments eculizumab. In some embodiments, the target number of PRBC units is fewer than 3, 2, or 1 PRBC units over about 4 weeks, 8 weeks, 12 weeks, 16 weeks, 20 weeks, 24 weeks, or more.

In some embodiments, the method further comprises assessing or having assessed the need for administering a PRBC unit to the subject. In some embodiments, the method further comprises assessing or having assessed the need for administering a PRBC unit to the subject before and/or after administration of pegcetacoplan.

In some aspects, the disclosure features a method of treating a subject suffering from PNH, the method comprising subcutaneously administering to the subject about 1080 mg pegcetacoplan twice weekly or every three days, wherein the treatment reduces the number of transfusions administered to the subject to a target number of transfusions.

In some embodiments, the target number of transfusions is at least 1 (e.g., at least 2, 3, 4, 5, 6, etc.) fewer transfusions over a defined period of time relative to a control number of transfusions. In some embodiments, the control number of transfusions is a number of transfusions administered to a subject suffering from PNH and not being administered pegcetacoplan; or a number of transfusions administered to the subject before administration of pegcetacoplan. In some embodiments, the subject suffering from PNH and not being administered pegcetacoplan receives a current dose of a C5 inhibitor, e.g., in some embodiments an anti-C5 antibody, e.g., in some embodiments eculizumab. In some embodiments, the target number of transfusions is fewer than 3, 2, or 1 transfusions over about 4 weeks, 8 weeks, 12 weeks, 16 weeks, 20 weeks, 24 weeks, or more. In some embodiments, the target number of transfusions is zero transfusions over about 4 weeks, 8 weeks, 12 weeks, 16 weeks, 20 weeks, 24 weeks, or more.

In some embodiments, the treatment results in the subject receiving at least one fewer transfusion within 16 weeks following administration of the first dose of pegcetacoplan, compared to the number of transfusions received by the subject before administration of the first dose of pegcetacoplan. In some embodiments, the treatment results in the subject not needing a transfusion for at least 16 weeks following administration of the first dose of pegcetacoplan.

In some embodiments, the method further comprises assessing or having assessed the need for administering a transfusion to the subject. In some embodiments, the method further comprises assessing or having assessed the need for administering a transfusion to the subject before and/or after administration of pegcetacoplan.

In some aspects, the disclosure features a method of treating a subject suffering from PNH, the method comprising subcutaneously administering to the subject about 1080 mg pegcetacoplan twice weekly or every three days, wherein the treatment reduces the number of PRBC units administered to the subject to a target number of PRBC units.

In some embodiments, the target number of PRBC units is at least 1 (e.g., at least 2, 3, 4, 5, 6 or more) fewer PRBC units administered over a defined period of time relative to a control number of PRBC units. In some embodiments, the control number of PRBC units is a number of PRBC units administered to a subject suffering from PNH and not being administered pegcetacoplan; or a number of PRBC units administered to the subject before administration of pegcetacoplan. In some embodiments, the subject suffering from PNH and not being administered pegcetacoplan receives a current dose of a C5 inhibitor, e.g., in some embodiments an anti-C5 antibody, e.g., in some embodiments eculizumab. In some embodiments, the target number of PRBC units is fewer than 3, 2, or 1 PRBC units over about 4 weeks, 8 weeks, 12 weeks, 16 weeks, 20 weeks, 24 weeks, or more.

In some embodiments, the treatment results in the subject receiving at least one fewer PRBC unit within 16 weeks following administration of the first dose of pegcetacoplan, compared to the number of PRBC units received by the subject before administration of the first dose of pegcetacoplan. In some embodiments, the treatment results in the subject receiving 11 or fewer PRBC units within 16 weeks following administration of the first dose of pegcetacoplan.

In some embodiments, the method further comprises assessing or having assessed the need for administering a PRBC unit to the subject. In some embodiments, the method further comprises assessing or having assessed the need for administering a PRBC unit to the subject before and/or after administration of pegcetacoplan.

In some aspects, the disclosure features a method of reducing the number of reticulocytes (i.e., absolute reticulocyte count), in a subject suffering from PNH, to a target reticulocyte level, the method comprising subcutaneously administering to the subject about 1080 mg pegcetacoplan twice weekly or every three days, thereby reducing number of reticulocytes in the subject to the target reticulocyte level.

In some embodiments, the target reticulocyte level is a reticulocyte level that is lower, relative to a control reticulocyte level, by at least about 20%, 40%, 60%, or 80%. In some embodiments, the control reticulocyte level is a reticulocyte level in a subject suffering from PNH and not being administered pegcetacoplan; a reticulocyte level in the subject before administration of pegcetacoplan; or an upper limit of a range of reticulocyte levels in a healthy subject. In some embodiments, the subject suffering from PNH and not being administered pegcetacoplan receives a current dose of a C5 inhibitor, e.g., in some embodiments an anti-C5 antibody, e.g., in some embodiments eculizumab. In some embodiments, the target reticulocyte level is about 30 to about 120 × 10⁹/L. In some embodiments, the target reticulocyte level is about 30 to about 100 × 10⁹ /L, e.g., in some embodiments, about 70, 80, or 90 × 10⁹ /L. In some embodiments, the target reticulocyte level is about 60 to 85 × 10⁹ /L. In some embodiments, the target reticulocyte level is about 70 to 80 × 10⁹ /L. In some embodiments, the target reticulocyte level is about 135 × 10⁹ /L lower than a reticulocyte level in the subject before administration of pegcetacoplan. In some embodiments, the target reticulocyte level is sustained for at least 16 weeks after the subject’s first dose of pegcetacoplan.

In some embodiments, the method further comprises measuring or having measured reticulocyte level in the subject. In some embodiments, the method further comprises measuring or having measured reticulocyte level in the subject before and/or after administration of pegcetacoplan. In some embodiments, the number of reticulocytes is decreased in the subject after administration of pegcetacoplan and in the absence of a transfusion.

In some aspects, the disclosure features a method of treating a subject suffering from PNH, the method comprising subcutaneously administering to the subject about 1080 mg pegcetacoplan twice weekly or every three days, wherein the treatment decreases the number of reticulocytes (e.g., absolute reticulocyte count) in the subject to a target reticulocyte level.

In some embodiments, the target reticulocyte level is a reticulocyte level that is lower, relative to a control reticulocyte level, by at least about 20%, 40%, 60%, or 80%. In some embodiments, the control reticulocyte level is a reticulocyte level in a subject suffering from PNH and not being administered pegcetacoplan; a reticulocyte level in the subject before administration of pegcetacoplan; or an upper limit of a range of reticulocyte levels in a healthy subject. In some embodiments, the subject suffering from PNH and not being administered pegcetacoplan receives a current dose of a C5 inhibitor, e.g., in some embodiments an anti-C5 antibody, e.g., in some embodiments eculizumab. In some embodiments, the target reticulocyte level is about 30 to about 120 × 10⁹ /L. In some embodiments, the target reticulocyte level is about 30 to about 100 × 10⁹ /L, e.g., in some embodiments, about 70, 80, or 90 × 10⁹ /L. In some embodiments, the target reticulocyte level is about 60 to 85 × 10⁹ /L. In some embodiments, the target reticulocyte level is about 70 to 80 × 10⁹ /L. In some embodiments, the target reticulocyte level is about 135 × 10⁹ /L lower than a reticulocyte level in the subject before administration of pegcetacoplan. In some embodiments, the target reticulocyte level is sustained for at least 16 weeks after the subject’s first dose of pegcetacoplan.

In some embodiments, the treatment results in normalization of the subject’s reticulocyte level. In some embodiments, a normalized reticulocyte level is a reticulocyte level of about 30-120 × 10⁹ cells/L. In some embodiments, the treatment results in reducing the subject’s reticulocyte level to 70-80 × 10⁹ cells/L. In some embodiments, the treatment results in the subject’s reticulocyte level decreasing at least 135 × 10⁹ cells/L from the subject’s reticulocyte level before administration of the first dose of pegcetacoplan.

In some embodiments, the method further comprises measuring or having measured reticulocyte level in the subject. In some embodiments, the method further comprises measuring or having measured reticulocyte level in the subject before and/or after administration of pegcetacoplan. In some embodiments, the subject is treated in the absence of a transfusion.

In some aspects, the disclosure features a method of reducing lactate dehydrogenase (LDH) level, in a subject suffering from PNH, to a target LDH level, the method comprising subcutaneously administering to the subject about 1080 mg pegcetacoplan twice weekly or every three days, thereby reducing LDH level in the subject to the target LDH level.

In some embodiments, the target LDH level is a LDH level that is lower, relative to a control LDH level, by at least about 20%, 40%, 60%, or 80%. In some embodiments, the control LDH level is a LDH level in a subject suffering from PNH and not being administered pegcetacoplan; a LDH level in the subject before administration of pegcetacoplan; or an upper limit of a range of LDH levels in a healthy subject. In some embodiments, the subject suffering from PNH and not being administered pegcetacoplan receives a current dose of a C5 inhibitor, e.g., in some embodiments an anti-C5 antibody, e.g., in some embodiments eculizumab. In some embodiments, the target LDH level is about 110 to about 225 U/L, e.g., n some embodiments, iabout 120, 140, 160, 180, 200, or 220 U/L. In some embodiments, the target LDH level is about 160 to 230 U/L. In some embodiments, the target LDH level is about 190 U/L. In some embodiments, the target LDH level is about 15 U/L lower than an LDH level in the subject before administration of pegcetacoplan. In some embodiments, the target LDH level is sustained for at least 16 weeks after the subject’s first dose of pegcetacoplan.

In some embodiments, the method further comprises measuring or having measured LDH level in the subject. In some embodiments, the method further comprises measuring or having measured LDH level in the subject before and/or after administration of pegcetacoplan. In some embodiments, LDH level is decreased in the subject after administration of pegcetacoplan and in the absence of a transfusion.

In some aspects, the disclosure features a method of treating a subject suffering from PNH, the method comprising subcutaneously administering to the subject about 1080 mg pegcetacoplan twice weekly or every three days, wherein the treatment decreases LDH level in the subject to a target LDH level.

In some embodiments, the target LDH level is a LDH level that is lower, relative to a control LDH level, by at least about 20%, 40%, 60%, or 80%. In some embodiments, the control LDH level is a LDH level in a subject suffering from PNH and not being administered pegcetacoplan; a LDH level in the subject before administration of pegcetacoplan; or an upper limit of a range of LDH levels in a healthy subject. In some embodiments, the subject suffering from PNH and not being administered pegcetacoplan receives a current dose of a C5 inhibitor, e.g., in some embodiments an anti-C5 antibody, e.g., in some embodiments eculizumab. In some embodiments, the target LDH level is about 110 to about 225 U/L, e.g., in some embodiments, about 120, 140, 160, 180, 200, or 220 U/L. In some embodiments, the target LDH level is about 160 to 230 U/L. In some embodiments, the target LDH level is about 190 U/L. In some embodiments, the target LDH level is about 15 U/L lower than an LDH level in the subject before administration of pegcetacoplan. In some embodiments, the target LDH level is sustained for at least 16 weeks after the subject’s first dose of pegcetacoplan.

In some embodiments, the treatment results in normalization of the subject’s LDH levels. In some embodiments, a normalized LDH level is an LDH level of about 113-226 U/L. In some embodiments, a normalized LDH level is an LDH level of about 110-225 U/L. In some embodiments, the treatment results in the subject’s LDH level decreasing at least 15 U/L from the subject’s LDH level before administration of the first dose of pegcetacoplan.

In some embodiments, the method further comprises measuring or having measured LDH level in the subject. In some embodiments, the method further comprises measuring or having measured LDH level in the subject before and/or after administration of pegcetacoplan. In some embodiments, the subject is treated in the absence of a transfusion.

In some aspects, the disclosure features a method of reducing fatigue level, in a subject suffering from PNH, to a target fatigue level, the method comprising subcutaneously administering to the subject about 1080 mg pegcetacoplan twice weekly or every three days, thereby reducing fatigue level in the subject to the target fatigue level.

In some embodiments, fatigue level is assessed using a FACIT-fatigue scale score. In some embodiments, the target fatigue level is a FACIT-fatigue scale score that is higher, relative to a control FACIT-fatigue scale score, by at least 5, 10, 15, 20, or more points. In some embodiments, the control FACIT-fatigue scale score is a FACIT-fatigue scale score from a subject suffering from PNH and not being adminstered pegcetacoplan; a FACIT-fatigue scale score from the subject before administration of pegcetacoplan; or a lower limit of a range of FACIT-fatigue scale scores from a healthy subject. In some embodiments, the subject suffering from PNH and not being administered pegcetacoplan receives a current dose of a C5 inhibitor, e.g., in some embodiments an anti-C5 antibody, e.g., in some embodiments eculizumab. In some embodiments, the target fatigue level is a FACIT-fatigue scale score of about 32, 34, 36, 38, 40, 42, 44, 46, or 48. In some embodiments, the target fatigue level is a FACIT-fatigue scale score of about 40 to about 44. In some embodiments, the target fatigue level is a FACIT-fatigue scale score that is about 7.5 to about 11 points higher, e.g., in some embodiments is about 9 points higher, than a FACIT-fatigue scale score from the subject before administration of pegcetacoplan. In some embodiments, the target fatigue level is sustained for at least 16 weeks after the subject’s first dose of pegcetacoplan.

In some embodiments, the method further comprises assessing FACIT-fatigue scale score from the subject. In some embodiments, the method further comprises assessing FACIT-fatigue scale score from the subject before and/or after administration of pegcetacoplan. In some embodiments, FACIT-fatigue scale score from the subject is increased after administration of pegcetacoplan and in the absence of a transfusion.

In some aspects, the disclosure features a method of treating a subject suffering from PNH, the method comprising subcutaneously administering to the subject about 1080 mg pegcetacoplan twice weekly or every three days, wherein the treatment reduces fatigue level in the subject to a target fatigue level.

In some embodiments, fatigue level is assessed using a FACIT-fatigue scale score. In some embodiments, the target fatigue level is a FACIT-fatigue scale score that is higher, relative to a control FACIT-fatigue scale score, by at least 5, 10, 15, 20, or more points. In some embodiments, the control FACIT-fatigue scale score is a FACIT-fatigue scale score from a subject suffering from PNH and not being administered pegcetacoplan; a FACIT-fatigue scale score from the subject before administration of pegcetacoplan; or a lower limit of a range of FACIT-fatigue scale scores from a healthy subject. In some embodiments, the subject suffering from PNH and not being administered pegcetacoplan receives a current dose of a C5 inhibitor, e.g., in some embodiments an anti-C5 antibody, e.g., in some embodiments eculizumab. In some embodiments, the target fatigue level is a FACIT-fatigue scale score of about 32, 34, 36, 38, 40, 42, 44, 46, or 48. In some embodiments, the target fatigue level is a FACIT-fatigue scale score of about 40 to about 44. In some embodiments, the target fatigue level is a FACIT-fatigue scale score that is about 7.5 to about 11 points higher, e.g., in some embodiments is about 9 points higher, than a FACIT-fatigue scale score from the subject before administration of pegcetacoplan. In some embodiments, the target fatigue level is sustained for at least 16 weeks after the subject’s first dose of pegcetacoplan.

In some embodiments, the treatment results in the subject’s FACIT-fatigue scale score increasing to at least 40. In some embodiments, the treatment results in the subject’s FACIT-fatigue scale score increasing at least 9 points from the subject’s FACIT-fatigue scale score before administration of the first dose of pegcetacoplan.

In some embodiments, the method further comprises assessing FACIT-fatigue scale score from the subject. In some embodiments, the method further comprises assessing FACIT-fatigue scale score from the subject before and/or after administration of pegcetacoplan. In some embodiments, the subject is treated in the absence of a transfusion.

In some aspects described herein, prior to administration of pegcetacoplan, the subject has not received a C5 inhibitor, e.g., in some embodiments an anti-C5 antibody, e.g., in some embodiments eculizumab.

In some aspects described herein, prior to administration of pegcetacoplan, the subject has received a C5 inhibitor, e.g., in some embodiments an anti-C5 antibody, e.g., in some embodiments eculizumab. In some embodiments, the subject remains transfusion-dependent after receiving the C5 inhibitor, e.g., in some embodiments the anti-C5 antibody, e.g., in some embodiments eculizumab. In some embodiments, the subject has a hemoglobin level of less than about 12 g/dL, less than about 11 g/dL, less than about 10.5 g/dL, less than about 10 g/dL, less than about 9 g/dL, or less than about 8 g/dL after receiving the C5 inhibitor, e.g., in some embodiments the anti-C5 antibody, e.g., in some embodiments eculizumab. In some embodiments, the subject receives at least one dose of pegcetacoplan in combination with at least one dose of a C5 inhibitor, e.g., in some embodiments an anti-C5 antibody, e.g., in some embodiments eculizumab.

In some aspects described herein, after administration of pegcetacoplan, the subject does not receive a dose of a C5 inhibitor, e.g., in some embodiments an anti-C5 antibody, e.g., in some embodiments eculizumab.

In some aspects described herein, pegcetacoplan is administered for at least about 12 weeks, about 16 weeks, about 20 weeks, about 24 weeks, about 28 weeks, about 32 weeks, about 36 weeks, about 40 weeks, about 44 weeks, about 48 weeks, or at least about 52 weeks.

In some aspects described herein, about 1080 mg pegcetacoplan is self-administered twice weekly or every three days using a pump.

In some aspects, the disclosure features a method of treating a subject suffering from PNH, the method comprising subcutaneously administering to the subject about 1080 mg pegcetacoplan twice weekly or every three days. In some embodiments, pegcetacoplan, is administered as a solution in 5% dextrose, as a solution in acetate-buffered mannitol, or as a solution in acetate-buffered sorbitol for subcutaneous administration, e.g., for self-administration subcutaneously. In some embodiments, pegcetacoplan is administered as a sterile solution of in acetate-buffered sorbitol with a pH of about 5.0, weakly buffered, with an osmolality of between 250 and 350 mOsm/kg. In some embodiments, pegcetacoplan is administered using a commercially available pump suitable for subcutaneous infusion.

In some embodiments, the subject is a human subject.

### BRIEF DESCRIPTION OF THE DRAWING

FIG. 1A shows the structure of pegcetacopan (“APL-2”), assuming n of about 800 to about 1100 and a PEG of about 40 kD.

FIG. 1B is a schematic of the PEGASUS trial design.

FIG. 2A shows change in hemoglobin (g/dL) from baseline to week 16 (MMRM Analysis) for the APL-2 group and the eculizumab group.

FIG. 2B shows hemoglobin (g/dL) levels from baseline to week 16 (observed data over time) for the APL-2 group and the eculizumab group.

FIG. 2C shows adjusted change from baseline at week 16 in hemoglobin levels, stratified by transfusion history and platelet count (LS = least squares; CI = confidence interval; *Values are change from baseline at week 12; no transfusion-free patients with uncensored data remained in this stratum in the eculizumab group at week 16; MMRM model excludes post transfusion data for patients with transfusion).

FIG. 2D shows hemoglobin levels from baseline to week 16 (all available data in all patients regardless of transfusion events) for the APL-2 group and the eculizumab group.

FIG. 3A shows transfusion avoidance for the APL-2 group and the eculizumab group.

FIG. 3B shows effect of pegcetacoplan on transfusion avoidance (overall and transfusion strata).

FIG. 3C shows number of PRBC units transfused for the APL-2 group and the eculizumab group.

FIG. 4A shows change in absolute reticulocyte count (10⁹ /L) from baseline to week 16 (MMRM Analysis) for the APL-2 group and the eculizumab group.

FIG. 4B shows absolute reticulocyte count (10⁹ /L) from baseline to week 16 (observed data over time) for the APL-2 group and the eculizumab group.

FIG. 5A shows change in LDH (U/L) from baseline to week 16 (MMRM Analysis) for the APL-2 group and the eculizumab group.

FIG. 5B shows LDH (U/L) levels from baseline to week 16 (observed data over time) for the APL-2 group and the eculizumab group.

FIG. 5C shows indirect bilirubin levels from baseline to week 16 (including post-transfusion data) for the APL-2 group and the eculizumab group.

FIG. 6A shows change in FACIT-fatigue score from baseline to week 16 (MMRM Analysis) for the APL-2 group and the eculizumab group.

FIG. 6B shows FACIT-fatigue scores from baseline to week 16 (observed data over time) for the APL-2 group and the eculizumab group.

FIG. 6C shows correlation of FACIT-fatigue total score with hemoglobin at week 16 for the APL-2 group and the eculizumab group.

FIG. 6D shows correlation of change in FACIT-fatigue total score with change in hemoglobin from Day 1 to week 16 for the APL-2 group and the eculizumab group.

FIG. 7A shows a summary of analysis of primary and key secondary endpoints. LDH = Lactate Dehydrogenase. FACIT = Functional Assessment of Chronic Illness Therapy. Mean (SE) = Adjusted means (SE) are based on the mixed model repeated measures (MMRM) analysis. CI = Confidence Interval. SE = Standard Error. Key Secondary Endpoints analyses were based on pre-specified Non-Inferiority Margins. Non-inferiority was achieved if the LCL or UCL of the 95% CI of the treatment difference met the pre-specified margin. * Not Tested: As LDH did not achieve non-inferiority, no other endpoints were tested. NRR denotes normal reference range.

FIG. 7B shows a summary of analysis of key secondary endpoints (including post-transfusion data). FACIT, Functional Assessment of Chronic Illness Therapy; LDH, lactate dehydrogenase; LS, least square; MMRM, mixed model repeated measures; NRR, normal reference range; Mean (SE), adjusted means (SE) are based on MMRM analysis. Key secondary endpoint analyses are based on pre-specified non-inferiority margins. Difference is adjusted for strata.

FIG. 8 shows normalization of hematologic markers and clinically meaningful improvement on FACIT-fatigue score at 16 weeks. Hemoglobin normal range: females ≥ 12 to 16 g/dL, males ≥13.6-18 g/dL. Reticulocyte normalization: 30-120 × 10⁹ cells/L. LDH normal range: 113-226 U/L.

FIG. 9A shows C3d loading on red blood cells in a single patient randomized to eculizumab and in a single patient randomized to pegcetacoplan.

FIG. 9B shows level of C3 deposition on Type III RBCs in pegcetacoplan or eculizumab subjects.

FIG. 9C shows clone size and C3 loading for pegcetacoplan and eculizumab subjects.

FIG. 10 shows hematologic responses in pegcetacoplan and eculizumab-treated subjects at week 16.

FIG. 11 shows anchored comparisons of select endpoints related to hemoglobin and fatigue through Week 16 (PEGASUS study) and Week 26 (302 study) after matching.

FIG. 12 shows mean hemoglobin (g/dL) levels from baseline to week 48 (observed data over time) for the APL-2 group and the eculizumab group. After the 16-week randomized control period, all patients (the APL-2 group and the eculizumab group) entered the open-label period and received APL-2 from Week 17 to Week 48.

### DEFINITIONS

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, a genetically-engineered animal, and/or a clone.

Antibody: As used herein, the term “antibody” refers to an immunoglobulin or a derivative thereof containing an immunoglobulin domain capable of binding to an antigen. The antibody can be of any species, e.g., human, rodent, rabbit, goat, chicken, etc. The antibody may be a member of any immunoglobulin class, including any of the human classes: IgG, IgM, IgA, IgD, and IgE, or subclasses thereof such as IgG1, IgG2, etc. In various embodiments of the disclosure the antibody is a fragment such as a Fabʹ, F(ab')₂, scFv (single-chain variable) or other fragment that retains an antigen binding site, or a recombinantly produced scFv fragment, including recombinantly produced fragments. See, e.g., Allen, T., Nature Reviews Cancer, Vol.2, 750-765, 2002, and references therein. The antibody can be monovalent, bivalent or multivalent. The antibody may be a chimeric or “humanized” antibody in which, for example, a variable domain of rodent origin is fused to a constant domain of human origin, thus retaining the specificity of the rodent antibody. The domain of human origin need not originate directly from a human in the sense that it is first synthesized in a human being. Instead, “human” domains may be generated in rodents whose genome incorporates human immunoglobulin genes. See, e.g., Vaughan, et al., (1998), Nature Biotechnology, 16: 535-539. The antibody may be partially or completely humanized. An antibody may be polyclonal or monoclonal, though for purposes of the present disclosure monoclonal antibodies are generally preferred. Methods for producing antibodies that specifically bind to virtually any molecule of interest are known in the art. For example, monoclonal or polyclonal antibodies can be purified from blood or ascites fluid of an animal that produces the antibody (e.g., following natural exposure to or immunization with the molecule or an antigenic fragment thereof), can be produced using recombinant techniques in cell culture or transgenic organisms, or can be made at least in part by chemical synthesis.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). In some embodiments, the term “about X” includes the number “X” and numbers that fall within a range of 5%, 10%, 15%, or 20% in either direction (greater than or less than) of the number X.

Combination therapy: The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered followed by one or more additional agents. In the separate administration protocol, two or more agents may be administered a few minutes apart, or a few hours apart, or a few days apart, or a few weeks apart. In some embodiments, two or more agents may be administered 1-2 weeks apart. In some embodiments, if two or more agents useful for treating the same disease are administered in combination, each of the two or more agents may be administered using a dosing regimen that would be used if such agent were being used as the sole agent for treating the disease. For example, in some embodiments, if two or more complement inhibitors useful for treating the same disease, e.g., PNH, are administered in combination, each of the two or more agents may be administered using a dosing regimen that would be used if such complement inhibitor were being used as the sole agent for treating the disease.

Complement component: As used herein, the terms “complement component” or “complement protein” is a molecule that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, H, and I, and properdin, with factor H being a negative regulator of the pathway. Components of the lectin pathway include, e.g., MBL2, MASP-1, and MASP-2. Complement components also include cell-bound receptors for soluble complement components. Such receptors include, e.g., C5a receptor (C5aR), C3a receptor (C3aR), Complement Receptor 1 (CR1), Complement Receptor 2 (CR2), Complement Receptor 3 (CR3), etc. It will be appreciated that the term “complement component” is not intended to include those molecules and molecular structures that serve as “triggers” for complement activation, e.g., antigen-antibody complexes, foreign structures found on microbial or artificial surfaces, etc.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Linked: As used herein, the term “linked”, when used with respect to two or more moieties, means that the moieties are physically associated or connected with one another to form a molecular structure that is sufficiently stable so that the moieties remain associated under the conditions in which the linkage is formed and, preferably, under the conditions in which the new molecular structure is used, e.g., physiological conditions. In certain preferred embodiments of the disclosure the linkage is a covalent linkage. In other embodiments the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linker” or “linking moiety” or “linking portion”, the linkage between the two linked moieties is indirect, and typically each of the linked moieties is covalently bonded to the linker. The linker can be any suitable moiety that reacts with the two moieties to be linked within a reasonable period of time, under conditions consistent with stability of the moieties (which may be protected as appropriate, depending upon the conditions), and in sufficient amount, to produce a reasonable yield.

Local administration: As used herein, the term “local administration” or “local delivery”, in reference to delivery of a complement inhibitor described herein, refers to delivery that does not rely upon transport of the complement inhibitor to its intended target tissue or site via the vascular system. The complement inhibitor described herein may be delivered directly to its intended target tissue or site, or in the vicinity thereof, e.g., in close proximity to the intended target tissue or site. For example, the complement inhibitor may be delivered by injection or implantation of the composition or agent or by injection or implantation of a device containing the composition or agent. Following local administration in the vicinity of a target tissue or site, the complement inhibitor described herein, or one or more components thereof, may diffuse to the intended target tissue or site. It will be understood that once having been locally delivered a fraction of a complement inhibitor described herein (typically only a minor fraction of the administered dose) may enter the vascular system and be transported to another location, including back to its intended target tissue or site. As used herein, the term “local administration” or “local delivery”, in reference to delivery of a viral vector described herein, refers to delivery that can rely upon transport of the viral vector to its intended target tissue or site via the vascular system.

Pharmaceutical composition: As used herein, the term “pharmaceutical composition” refers to a composition in which an active agent is formulated together with one or more pharmaceutically acceptable carriers. In some embodiments, the active agent is present in unit dose amount appropriate for administration in a therapeutic regimen that shows a statistically significant probability of achieving a predetermined therapeutic effect when administered to a relevant population. In some embodiments, a pharmaceutical composition may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Subject: As used herein, the term “subject” or “test subject” refers to any organism to which a provided compound or composition is administered in accordance with the present disclosure e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual or subject who is “suffering from” a disease, disorder, and/or condition has been diagnosed with and/or displays one or more symptoms of a disease, disorder, and/or condition.

Systemic: As used herein, the term “systemic,” in reference to complement components, refers to complement proteins that are synthesized by liver hepatocytes and enter the bloodstream, or are synthesized by circulating macrophages or monocytes or other cells and secreted into the bloodstream.

Systemic complement activation: As used herein, the term “systemic complement activation” is complement activation that occurs in the blood, plasma, or serum and/or involves activation of systemic complement proteins at many locations throughout the body, affecting many body tissues, systems, or organs.

Systemic administration: As used herein, the term “systemic administration” and like terms are used herein consistently with their usage in the art to refer to administration of an agent such that the agent becomes widely distributed in the body in significant amounts and has a biological effect, e.g., its desired effect, in the blood and/or reaches its desired site of action via the vascular system. Typical systemic routes of administration include administration by (i) introducing the agent directly into the vascular system or (ii) subcutaneous, oral, pulmonary, or intramuscular administration wherein the agent is absorbed, enters the vascular system, and is carried to one or more desired site(s) of action via the blood.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent can be an agent that, when administered to a subject, can prevent an undesired side effect, such as an immune response to a viral vector described herein. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or signs of the disease, disorder, and/or condition. In some embodiments, a therapeutically effective amount is administered in a single dose; in some embodiments, multiple unit doses are required to deliver a therapeutically effective amount.

Treating: As used herein, the term “treating” refers to providing treatment, i.e., providing any type of medical or surgical management of a subject. The treatment can be provided in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. “Prevent” refers to causing a disease, disorder, condition, or symptom or manifestation of such not to occur for at least a period of time in at least some individuals. Treating can include administering an agent to the subject following the development of one or more symptoms or manifestations indicative of a complement-mediated condition, e.g., PNH, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition of the disclosure can be administered prophylactically, i.e., before development of any symptom or manifestation of the condition. Typically in this case the subject will be at risk of developing the condition.

### DETAILED DESCRIPTION OF CERTAIN EMBODIMENTS

The disclosure provides methods of treating paroxysmal nocturnal hemoglobinuria (PNH) using a compstatin analog described herein. PNH is an acquired, rare, clonal, non-malignant hematologic disease characterized by complement-mediated red blood cell (RBC) hemolysis with or without hemoglobinuria, an increased susceptibility to thrombotic episodes, and/or some degree of bone marrow dysfunction. The onset of PNH is often insidious. Although there have been reports of spontaneous remission, the course of the disease is generally chronic progressive.

It has been known for many years that PNH is caused by complement-mediated lysis of erythrocyte clones lacking functional CD55 and CD59 on their surface to protect them against this process. As such, these erythrocytes are particularly susceptible to the membrane attack complex (MAC) and have been shown to lyse readily in the presence of complement activation.

Eculizumab is a monoclonal anti-C5 antibody that inhibits the formation of the MAC, and an eculizumab drug (Soliris®) has been approved for the treatment of PNH. However, inhibition of MAC formation does not appear to be sufficient to fully control the disease, as many PNH patients receiving eculizumab treatment still suffer from anemia, with only roughly 13% of patients being classified as complete responders, i.e., achieving transfusion independence and normal hemoglobin (Hb) levels. For example, in one study, most of the patients (53%) were classified as partial responders with decreased transfusion needs and reduced lactate dehydrogenase (LDH), and 33% of patients were poor responders, with unchanged transfusion needs and persistent symptoms (DeZern et al., Eur J Haematol. 90(1):16-24 (2013)).

Recent studies have suggested that significant opsonization of PNH erythrocytes by C3 fragments is observed in patients receiving eculizumab treatment. This opsonization is believed to cause the removal of erythrocytes by the spleen and the liver, resulting in extravascular hemolysis. Extravascular hemolysis can be significant in a subset of eculizumab-treated PNH patients and is considered to be a principal contributor to the lack of complete eculizumab response in most patients.

Without wishing to be bound by theory, extravascular hemolysis, one of the parameters contributing to the ongoing need for RBC transfusions despite eculizumab therapy, is believed to be mediated by C3b opsonization rather than C5-dependent MAC-mediated intravascular hemolysis (Risitano et al., Blood 113:4094-4100 (2009)). While eculizumab is effective in addressing CD59 deficiency by preventing C5-dependent MAC-mediated hemolysis, PNH cells are also deficient in CD55, which normally accelerates the dissociation of C3-convertase enzymes, inhibiting the production of C3 fragments and subsequent opsonization. As a result, in the setting of eculizumab therapy, surviving PNH RBCs become opsonized with C3b, targeting them for clearance through extravascular hemolysis by macrophages bearing complement receptors in the liver and spleen.

Evidence for C3b-mediated extravascular hemolysis was observed in three patients exhibiting a suboptimal hematologic response to eculizumab and massive C3 RBC binding using ⁵¹Cr-labeled RBCs. Although these subjects were still receiving eculizumab and had normal LDH levels, they demonstrated markedly reduced RBC half-lives (10, 11, and 13 days, with a normal range of 25-35 days) and excess counts on images of the spleen and liver (Risitano et al., Blood 113:4094-4100 (2009)). In contrast, C3b opsonization of RBCs is not observed in PNH patients who have not been treated with eculizumab, presumably because RBCs in these patients are rapidly lysed by MAC (Risitano et al., Blood 113:4094-4100 (2009)). While C5 inhibition has had a dramatic positive impact on the lives of many PNH patients, anti-C5 therapy has also led to the emergence of a subpopulation of PNH patients with persistent extravascular hemolysis and RBC transfusion requirements, despite continuous eculizumab therapy, that appear to result at least in part from C3b opsonization of RBCs.

### I. Complement System

Complement is an arm of the innate immune system that plays an important role in defending the body against infectious agents. The complement system comprises more than 30 serum and cellular proteins that are involved in three major pathways, known as the classical, alternative, and lectin pathways. The classical pathway is usually triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form C3 convertase, which cleaves C3 to form C3a and C3b. Binding of C3b to C3 convertase produces C5 convertase, which cleaves C5 into C5a and C5b. C3a, C4a, and C5a are anaphylotoxins and mediate multiple reactions in the acute inflammatory response. C3a and C5a are also chemotactic factors that attract immune system cells such as neutrophils. It will be understood that the names “C2a” and “C2b” were subsequently reversed in the scientific literature.

The alternative pathway is initiated by and amplified at, e.g., microbial surfaces and various complex polysaccharides. In this pathway, hydrolysis of C3 to C3(H₂O), which occurs spontaneously at a low level, leads to binding of factor B, which is cleaved by factor D, generating a fluid phase C3 convertase that activates complement by cleaving C3 into C3a and C3b. C3b binds to targets such as cell surfaces and forms a complex with factor B, which is later cleaved by factor D, resulting in a C3 convertase. Surface-bound C3 convertases cleave and activate additional C3 molecules, resulting in rapid C3b deposition in close proximity to the site of activation and leading to formation of additional C3 convertase, which in turn generates additional C3b. This process results in a cycle of C3 cleavage and C3 convertase formation that significantly amplifies the response. Cleavage of C3 and binding of another molecule of C3b to the C3 convertase gives rise to a C5 convertase. C3 and C5 convertases of this pathway are regulated by cellular molecules CR1, DAF, MCP, CD59, and fH. The mode of action of these proteins involves either decay accelerating activity (i.e., ability to dissociate convertases), ability to serve as cofactors in the degradation of C3b or C4b by factor I, or both. Normally the presence of complement regulatory proteins on cell surfaces prevents significant complement activation from occurring thereon.

The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC). The MAC inserts itself into target cell membranes and causes cell lysis. Small amounts of MAC on the membrane of cells may have a variety of consequences other than cell death.

The lectin complement pathway is initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase described above. Further details are found, e.g., in Kuby Immunology, 6th ed., 2006; Paul, W.E., Fundamental Immunology, Lippincott Williams & Wilkins; 6th ed., 2008; and Walport MJ., Complement. First of two parts. N Engl J Med., 344(14):1058-66, 2001.

Complement activity is regulated by various mammalian proteins referred to as complement control proteins (CCPs) or regulators of complement activation (RCA) proteins (U.S. Pat. No. 6,897,290). These proteins differ with respect to ligand specificity and mechanism(s) of complement inhibition. They may accelerate the normal decay of convertases and/or function as cofactors for factor I, to enzymatically cleave C3b and/or C4b into smaller fragments. CCPs are characterized by the presence of multiple (typically 4-56) homologous motifs known as short consensus repeats (SCR), complement control protein (CCP) modules, or SUSHI domains, about 50-70 amino acids in length that contain a conserved motif including four disulfide-bonded cysteines (two disulfide bonds), proline, tryptophan, and many hydrophobic residues. The CCP family includes complement receptor type 1 (CR1; C3b:C4b receptor), complement receptor type 2 (CR2), membrane cofactor protein (MCP; CD46), decay-accelerating factor (DAF, also known as CD55), complement factor H (fH), and C4b-binding protein (C4bp). CD59 is a membrane-bound complement regulatory protein unrelated structurally to the CCPs. Complement regulatory proteins normally serve to limit complement activation that might otherwise occur on cells and tissues of the mammalian, e.g., human host. Thus, “self” cells are normally protected from the deleterious effects that would otherwise ensue were complement activation to proceed on these cells. Deficiencies or defects in complement regulatory protein(s) are involved in the pathogenesis of a variety of complement-mediated disorders.

### II. Compstatin Analogs

Methods of the disclosure include treatment of PNH using compstatin analogs. Compstatin is a cyclic peptide that binds to C3 and inhibits complement activation. U.S. Pat. No. 6,319,897 describes a peptide having the sequence Ile-[Cys-Val-Val-Gln-Asp-Trp-Gly-His-His-Arg-Cys]-Thr (SEQ ID NO:1), with the disulfide bond between the two cysteines denoted by brackets. It will be understood that the name “compstatin” was not used in U.S. Pat. No. 6,319,897 but was subsequently adopted in the scientific and patent literature (see, e.g., Morikis, et al., Protein Sci., 7(3):619-27, 1998) to refer to a peptide having the same sequence as SEQ ID NO: 2 disclosed in U.S. Pat. No. 6,319,897, but amidated at the C terminus as shown in Table 1 (SEQ ID NO: 8). The term “compstatin” is used herein consistently with such usage (i.e., to refer to SEQ ID NO: 8). Compstatin analogs that have higher complement inhibiting activity than compstatin have been developed. See, e.g., WO2004/026328 (PCT/US2003/029653), Morikis, D., et al., Biochem Soc Trans. 32(Pt 1):28-32, 2004, Mallik, B., et al., J. Med. Chem., 274-286, 2005; Katragadda, M., et al. J. Med. Chem., 49: 4616-4622, 2006; WO2007062249 (PCT/US2006/045539); WO2007044668 (PCT/US2006/039397), WO/2009/046198 (PCT/US2008/078593); WO/2010/127336 (PCT/US2010/033345) and discussion below.

As used herein, the term “compstatin analog” includes compstatin and any complement inhibiting analog thereof. The term “compstatin analog” encompasses compstatin and other compounds designed or identified based on compstatin and whose complement inhibiting activity is at least 50% as great as that of compstatin as measured, e.g., using any complement activation assay accepted in the art or substantially similar or equivalent assays. Certain suitable assays are described in U.S. Pat. No. 6,319,897, WO2004/026328, Morikis, supra, Mallik, supra, Katragadda 2006, supra, WO2007062249 (PCT/US2006/045539); WO2007044668 (PCT/US2006/039397), WO/2009/046198 (PCT/US2008/078593); and/or WO/2010/127336 (PCT/US2010/033345). The assay may, for example, measure alternative or classical pathway-mediated erythrocyte lysis or be an ELISA assay. In some embodiments, an assay described in WO/2010/135717 (PCT/US2010/035871) is used.

Table 1 provides a non-limiting list of compstatin analogs useful in the present disclosure. The analogs are referred to in abbreviated form in the left column by indicating specific modifications at designated positions (1-13) as compared to the parent peptide, compstatin. Consistent with usage in the art, “compstatin” as used herein, and the activities of compstatin analogs described herein relative to that of compstatin, refer to the compstatin peptide amidated at the C-terminus. Unless otherwise indicated, peptides in Table 1 are amidated at the C-terminus. Bold text is used to indicate certain modifications. Activity relative to compstatin is based on published data and assays described therein (WO2004/026328, WO2007044668, Mallik, 2005; Katragadda, 2006). In certain embodiments, the peptides listed in Table 1 are cyclized via a disulfide bond between the two Cys residues when used in the therapeutic compositions and methods of the disclosure. Alternate means for cyclizing the peptides are also within the scope of the disclosure.

TABLE 1

| Peptide | Sequence | SEQ ID NO: | Activity over compstatin |
|---|---|---|---|
| Compstatin | _{H}-ICVVQDWGHHRCT-_{CONH2} | 8 | * |
| Ac-compstatin | _{Ac}-ICVVQDWGHHRCT-_{CONH2} | 9 | 3xmore |
| AC-V4Y/H9A | _{Ac}-ICVYQDWGAHRCT-_{CONH2} | 10 | 14xmore |
| AC-V4W/H9A -OH | _{Ac}-ICVWQDWGAHRCT-_{COOH} | 11 | 27xmore |
| Ac-V4W/H9A | _{Ac}-ICVWQDWGAHRCT-_{CONH2} | 12 | 45xmore |
| AC-V4W/H9A/T13dT -OH | _{Ac}-ICVWQDWGAHRCdT-_{COOH} | 13 | 55xmore |
| Ac-V4(2-Nal)/H9A | _{Ac}-ICV(2-Nal)QDWGAHRCT-_{CONH2} | 14 | 99xmore |
| Ac V4(2-Nal)/H9A -OH | _{Ac}-ICV(2-Nal)QDWGAHRCT-_{COOH} | 15 | 38xmore |
| Ac V4(1-Nal)/H9A -OH | _{Ac}-ICV(1-Nal)QDWGAHRCT-_{COOH} | 16 | 30xmore |
| Ac-V42Igl/H9A | _{Ac}-1CV(2-Igl)QDWGAHRCT-_{CONH2} | 17 | 39xmore |
| Ac-V42Igl/H9A -OH | _{Ac}-ICV(2-Igl)QDWGAHRCT-_{COOH} | 18 | 37xmore |
| Ac-V4Dht/H9A -OH | _{Ac}-ICVDhtQDWGAHRCT-_{COOH} | 19 | 5xmore |
| Ac-V4(Bpa)/H9A -OH | _{Ac}-ICV(Bpa)QDWGAHRCT-_{COOH} | 20 | 49xmore |
| Ac-V4(Bpa)/H9A | _{Ac}-ICV(Bpa)QDWGAHRCT-_{CONH2} | 21 | 86xmore |
| Ac-V4(Bta)/H9A -OH | _{Ac}-ICV(Bta)QDWGAHRCT-_{COOH} | 22 | 65xmore |
| Ac-V4(Bta)/H9A | _{Ac}-ICV(Bta)QDWGAHRCT-_{CONH2} | 23 | 64xmore |
| Ac-V4W/H9(2-Abu) | _{Ac}-ICVWQDWG(2-Abu)HRCT-_{CONH2} | 24 | 64xmore |
| +G/V4W/H9A +AN -OH | _{H}-GICVWQDWGAHRCTAN-_{COOH} | 25 | 38xmore |
| Ac-V4(5fW)/H9A | _{Ac}-ICV(5fW)QDWGAHRCT- _{CONH2} | 26 | 31xmore |
| Ac-V4(5-MeW)/H9A | _{Ac}-ICV(5-methyl-W)QDWGAHRCT- _{CONH2} | 27 | 67xmore |
| Ac-V4(1-MeW)/H9A | _{Ac}-ICV(1-methyl-W)QDWGAHRCT- _{CONH2} | 28 | 264xmore |
| Ac-V4W/W7(5fW)/H9A | _{Ac}-ICVWQD(5fW)GAHRCT-_{CONH2} | 29 | 121xmore |
| Ac-V4(5fW)/W7(5fW)/H9A | _{Ac}-ICV(5fW)QD(5fW)GAHRCT- _{CONH2} | 30 | NA |
| Ac-V4(5-MeW)/W7(5fW)H9A | _{Ac}-ICV(5-methyl-W)QD(5fW)GAHRCT- _{CONH2} | 31 | NA |
| Ac-V4(1MeW)/W7(5fW)/H9A | _{Ac}-ICV(1-methyl-W)QD(5fW)GAHRCT-_{CONH2} | 32 | 264xmore |
| +G/V4(6fW)/W7(6fW)H9A+N -OH | _{H}-GICV(6fW)QD(6fW)GAHRCTN-_{COOH} | 33 | 126xmore |
| Ac-V4(1-formyl-W)/H9A | _{Ac}-ICV(1-formyl-W)QDWGAHRCT-_{CONH2} | 34 | 264xmore |
| Ac-V4(5-methoxy-W)/H9A | _{Ac}-ICV(1-methyoxy-W)QDWGAHRCT- _{CONH2} | 35 | 76xmore |
| G/V4(5f-W)/W7(5fW)/H9A+N - OH | _{H}-GICV(5fW)QD(5fW)GAHRCTN-_{COOH} | 36 | 112xmore |

NA = not available

In certain embodiments of the compositions and methods of the disclosure, the compstatin analog has a sequence selected from sequences 9-36. In some embodiments, the compstatin analog has a sequence of SEQ ID NO: 28. As used herein, “L-amino acid” refers to any of the naturally occurring levorotatory alpha-amino acids normally present in proteins or the alkyl esters of those alpha-amino acids. The term “D-amino acid” refers to dextrorotatory alpha-amino acids. Unless specified otherwise, all amino acids referred to herein are L-amino acids.

In some embodiments, one or more amino acid(s) of a compstatin analog (e.g., any of the compstatin analogs disclosed herein) can be an N-alkyl amino acid (e.g., an N-methyl amino acid). For example, and without limitation, at least one amino acid within the cyclic portion of the peptide, at least one amino acid N-terminal to the cyclic portion, and/or at least one amino acid C-terminal to the cyclic portion may be an N-alkyl amino acid, e.g., an N-methyl amino acid. In some embodiments, for example, a compstatin analog comprises an N-methyl glycine, e.g., at the position corresponding to position 8 of compstatin and/or at the position corresponding to position 13 of compstatin. In some embodiments, one or more of the compstatin analogs in Table 1 contains at least one N-methyl glycine, e.g., at the position corresponding to position 8 of compstatin and/or at the position corresponding to position 13 of compstatin. In some embodiments, one or more of the compstatin analogs in Table 1 contains at least one N-methyl isoleucine, e.g., at the position corresponding to position 13 of compstatin. For example, a Thr at or near the C-terminal end of a peptide whose sequence is listed in Table 1 or any other compstatin analog sequence may be replaced by N-methyl Ile. As will be appreciated, in some embodiments the N-methylated amino acids comprise N-methyl Gly at position 8 and N-methyl Ile at position 13.

Compstatin analogs may be prepared by various synthetic methods of peptide synthesis known in the art via condensation of amino acid residues, e.g., in accordance with conventional peptide synthesis methods, may be prepared by expression in vitro or in living cells from appropriate nucleic acid sequences encoding them using methods known in the art. For example, peptides may be synthesized using standard solid-phase methodologies as described in Malik, supra, Katragadda, supra, WO2004026328, and/or WO2007062249. Potentially reactive moieties such as amino and carboxyl groups, reactive functional groups, etc., may be protected and subsequently deprotected using various protecting groups and methodologies known in the art. See, e.g., “Protective Groups in Organic Synthesis”, 3^{rd} ed. Greene, T. W. and Wuts, P. G., Eds., John Wiley & Sons, New York: 1999. Peptides may be purified using standard approaches such as reversed-phase HPLC. Separation of diasteriomeric peptides, if desired, may be performed using known methods such as reversed-phase HPLC. Preparations may be lyophilized, if desired, and subsequently dissolved in a suitable solvent, e.g., water. The pH of the resulting solution may be adjusted, e.g., to physiological pH, using a base such as NaOH. Peptide preparations may be characterized by mass spectrometry if desired, e.g., to confirm mass and/or disulfide bond formation. See, e.g., Mallik, 2005, and Katragadda, 2006.

A compstatin analog can be modified by addition of a molecule such as polyethylene glycol (PEG) to stabilize the compound, reduce its immunogenicity, increase its lifetime in the body, increase or decrease its solubility, and/or increase its resistance to degradation. Methods for pegylation are well known in the art (Veronese, F.M. & Harris, Adv. Drug Deliv. Rev. 54, 453-456, 2002; Davis, F.F., Adv. Drug Deliv. Rev. 54, 457-458, 2002); Hinds, K.D. & Kim, S.W. Adv. Drug Deliv. Rev. 54, 505-530 (2002; Roberts, M.J., Bentley, M.D. & Harris, J.M. Adv. Drug Deliv. Rev. 54, 459-476; 2002); Wang, Y.S. et al. Adv. Drug Deliv. Rev. 54, 547-570, 2002). A wide variety of polymers such as PEGs and modified PEGs, including derivatized PEGs to which polypeptides can conveniently be attached are described in Nektar Advanced Pegylation 2005-2006 Product Catalog, Nektar Therapeutics, San Carlos, CA, which also provides details of appropriate conjugation procedures.

In some embodiments, a compstatin analog of any of SEQ ID NOs: 9-36, is extended by one or more amino acids at the N-terminus, C-terminus, or both, wherein at least one of the amino acids has a side chain that comprises a reactive functional group such as a primary or secondary amine, a sulfhydryl group, a carboxyl group (which may be present as a carboxylate group), a guanidino group, a phenol group, an indole ring, a thioether, or an imidazole ring, which facilitate conjugation with a reactive functional group to attach a PEG to the compstatin analog. In some embodiments, the compstatin analog comprises an amino acid having a side chain comprising a primary or secondary amine, e.g., a Lys residue. For example, a Lys residue, or a sequence comprising a Lys residue, is added at the N-terminus and/or C-terminus of a compstatin analog described herein (e.g., a compstatin analog comprising any one of SEQ ID NOs: 9-36).

In some embodiments, the Lys residue is separated from the cyclic portion of the compstatin analog by a rigid or flexible spacer. The spacer may, for example, comprise a substituted or unsubstituted, saturated or unsaturated alkyl chain, oligo(ethylene glycol) chain, and/or other moieties, e.g., as described herein with regard to linkers. The length of the chain may be, e.g., between 2 and 20 carbon atoms. In other embodiments the spacer is a peptide. The peptide spacer may be, e.g., between 1 and 20 amino acids in length, e.g., between 4 and 20 amino acids in length. Suitable spacers can comprise or consist of multiple Gly residues, Ser residues, or both, for example. Optionally, the amino acid having a side chain comprising a primary or secondary amine and/or at least one amino acid in a spacer is a D-amino acid. Any of a variety of polymeric backbones or scaffolds could be used. For example, the polymeric backbone or scaffold may be a polyamide, polysaccharide, polyanhydride, polyacrylamide, polymethacrylate, polypeptide, polyethylene oxide, or dendrimer. Suitable methods and polymeric backbones are described, e.g., in WO98/46270 (PCT/US98/07171) or WO98/47002 (PCT/US98/06963). In some embodiments, the polymeric backbone or scaffold comprises multiple reactive functional groups, such as carboxylic acids, anhydride, or succinimide groups. The polymeric backbone or scaffold is reacted with the compstatin analogs. In some embodiments, the compstatin analog comprises any of a number of different reactive functional groups, such as carboxylic acids, anhydride, or succinimide groups, which are reacted with appropriate groups on the polymeric backbone. Alternately, monomeric units that could be joined to one another to form a polymeric backbone or scaffold are first reacted with the compstatin analogs and the resulting monomers are polymerized. In some embodiments, short chains are prepolymerized, functionalized, and then a mixture of short chains of different composition are assembled into longer polymers.

In some embodiments, a compstatin analog moiety is attached at each end of a linear PEG. A bifunctional PEG having a reactive functional group at each end of the chain may be used, e.g., as described herein. In some embodiments, the reactive functional groups are identical while in some embodiments different reactive functional groups are present at each end.

In general and for compounds depicted herein, a polyethylene glycol moiety is drawn with the oxygen atom on the right side of the repeating unit or the left side of the repeating unit. In cases where only one orientation is drawn, the present disclosure encompasses both orientations (i.e., (CH₂CH₂O)ₙ and (OCH₂CH₂)ₙ) of polyethylene glycol moieties for a given compound or genus, or in cases where a compound or genus contains multiple polyethylene glycol moieties, all combinations of orientations are encompasses by the present disclosure.

In some embodiments a bifunctional linear PEG comprises a moiety comprising a reactive functional group at each of its ends. The reactive functional groups may be the same (homobifunctional) or different (heterobifunctional). In some embodiments the structure of a bifunctional PEG may be symmetric, wherein the same moiety is used to connect the reactive functional group to oxygen atoms at each end of the -(CH₂CH₂O)ₙ chain. In some embodiments different moieties are used to connect the two reactive functional groups to the PEG portion of the molecule. The structures of exemplary bifunctional PEGs are depicted below. For illustrative purposes, formulas in which the reactive functional group(s) comprise an NHS ester are depicted, but other reactive functional groups could be used.

In some embodiments, a bifunctional linear PEG is of formula A:

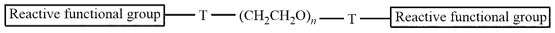

wherein each T and “Reactive functional group” is independently as defined below, and described in classes and subclasses herein, and n is as defined above and described in classes and subclasses herein.

Each T is independently a covalent bond or a C₁₋₁₂ straight or branched, hydrocarbon chain wherein one or more carbon units of T are optionally and independently replaced by —O—, —S—, —N(R^{x})—, —C(O)—, —C(O)O—, —OC(O)—, —N(R^{x})C(O)—, —C(O)N(R^{x})—, —S(O)—, —S(O)₂—, —N(R^{x})SO₂—, or —SO₂N(R^{x})—; and each R^{x} is independently hydrogen or C₁₋₆ aliphatic.

The Reactive functional group has the structure —COO—NHS.

Exemplary bifunctional PEGs of formula A include:

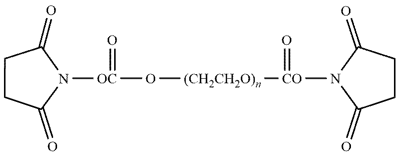

In some embodiments, a functional group (for example, an amine, hydroxyl, or thiol group) on a compstatin analog is reacted with a PEG-containing compound having a “reactive functional group” as described herein, to generate such conjugates. By way of example, Formula I can form compstatin analog conjugates having the structure:

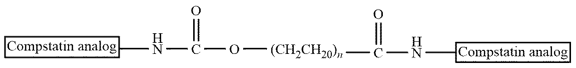

wherein,

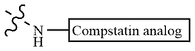

represents the attachment point of an amine group on a compstatin analog. In certain embodiments, an amine group is a lysine side chain group.

In certain embodiments, the PEG component of such conjugates has an average molecular weight of about 5 kD, about 10 kD, about 15 kD, about 20 kD, about 30 kD, or about 40 kD. In certain embodiments, the PEG component of such conjugates has an average molecular weight of about 40 kD.

The term “bifunctional” or “bifunctionalized” is sometimes used herein to refer to a compound comprising two compstatin analog moieties linked to a PEG. Such compounds may be designated with the letter “BF”. In some embodiments a bifunctionalized compound is symmetrical. In some embodiments the linkages between the PEG and each of the compstatin analog moieties of a bifunctionalized compound are the same. In some embodiments, each linkage between a PEG and a compstatin analog of a bifunctionalized compound comprises a carbamate. In some embodiments, each linkage between a PEG and a compstatin analog of a bifunctionalized compound comprises a carbamate and does not comprise an ester. In some embodiments, each compstatin analog of a bifunctionalized compound is directly linked to a PEG via a carbamate. In some embodiments, each compstatin analog of a bifunctionalized compound is directly linked to a PEG via a carbamate, and the bifunctionalized compound has the structure:

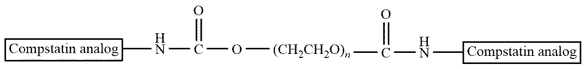

In some embodiments of formulae and embodiments described herein,

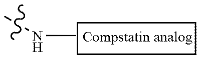

represents point of attachment of a lysine side chain group in a compstatin analog having the structure:

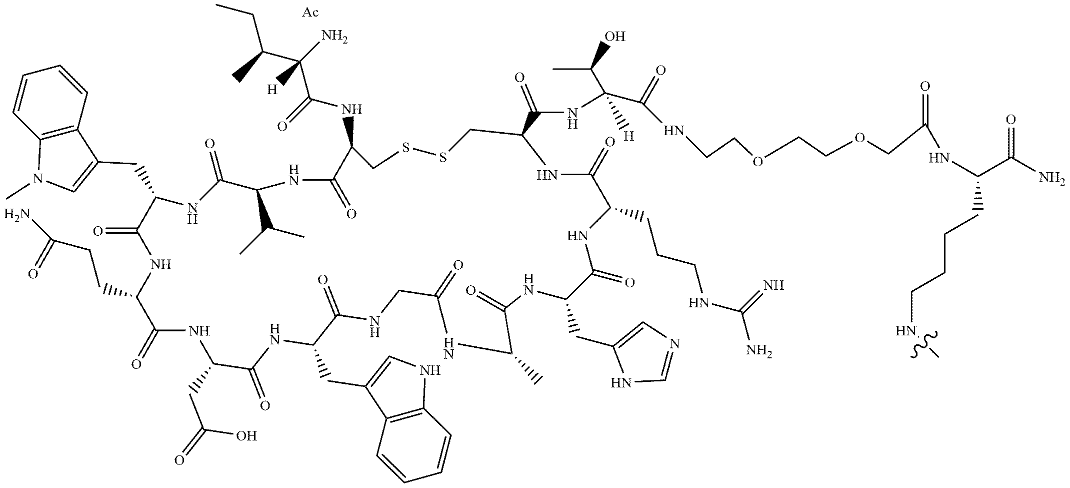

wherein the symbol “〰” denotes the point of attachment of a chemical moiety to the remainder of a molecule or chemical formula.

PEGs comprising one or more reactive functional groups may, in some embodiments, be obtained from, e.g., NOF America Corp. White Plains, NY or BOC Sciences 45-16 Ramsey Road Shirley, NY 11967, USA, among others, or may be prepared using methods known in the art.

In some embodiments, a linker is used to connect a compstatin analog described herein and a PEG described herein. Suitable linkers for connecting a compstatin analog and a PEG are extensively described above and in classes and subclasses herein. In some embodiments, a linker has multiple functional groups, wherein one functional group is connected to a compstatin analog and another is connected to a PEG moiety. In some embodiments, a linker is a bifunctional compound. In some embodiments, a linker has the structure of NH₂(CH₂CH₂O)nCH₂C(═O)OH, wherein n is 1 to 1000. In some embodiments, a linker is 8-amino-3,6-dioxaoctanoic acid (AEEAc). In some embodiments, a linker is activated for conjugation with a polymer moiety or a functional group of a compstatin analog. For example, in some embodiments, the carboxyl group of AEEAc is activated before conjugation with the amine group of the side chain of a lysine group.

In some embodiments, a suitable functional group (for example, an amine, hydroxyl, thiol, or carboxylic acid group) on a compstatin analog is used for conjugation with a PEG moiety, either directly or via a linker. In some embodiments, a compstatin analog is conjugated through an amine group to a PEG moiety via a linker. In some embodiments, an amine group is the α-amino group of an amino acid residue. In some embodiments, an amine group is the amine group of the lysine side chain. In some embodiments, a compstatin analog is conjugated to a PEG moiety through the amino group of a lysine side chain (ε-amino group) via a linker having the structure of NH₂(CH₂CH₂O)nCH₂C(═O)OH, wherein n is 1 to 1000. In some embodiments, a compstatin analog is conjugated to the PEG moiety through the amino group of a lysine side chain via an AEEAc linker. In some embodiments, the NH₂(CH₂CH₂O)nCH₂C(═O)OH linker introduces a —NH(CH₂CH₂O)nCH₂C(═O)— moiety on a compstatin lysine side chain after conjugation. In some embodiments, the AEEAc linker introduces a —NH(CH₂CH₂O)₂CH₂C(═O)— moiety on a compstatin lysine side chain after conjugation.

In some embodiments, a compstatin analog is conjugated to a PEG moiety via a linker, wherein the linker comprises an AEEAc moiety and an amino acid residue. In some embodiments, a compstatin analog is conjugated to a PEG moiety via a linker, wherein the linker comprises an AEEAc moiety and a lysine residue. In some embodiments, the C-terminus of a compstatin analog is connected to the amino group of AEEAc, and the C-terminus of AEEAc is connected to a lysine residue. In some embodiments, the C-terminus of a compstatin analog is connected to the amino group of AEEAc, and the C-terminus of AEEAc is connected to the α-amino group of a lysine residue. In some embodiments, the C-terminus of a compstatin analog is connected to the amino group of AEEAc, the C-terminus of AEEAc is connected to the α-amino group of the lysine residue, and a PEG moiety is conjugated through the ε-amino group of said lysine residue. In some embodiments, the C-terminus of the lysine residue is modified. In some embodiments, the C-terminus of the lysine residue is modified by amidation. In some embodiments, the N-terminus of a compstatin analog is modified. In some embodiments, the N-terminus of a compstatin analog is acetylated.

In certain embodiments a compstatin analog may be represented as M-AEEAc-Lys-B₂, wherein B₂ is a blocking moiety, e.g., NH₂, M represents any of SEQ ID NOs: 9-36, , with the proviso that the C-terminal amino acid of any of SEQ ID NOs: 9-36 is linked via a peptide bond to AEEAc-Lys-B₂. The NHS moiety of a monofunctional or multifunctional (e.g., bifunctional) PEG reacts with the free amine of the lysine side chain to generate a monofunctionalized (one compstatin analog moiety) or multifunctionalized (multiple compstatin analog moieties) PEGylated compstatin analog. In various embodiments any amino acid comprising a side chain that comprises a reactive functional group may be used instead of Lys (or in addition to Lys). A monofunctional or multifunctional PEG comprising a suitable reactive functional group may be reacted with such side chain in a manner analogous to the reaction of NHS-ester activated PEGs with Lys.

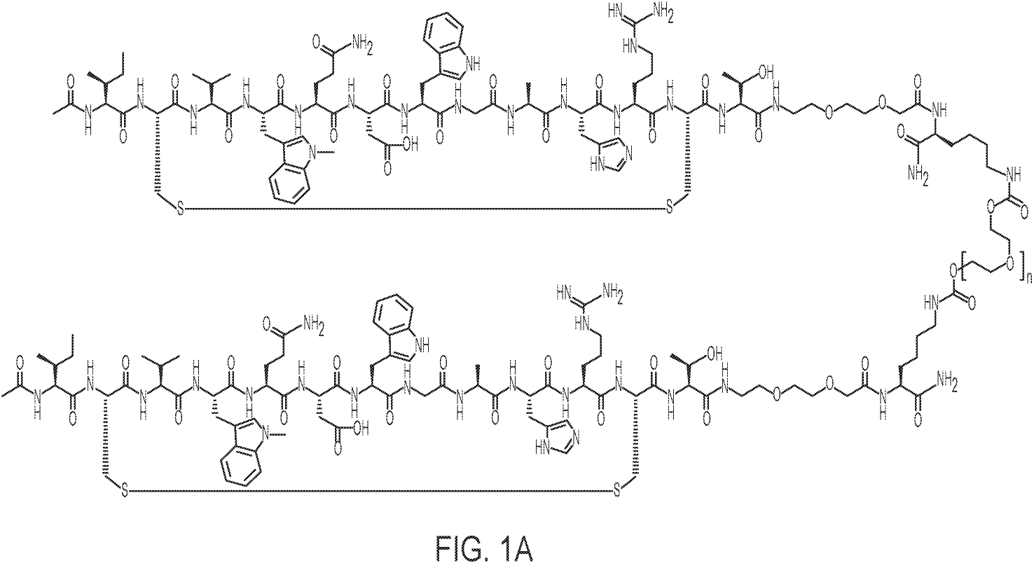

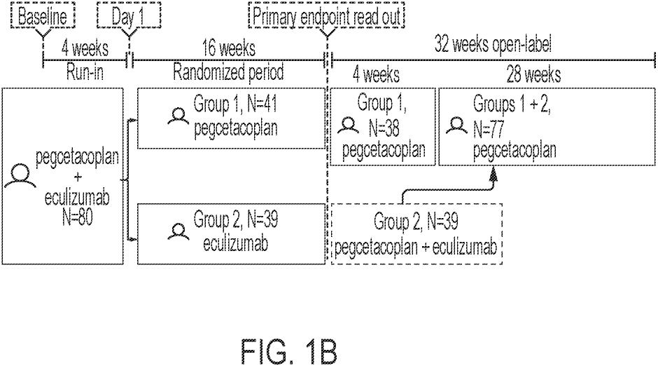

With regard to any of the above formulae and structures, it is to be understood that embodiments in which the compstatin analog component comprises any compstatin analog described herein, e.g., any compstatin analog of SEQ ID NOs; 9-36 are expressly disclosed. For example, and without limitation, a compstatin analog may comprise the amino acid sequence of SEQ ID NO: 28. An exemplary PEGylated compstatin analog in which the compstatin analog component comprises the amino acid sequence of SEQ ID NO: 28 is depicted in FIG. 1A. It will be understood that the PEG moiety may have a variety of different molecular weights or average molecular weights in various embodiments, as described herein. In certain embodiments, a compstatin analog is pegcetacoplan (“APL-2”), having the structure of the compound of FIG. 1A with n of about 800 to about 1100 and a PEG having an average molecular weight of about 40 kD. Pegcetacoplan is also referred to as Poly(oxy-1,2-ethanediyl), α-hydro-ω-hydroxy-, 15,15ʹ -diester with N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy)ethoxy]acetyl-N⁶-carboxy-L-lysinamide cyclic (2-->12)-(disulfide); or O,Oʹ-bis[(S²,S¹²-cyclo{N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy)ethoxy]acetyl-L-lysinamide})-N^{6.15}- carbonyl]polyethylene glycol (n = 800-1100). Additional compstatin analogs are described in, e.g., WO 2012/155107 and WO 2014/078731.

### III. Treatment Methods

In some embodiments, a compstatin analog described herein, e.g., pegcetacoplan, is used to treat paroxysmal nocturnal hemoglobinuria (PNH). In some embodiments, a compstatin analog described herein, e.g., pegcetacoplan, is administered to a subject having or suffering from PNH. In some embodiments, a compstatin analog described herein, e.g., pegcetacoplan, is administered, e.g., subcutaneously, to the subject at about 800 mg to about 1200 mg, e.g., about 1060 mg to about 1100 mg, e.g., about 1070 mg to about 1090 mg, e.g., about 1075 mg to about 1085 mg, e.g., about 1080 mg. In some embodiments, a compstatin analog described herein, e.g., pegcetacoplan, is administered, e.g., subcutaneously, to the subject twice weekly at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg. In some embodiments, a compstatin analog described herein, e.g., pegcetacoplan, is administered, e.g., subcutaneously, to the subject every three days at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg. In some embodiments, a compstatin analog described herein, e.g., pegcetacoplan, is administered, e.g., subcutaneously, to the subject, e.g., twice weekly, at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg, for about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years, 3 years, 4 years, 5 years, or longer. In some embodiments, a compstatin analog described herein, e.g., pegcetacoplan, is administered, e.g., subcutaneously, to the subject, e.g., every 3 days, at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg, for about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years, 3 years, 4 years, 5 years, or longer. In some embodiments, a compstatin analog described herein, e.g., pegcetacoplan, is administered, e.g., subcutaneously, to a pediatric subject (e.g., 12-17 years of age, with a body weight of about 20-34 kg) at a dosage of about 300 mg to about 750 mg (e.g., about 400 mg to about 650 mg, e.g., about 500 mg to about 600 mg, e.g., about 540 mg) twice weekly for two initial doses followed by a dosage of about 400 mg to about 850 mg (e.g., about 500 mg to about 750 mg, e.g., about 600 mg to about 700 mg, e.g., about 648 mg) twice weekly. In some embodiments, a compstatin analog described herein, e.g., pegcetacoplan, is administered, e.g., subcutaneously, to a pediatric subject (e.g., 12-17 years of age, with a body weight of about 35-49 kg) at a single dose of about 400 mg to about 850 mg (e.g., about 500 mg to about 750 mg, e.g., about 600 mg to about 700 mg, e.g., about 648 mg) followed by a dosage of about 500 mg to about 1000 mg (e.g., about 600 mg to about 900 mg, e.g., about 700 mg to about 850 mg, e.g., about 810 mg) twice weekly. In some embodiments, a compstatin analog described herein, e.g., pegcetacoplan, is administered, e.g., subcutaneously, to a pediatric subject (e.g., 12-17 years of age, with a body weight of about 20-34 kg) at a dosage of about 400 mg to about 850 mg (e.g., about 500 mg to about 750 mg, e.g., about 600 mg to about 700 mg, e.g., about 648 mg) every 3 days. In some embodiments, a compstatin analog described herein, e.g., pegcetacoplan, is administered, e.g., subcutaneously, to a pediatric subject (e.g., 12-17 years of age, with a body weight of about 35-49 kg) at a dosage of about 500 mg to about 1000 mg (e.g., about 600 mg to about 900 mg, e.g., about 700 mg to about 850 mg, e.g., about 810 mg) every 3 days. In some embodiments, a specific improvement (e.g., a statistically significant or clinically significant improvement) of one or more PNH symptoms or parameters is achieved in the subject, e.g., one or more target levels described herein is achieved.

In some embodiments, a compstatin analog described herein, e.g., pegcetacoplan, is administered to a population of subjects having or suffering from PNH. In some embodiments, a compstatin analog described herein, e.g., pegcetacoplan, is administered, e.g., subcutaneously, to the population of subjects at about 800 mg to about 1200 mg, e.g., about 1060 mg to about 1100 mg, e.g., about 1070 mg to about 1090 mg, e.g., about 1075 mg to about 1085 mg, e.g., about 1080 mg. In some embodiments, a compstatin analog described herein, e.g., pegcetacoplan, is administered, e.g., subcutaneously, to the population of subjects twice weekly at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg. In some embodiments, a compstatin analog described herein, e.g., pegcetacoplan, is administered, e.g., subcutaneously, to the population of subjects every three days at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg. In some embodiments, a compstatin analog described herein, e.g., pegcetacoplan, is administered, e.g., subcutaneously, to the population of subjects, e.g., twice weekly, at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg, for about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years, 3 years, 4 years, 5 years, or longer. In some embodiments, a compstatin analog described herein, e.g., pegcetacoplan, is administered, e.g., subcutaneously, to the population of subjects, e.g., every 3 days, at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg, for about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years, 3 years, 4 years, 5 years, or longer. In some embodiments, a compstatin analog described herein, e.g., pegcetacoplan, is administered, e.g., subcutaneously, to a population of pediatric subjects (e.g., 12-17 years of age, with a body weight of about 20-34 kg) at a dosage of about 300 mg to about 750 mg (e.g., about 400 mg to about 650 mg, e.g., about 500 mg to about 600 mg, e.g., about 540 mg) twice weekly for two initial doses followed by a dosage of about 400 mg to about 850 mg (e.g., about 500 mg to about 750 mg, e.g., about 600 mg to about 700 mg, e.g., about 648 mg) twice weekly. In some embodiments, a compstatin analog described herein, e.g., pegcetacoplan, is administered, e.g., subcutaneously, to a population of pediatric subjects (e.g., 12-17 years of age, with a body weight of about 35-49 kg) at a single dose of about 400 mg to about 850 mg (e.g., about 500 mg to about 750 mg, e.g., about 600 mg to about 700 mg, e.g., about 648 mg) followed by a dosage of about 500 mg to about 1000 mg (e.g., about 600 mg to about 900 mg, e.g., about 700 mg to about 850 mg, e.g., about 810 mg) twice weekly. In some embodiments, a compstatin analog described herein, e.g., pegcetacoplan, is administered, e.g., subcutaneously, to a population of pediatric subjects (e.g., 12-17 years of age, with a body weight of about 20-34 kg) at a dosage of about 400 mg to about 850 mg (e.g., about 500 mg to about 750 mg, e.g., about 600 mg to about 700 mg, e.g., about 648 mg) every 3 days. In some embodiments, a compstatin analog described herein, e.g., pegcetacoplan, is administered, e.g., subcutaneously, to a population of pediatric subjects (e.g., 12-17 years of age, with a body weight of about 35-49 kg) at a dosage of about 500 mg to about 1000 mg (e.g., about 600 mg to about 900 mg, e.g., about 700 mg to about 850 mg, e.g., about 810 mg) every 3 days. In some embodiments, an average level of a specific improvement (e.g., a statistically significant or clinically significant improvement) of one or more PNH symptoms or parameters is achieved in the population of subjects, e.g., the population of subjects, on average, achieves one or more target levels described herein.

For example, in some embodiments, administration of a compstatin analog described herein, e.g., pegcetacoplan, to a subject (or to a population of subjects) increases hemoglobin level in the subject (or increases average hemoglobin levels in the population of subjects) to a target hemoglobin level. In some embodiments, the target hemoglobin level is a hemoglobin level that is higher, relative to a control hemoglobin level, by at least about 1 g/dL, e.g., by at least about 2 g/dL, e.g., by at least 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 g/dL. In some embodiments, the target hemoglobin level is a hemoglobin level that is higher, relative to a control hemoglobin level, by about 1 g/dL to about 4 g/dL, e.g., by about 2 g/dL to about 3 g/dL, e.g., about 2.4 g/dL. In some embodiments, the target hemoglobin level is a hemoglobin level that is higher, relative to a control hemoglobin level, by at least about 20%, 40%, 60%, 80%, 100%, or more. In some embodiments, the control hemoglobin level is a hemoglobin level in a subject suffering from PNH (or an average hemoglobin level in a population of subjects suffering from PNH) and not receiving the compstatin analog (e.g., a subject or population of subjects receiving a C5 inhibitor, e.g., eculizumab); a hemoglobin level in the subject (or an average hemoglobin level in the population of subjects) before receiving the compstatin analog; or a lower limit of a range of hemoglobin levels in a healthy subject (e.g., about 12 g/dL). In some embodiments, a range of hemoglobin levels in a healthy subject is a gender-specific range. In some embodiments, the target hemoglobin level is about 10 g/dL to about 15 g/dL, e.g., about 11 g/dL, about 12 g/dL, or about 13 g/dL. In some embodiments, the target hemoglobin level is a hemoglobin level that is at least the lower limit of the normal range of Hb level, e.g., at least the lower limit of the gender-specific normal range for that subject. In some embodiments, the target hemoglobin level is achieved after about 1, 2, 3, 4, 5, 6, 7, 8, 9, 10, or more doses. In some embodiments, the target hemoglobin level is achieved after about 1 day, 1 week, 2 weeks, 3 weeks, 4 weeks, 6 weeks, 8 weeks, or more, of treatment. For example, in some embodiments, the target Hb level is reached after about 2, about 3, or about 4 weeks of treatment with the complement inhibitor described herein, e.g., pegcetacoplan. In some embodiments, the target hemoglobin level is sustained for about 1 day, 1 week, 2 weeks, 3 weeks, 4 weeks, 6 weeks, 8 weeks, or more, following at least 1 dose of the compstatin analog described herein, e.g., pegcetacoplan. In some embodiments, for example, the target Hb level is sustained for a time period of at least 16 weeks following initiation of treatment with the compstatin analog described herein, e.g., pegcetacoplan, using a dosing regimen described herein, e.g., about 1080 mg administered subcutaneously twice weekly or every three days, wherein the subject (or population of subjects) remains under treatment with the compstatin analog during said time period and is not treated with a C5 inhibitor during said time period. Hemoglobin levels can be assessed using standard methods known in the art.

In some embodiments, administration of a compstatin analog described herein, e.g., pegcetacoplan, to a subject (or to a population of subjects) reduces number of transfusions needed by the subject (or reduces average number of transfusions needed by the population of subjects) to a target number of transfusions. In some embodiments, the target number of transfusions is at least 1 (e.g., at least 2, 3, 4, 5, 6 or more) fewer transfusions over a defined period of time relative to a control number of transfusions. In some embodiments, the control number of transfusions is a number of transfusions administered to a subject suffering from PNH (or an average number of transfusions administered to a population of subjects suffering from PNH) and not receiving a compstatin analog described herein, e.g., pegcetacoplan (e.g., a subject or population of subjects receiving a C5 inhibitor, e.g., eculizumab); or a number of transfusions administered to the subject (or average number of transfusions administered to the population of subjects) before receiving a compstatin analog described herein, e.g., pegcetacoplan. In some embodiments, the target number of transfusions is fewer than 3, 2, or 1 transfusions over about 4 weeks, 8 weeks, 12 weeks, 16 weeks, 20 weeks, 24 weeks, 32 weeks, 52 weeks, or more. In some embodiments, the target number of transfusions is achieved after about 1 week, 2 weeks, 3 weeks, 4 weeks, 6 weeks, 8 weeks, or more, of treatment. In some embodiments, the target number of transfusions is sustained for about 1 week, 2 weeks, 3 weeks, 4 weeks, 6 weeks, 8 weeks, or more, following at least 1 dose of the compstatin analog described herein, e.g., pegcetacoplan. In some embodiments, for example, the target number of transfusions is sustained for a time period of at least 16 weeks after initiation of treatment with the compstatin analog described herein, e.g., pegcetacoplan, using a dosing regimen described herein, e.g., about 1080 mg administered subcutaneously twice weekly or every three days, wherein the subject (or population of subjects) remains under treatment with the compstatin analog during said time period and is not treated with a C5 inhibitor during said time period.

In some embodiments, administration of a compstatin analog described herein, e.g., pegcetacoplan, to a subject (or to a population of subjects) reduces number of PRBC units needed by the subject (or reduces average number of PRBC units needed by the population of subjects), e.g., to a target number of PRBC units. In some embodiments, the target number of PRBC units is at least 1 (e.g., at least 2, 3, 4, 5, 6 or more) fewer PRBC units over a defined period of time relative to a control number of PRBC units. In some embodiments, the control number of PRBC units is a number of PRBC units administered to a subject suffering from PNH (or an average number of PRBC units administered to a population of subjects suffering from PNH) and not receiving a compstatin analog described herein, e.g., pegcetacoplan (e.g., a subject or population of subjects receiving a C5 inhibitor, e.g., eculizumab); or a number of PRBC units administered to the subject (or an average number of PRBC units administered to the population of subjects) before receiving a compstatin analog described herein, e.g., pegcetacoplan. In some embodiments, the target number of PRBC units is fewer than 3, 2, or 1 PRBC units over about 4 weeks, 8 weeks, 12 weeks, 16 weeks, 20 weeks, 24 weeks, or more. In some embodiments, the number of PRBC units is achieved after about 1 day, 1 week, 2 weeks, 3 weeks, 4 weeks, 6 weeks, 8 weeks, or more, of treatment. In some embodiments, the target number of PRBC units is sustained for about 1 day, 1 week, 2 weeks, 3 weeks, 4 weeks, 6 weeks, 8 weeks, 16 weeks, or more, following at least 1 dose of the compstatin analog described herein, e.g., pegcetacoplan.

In some embodiments, administration of a compstatin analog described herein, e.g., pegcetacoplan, to a subject (or to a population of subjects) reduces number of reticulocytes in the blood of a subject (or reduces average number of reticulocytes in the blood of the population of subjects) to a target reticulocyte level. In some embodiments, the target reticulocyte level is a reticulocyte level that is lower, relative to a control reticulocyte level, by at least about 20%, 40%, 60%, or 80%. In some embodiments, the control reticulocyte level is a reticulocyte level in a subject suffering from PNH (or an average reticulocyte level in a population of subjects suffering from PNH) and not receiving a compstatin analog described herein, e.g., pegcetacoplan (e.g., a subject or population of subjects receiving a C5 inhibitor, e.g., eculizumab); a reticulocyte level in the subject (or an average reticulocyte level in the population of subjects) before receiving a compstatin analog described herein, e.g., pegcetacoplan; or an upper limit of a range of reticulocyte levels in a healthy subject (e.g., a range of reticulocyte levels in a healthy subject of 30-120 × 10⁹ /L). In some embodiments, a range of reticulocyte levels in a healthy subject is a gender-specific range. In some embodiments, the target reticulocyte level is about 30 to about 100 × 10⁹ /L, e.g., about 70, 80, or 90 × 10⁹ /L. In some embodiments, the target reticulocyte level is achieved after about 1 day, 1 week, 2 weeks, 3 weeks, 4 weeks, 6 weeks, 8 weeks, or more, of treatment. In some embodiments, the target reticulocyte level is sustained for about 1 day, 1 week, 2 weeks, 3 weeks, 4 weeks, 6 weeks, 8 weeks, 16 weeks, or more, following at least 1 dose of the compstatin analog described herein, e.g., pegcetacoplan.

In some embodiments, administration of a compstatin analog described herein, e.g., pegcetacoplan, to a subject (or to a population of subjects) reduces lactate dehydrogenase (LDH) level in the subject (or reduces average LDH level in the population of subjects), e.g., to a target LDH level. In some embodiments, the target LDH level is an LDH level that is lower, relative to a control LDH level, by at least about 20%, 40%, 60%, or 80%. In some embodiments, the target LDH level is an LDH level that is lower, relative to a control LDH level, by at least about 10, 20, 30, 40, 50, 60, 70, 80, 90, or 100 U/L. In some embodiments, the control LDH level is an LDH level in a subject suffering from PNH (or an average LDH level in a population of subjects suffering from PNH) and not receiving a compstatin analog described herein, e.g., pegcetacoplan (e.g., a subject or population of subjects receiving a C5 inhibitor, e.g., eculizumab); an LDH level in the subject (or an average LDH level in the population of subjects) before receiving a compstatin analog described herein, e.g., pegcetacoplan; or an upper limit of a range of LDH levels in a healthy subject (e.g., a range of LDH in a healthy subject of about 113-226 U/L). In some embodiments, a range of LDH levels in a healthy subject is a gender-specific range. In some embodiments, the target LDH level is about 110 to about 225 U/L, e.g., about 120, 140, 160, 180, 200, or 220 U/L. In some embodiments, the target LDH level is achieved after about 1 day, 1 week, 2 weeks, 3 weeks, 4 weeks, 6 weeks, 8 weeks, 16 weeks, or more, of treatment. In some embodiments, the target LDH level is sustained for about 1 day, 1 week, 2 weeks, 3 weeks, 4 weeks, 6 weeks, 8 weeks, or more, following at least 1 dose of the compstatin analog described herein, e.g., pegcetacoplan.

In some embodiments, administration of a compstatin analog described herein, e.g., pegcetacoplan, to a subject (or to a population of subjects) receiving a C5 inhibitor, e.g., eculizumab, maintains LDH level in the subject (or maintains average LDH level in the population of subjects), e.g., at a target LDH level, e.g., LDH level in the subject changes by no more than 5%, 10%, or 15%, relative to level before treatment with the compstatin analog.

In some embodiments, administration of a compstatin analog described herein, e.g., pegcetacoplan, to a subject (or to a population of subjects) reduces indirect (unconjugated) bilirubin level in the subject (or reduces average indirect bilirubin level in the population of subjects), e.g., to a target indirect bilirubin level. In some embodiments, the target indirect bilirubin level is an indirect bilirubin level that is lower, relative to a control indirect bilirubin level, by at least about 20%, 40%, 60%, or 80%. In some embodiments, the control indirect bilirubin level is an indirect bilirubin level in a subject suffering from PNH (or an average indirect bilirubin level in a population of subjects suffering from PNH) and not receiving a compstatin analog described herein, e.g., pegcetacoplan (e.g., a subject or population of subjects receiving a C5 inhibitor, e.g., eculizumab); an indirect bilirubin level in the subject (or an average indirect bilirubin level in the population of subjects) before receiving a compstatin analog described herein, e.g., pegcetacoplan; or an upper limit of a range of indirect bilirubin levels in a healthy subject. In some embodiments, a range of indirect bilirubin levels in a healthy subject is a gender-specific range. In some embodiments, the target indirect bilirubin level is achieved after about 1 day, 1 week, 2 weeks, 3 weeks, 4 weeks, 6 weeks, 8 weeks, or more, of treatment. In some embodiments, the target indirect bilirubin level is sustained for about 1 day, 1 week, 2 weeks, 3 weeks, 4 weeks, 6 weeks, 8 weeks, 16 weeks, or more, following at least 1 dose of the compstatin analog described herein, e.g., pegcetacoplan.

In some embodiments, administration of a compstatin analog described herein, e.g., pegcetacoplan, to a subject (or to a population of subjects) increases haptoglobin level in the subject (or increases average haptoglobin levels in the population of subjects) to a target haptoglobin level. In some embodiments, the target haptoglobin level is a haptoglobin level that is higher, relative to a control haptoglobin level, by at least about 20%, 40%, 60%, 80%, 100%, or more. In some embodiments, the control haptoglobin level is a haptoglobin level in a subject suffering from PNH (or an average haptoglobin level in a population of subjects suffering from PNH) and not receiving the compstatin analog (e.g., a subject or population of subjects receiving a C5 inhibitor, e.g., eculizumab); a haptoglobin level in the subject (or an average haptoglobin level in the population of subjects) before receiving the compstatin analog; or a lower limit of a range of haptoglobin levels in a healthy subject. In some embodiments, a range of haptoglobin levels in a healthy subject is a gender-specific range. In some embodiments, the target haptoglobin level is achieved after about 1, 2, 3, 4, 5, 6, 7, 8, 9, 10, or more doses. In some embodiments, the target haptoglobin level is achieved after about 1 day, 1 week, 2 weeks, 3 weeks, 4 weeks, 6 weeks, 8 weeks, or more, of treatment. In some embodiments, the target haptoglobin level is sustained for about 1 day, 1 week, 2 weeks, 3 weeks, 4 weeks, 6 weeks, 8 weeks, 16 weeks, or more, following at least 1 dose of the compstatin analog described herein, e.g., pegcetacoplan. Haptoglobin levels can be assessed using standard methods known in the art.

In some embodiments, administration of a compstatin analog described herein, e.g., pegcetacoplan, to a subject (or to a population of subjects) reduces fatigue level in the subject (or reduces average fatigue level in the population of subjects) to a target fatigue level. In some embodiments, fatigue level is assessed using a FACIT-fatigue scale score. In some embodiments, the target fatigue level is a FACIT-fatigue scale score that is higher, relative to a control FACIT-fatigue scale score, by at least 5, 10, 15, 20, or more points. In some embodiments, the control FACIT-fatigue scale score is a FACIT-fatigue scale score in a subject suffering from PNH (or an average FACIT-fatigue scale score in a population of subjects) and not receiving the compstatin analog (e.g., a subject or population of subjects receiving a C5 inhibitor, e.g., eculizumab); a FACIT-fatigue scale score in the subject (or an average FACIT-fatigue score in the population of subjects) before receiving the compstatin analog; or a lower limit of a range of FACIT-fatigue scale scores in a healthy subject. In some embodiments, a range of FACIT-fatigue scale scores in a healthy subject is a gender-specific range. In some embodiments, the target FACIT-fatigue scale score is about 32, 34, 36, 38, 40, 42, 44, 46, or 48. In some embodiments, the target FACIT-fatigue scale score is achieved after about 1, 2, 3, 4, 5, 6, 7, 8, 9, 10, or more doses. In some embodiments, the target FACIT-fatigue scale score is achieved after about 1 day, 1 week, 2 weeks, 3 weeks, 4 weeks, 6 weeks, 8 weeks, or more, of treatment. In some embodiments, the target FACIT-fatigue scale score is sustained for about 1 day, 1 week, 2 weeks, 3 weeks, 4 weeks, 6 weeks, 8 weeks, 16 weeks, or more, following at least 1 dose of the compstatin analog described herein, e.g., pegcetacoplan.

The FACIT Fatigue Scale is an art-recognized 13 item Likert scaled instrument that is self-administered by subjects. Subject are presented with 13 statements and asked to indicate their responses as it applies to the past 7 days. The 5 possible responses are “Not at all” (0), “A little bit” (1), “Somewhat” (2), “Quite a bit” (3) and “Very much” (4). With 13 statements the total score has a range of 0 to 52. Before calculating the total score, some responses are reversed to ensure that the higher score corresponds to a higher quality of life.

In some embodiments, administration of a compstatin analog described herein, e.g., pegcetacoplan, to a subject (or to a population of subjects) increases one or more measures of quality of life in the subject (or increases the average of one or more measures of quality of life in the population of subjects) to a target level. In some embodiments, quality of life is assessed using a Linear Analog Scale Assessment (LASA) score and/or a Quality of Life Questionnaire (QLQ-C30) score. In some embodiments, the target level is a LASA score and/or a QLQ-C30 score that is higher, relative to a control LASA score and/or a control QLQ-C30 score, by at least 1, 2, 3, 4, 5, 10, 15, 20, 25, 30, 35, 40, 50 or more points. In some embodiments, the control LASA score or control QLQ-C30 score is a LASA score or QLQ-C30 score from a subject suffering from PNH (or an average LASA score or average QLQ-C30 score from a population of subjects suffering from PNH) and not receiving the compstatin analog (e.g., e.g., a subject or population of subjects receiving a C5 inhibitor, e.g., eculizumab); a LASA score or QLQ-C30 score from the subject (or an average LASA score or average QLQ-30 score from the population of subjects) before receiving the compstatin analog; or a lower limit of a range of LASA score or QLQ-C30 scores in a healthy subject. In some embodiments, a range of LASA or QLQ-30 scores in a healthy subject is a gender-specific range. In some embodiments, the target LASA score or QLQ-C30 score is achieved after about 1, 2, 3, 4, 5, 6, 7, 8, 9, 10, or more doses. In some embodiments, the target LASA score or QLQ-C30 score is achieved after about 1 day, 1 week, 2 weeks, 3 weeks, 4 weeks, 6 weeks, 8 weeks, 16 weeks, or more, of treatment. In some embodiments, the target LASA score or QLQ-C30 score is sustained for about 1 day, 1 week, 2 weeks, 3 weeks, 4 weeks, 6 weeks, 8 weeks, 16 weeks, or more, following at least 1 dose of the compstatin analog described herein, e.g., pegcetacoplan.

The Linear Analog Scale assessment (LASA) is known in the art and consists of three items asking respondents to rate their perceived level of functioning. Specific domains include activity level, ability to carry out daily activities, and an item for overall QOL. The EORTC QLQ-C30 questionnaire (version 3.0) is known in the art and consists of 30 questions comprised of both multi-item scales and single-item measures to assess overall quality of life in subjects. Questions are designated by functional scales, symptom scales, and global patient QOL/overall perceived health status. Scoring guidelines from EORTC can be used to calculate patients’ scores.

In some embodiments, one or more of the preceding parameters is measured before and/or after administration of a compstatin analog described herein, e.g., pegcetacoplan. For example, a compstatin analog described herein, e.g., pegcetacoplan, is administered twice weekly or every 3 days for about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years3, years, 4 years, 5 years, or longer, and one or more of the preceding parameters is measured before any treatment and/or after about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years, 3 years, 4 years, 5 years, or longer.

In some embodiments a subject (or a population of subjects) who has been or is being treated with eculizumab and continues to exhibit evidence of hemolysis, e.g., clinically significant hemolysis, such as causing anemia and/or requiring transfusion, is treated with a compstatin analog described herein (e.g., pegcetacoplan). In some embodiments, a subject (or population of subjects) who has been or is being treated with eculizumab and exhibits a hemoglobin level (or average hemoglobin level) of less than about 12 g/dL, e.g., less than about 11 g/dL, e.g., less than about 10.5 g/dL, e.g., less than about 10 g/dL, e.g., less than about 9 g/dL, e.g., less than about 8 g/dL, or less, is administered a compstatin analog described herein, e.g., pegcetacoplan, e.g., is administered twice weekly or every 3 days, at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg, for about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years, 3 years, 4 years, 5 years, or longer.

In some embodiments the subject (or population of subjects) has a platelet count (or an average platelet count) of at least 50,000 /mm³ and less than 100,000 /mm³ within 8 weeks prior to the subject’s (or prior to the population’s) first dose of pegcetacoplan. In some embodiments, the subject (or population of subjects) has a platelet count (or an average platelet count) of at least 100,000 /mm³ within 8 weeks prior to the subject’s (or prior to the population’s) first dose of pegcetacoplan. In some embodiments, the subject (or population of subjects) has received 1, 2, or 3 transfusions (or an average of 1, 2, or 3 transfusions) during the 12 months prior to the subject’s (or prior to the population’s) first dose of pegcetacoplan. In some embodiments, the subject (or population of subjects) has received at least 4 transfusions (or an average of at least 4 transfusions) during the 12 months prior to the subject’s first dose of pegcetacoplan.

In some embodiments, the subject (or population of subjects) has been under treatment with a C5 inhibitor, e.g., an anti-C5 antibody, e.g., eculizumab, over the 6 months prior to the subject’s (or prior to the population’s) first dose of pegcetacoplan. In some embodiments, the subject (or population of subjects) has been under treatment with a C5 inhibitor, e.g., an anti-C5 antibody, e.g., eculizumab, over the 12 months prior to the subject’s (or prior to the population’s) first dose of pegcetacoplan. In some embodiments, the subject (or population of subjects) has been under treatment with a C5 inhibitor, e.g., an anti-C5 antibody, e.g., eculizumab, over the 12 months prior to the subject’s (or prior to the population’s) first dose of pegcetacoplan and received 1, 2, or 3 transfusions (or received an average of 1, 2, or 3 transfusions) during said 12 month period. In some embodiments, the subject (or population of subjects) has been under treatment with a C5 inhibitor, e.g., an anti-C5 antibody, over the 12 months prior to the subject’s (or prior to the population’s) first dose of pegcetacoplan and received at least 4 transfusions (or received an average of at least 4 transfusions) during said 12 month period. In some embodiments, the C5 inhibitor is approved for treatment of PNH and treatment with the C5 inhibitor, e.g., anti-C5 antibody, was at an approved or recommended dosing regimen for treatment of PNH with the C5 inhibitor. (In some embodiments, an approved or recommended dosing regimen is on a label (package insert) as approved by a government agency responsible for regulating prescription drug products (e.g., the US Food & Drug Administration or the European Medicines Agency) and containing, among other things, prescribing information for a drug.) In some embodiments, treatment with the C5 inhibitor, e.g., anti-C5 antibody, was at a dosing regimen resulting in administration of a greater amount of the C5 inhibitor over time than an approved or recommended dosing regimen for treatment of PNH with the C5 inhibitor. For example, the dosing regimen for the C5 inhibitor may include administration of a higher maintenance dose and/or administration using a shorter dosing interval than an approved or recommended dosing regimen for treatment of PNH. For example, in some embodiments the subject (or population of subjects) may have been receiving doses of between about 25% (e.g., about 33%) and about 50% higher than the approved or recommended dose. For example, in some embodiments, the subject (or population of subjects) has been receiving 1200 mg eculizumab every other week.

For example, the approved standard regimen for eculizumab (brand name Soliris®) for treatment of PNH in patients 18 years of age and older is 600 mg weekly for the first 4 weeks, followed by 900 mg for the fifth dose 1 week later, then 900 mg every 2 weeks thereafter, administered by intravenous infusion. In some embodiments, a subject suffering from PNH is treated by subcutaneously administering to the subject about 1080 mg pegcetacoplan twice weekly or every three days for a 4-week period, during which the subject receives at least one dose of eculizumab. In some embodiments, after the 4-week period, the subject is subcutaneously administered about 1080 mg pegcetacoplan twice weekly or every three days, during which the subject does not receive any doses of eculizumab. In some embodiments, after the 4-week period, the subject receives at least one dose of eculizumab. In some embodiments, a subject suffering from PNH is treated by administering to the subject a pegcetacoplan dosing regimen comprising: (i) subcutaneously administering to the subject about 1080 mg pegcetacoplan twice weekly or every three days for a 4-week pegcetacoplan period; and (ii) subcutaneously administering to the subject about 1080 mg pegcetacoplan twice weekly or every three days after the 4-week pegcetacoplan period; wherein prior to or during the 4-week pegcetacoplan period, the subject received or is receiving an eculizumab dosing regimen comprising (a) weekly administration of a first amount (e.g., 600 mg) of eculizumab for 4 weeks; (b) administration of a second amount (e.g., 900 mg) of eculizumab one week later; and (c) administration of the second amount (e.g., 900 mg) of eculizumab every two weeks thereafter; and wherein after the 4-week pegcetacoplan period, the subject does not receive any doses of eculizumab. In some embodiments, the subject has been on treatment with eculizumab, e.g., every two weeks, for at least 3 months prior to first dose of pegcetacoplan.

The approved standard regimen for ravulizumab (also known as ravulizumab-cwvz; brand name ULTOMIRIS®) for treatment of PNH in patients 18 years and older consists of a loading dose followed by maintenance dosing, administered by intravenous infusion. Doses are to be administered based on the patient’s body weight, as shown in the table below. Starting 2 weeks after the loading dose administration, begin maintenance doses at a once every 8-week interval. The dosing schedule is allowed to occasionally vary within 7 days of the scheduled infusion day (except for the first maintenance dose of ULTOMIRIS®) but the subsequent dose should be administered according to the original schedule:

| Body Weight Range (kg) | Loading Dose (mg) | Maintenance Dose (mg) |
|---|---|---|
| greater than or equal to 40 to less than 60 | 2,400 | 3,000 |
| greater than or equal to 60 to less than 100 | 2,700 | 3,300 |
| greater than or equal to 100 | 3,000 | 3,600 |

In some embodiments, a subject suffering from PNH is treated by subcutaneously administering to the subject about 1080 mg pegcetacoplan twice weekly or every three days for a 4-week period, during which the subject receives at least one dose of ravulizumab. In some embodiments, after the 4-week period, the subject is subcutaneously administered about 1080 mg pegcetacoplan twice weekly or every three days, during which the subject does not receive any doses of ravulizumab. In some embodiments, after the 4-week period, the subject receives at least one dose of ravulizumab. In some embodiments, a subject suffering from PNH is treated by administering to the subject a pegcetacoplan dosing regimen comprising: (i) subcutaneously administering to the subject about 1080 mg pegcetacoplan twice weekly or every three days for a 4-week pegcetacoplan period; and (ii) subcutaneously administering to the subject about 1080 mg pegcetacoplan twice weekly or every three days after the 4-week pegcetacoplan period; wherein prior to or during the 4-week pegcetacoplan period, the subject received or is receiving a ravulizumab dosing regimen comprising (a) administration of a loading dose of a first concentration of ravulizumab (e.g., 2400 mg for a subject with body weight of greater than or equal to 40 kg to less than 60 kg; 2700 mg for a subject with body weight of greater than or equal to 60 kg to less than 100 kg; 3000 mg for a subject with body weight of greater than or equal to 100 kg); and (b) starting 2 weeks later, administration of a maintenance dose of a second concentration of ravulizumab (e.g., 3000 mg for a subject with body weight of greater than or equal to 40 kg to less than 60 kg; 3300 mg for a subject with body weight of greater than or equal to 60 kg to less than 100 kg; 3600 mg for a subject with body weight of greater than or equal to 100 kg) once every 8 weeks; and wherein after the 4-week pegcetacoplan period, the subject does not receive any doses of ravulizumab. In some embodiments, the subject has been receiving ravulizumab, e.g., according to an approved dosing regimen, for at least 3 months before the first dose of pegcetacoplan, and remains on such ravulizumab regimen for at least 4 weeks after the first dose of pegcetacoplan. In some embodiments, the subject has been receiving ravulizumab, e.g., weekly subcutaneously, for at least 3 months before the first dose of pegcetacoplan, and remains on such ravulizumab regimen for at least 4 weeks after the first dose of pegcetacoplan.

In some embodiments, the C5 inhibitor has a terminal half-life of between 6 and 8 weeks and the first dose of pegcetacoplan is administered within 4 weeks following the last dose of the C5 inhibitor. In some embodiments, the C5 inhibitor has an approved or recommended dosing interval of 8 weeks and the first dose of pegcetacoplan is administered within 4 weeks following the last dose of the C5 inhibitor.

In some embodiments, a subject (or a population of subjects) may be monitored for evidence of hemolysis after administration of pegcetacoplan. For example, the subject (or population of subjects) may be monitored by measuring the subject’s LDH level (or the average LDH level in the population of subjects). In some embodiments, a subject (or a population of subjects) may be monitored twice weekly, weekly, or every other week for at least a predetermined time period, e.g., at least 2 weeks, e.g., between 2 and 12 weeks, e.g., between 2 and 8 weeks, e.g., between 2 and 6 weeks, e.g., between 2 and 4 weeks, after the first dose of pegcetacoplan. In some embodiments wherein a subject (or a population of subjects) who has been receiving treatment with a C5 inhibitor is transitioned to treatment with pegcetacoplan, a subject (or population of subjects) may be monitored twice weekly, weekly, or every other week for at least a predetermined time period, e.g., at least 2 weeks, e.g., between 2 and 12 weeks, e.g., between 2 and 8 weeks, e.g., between 2 and 6 weeks, e.g., between 2 and 4 weeks, after the last dose of the C5 inhibitor.

In some embodiments, a subject may be identified as exhibiting hemolysis (e.g., acute hemolysis) if the subject exhibits a measured LDH level that is at least 2 × ULN. In some embodiments, a subject may be identified as exhibiting hemolysis (e.g., acute hemolysis) if the subject additionally exhibits at least one additional sign or symptom of hemolysis (e.g., decrease in hemoglobin (e.g., decrease of at least 1 g/dL or at least 2 g/dL, or decrease Hb below 10 g/dL), hemoglobinuria, or increased fatigue (e.g., an increase of at least 3 points on FACIT)). In some embodiments, a subject may be identified as exhibiting hemolysis (e.g., acute hemolysis) if such subject exhibits at least one new or worsening symptom or sign of hemolysis (e.g., fatigue, hemoglobinuria, abdominal pain, dysphagia, dyspnea, anemia (e.g., hemoglobin <10 grams (g)/deciliter (dL)), major adverse vascular event (including thrombosis), or erectile dysfunction) in the presence of elevated LDH ≥ 2 times the upper limit of normal (ULN). In some embodiments, a subject may be identified as exhibiting hemolysis (e.g., acute hemolysis) if such subject exhibits an LDH at least 2× ULN after having an LDH below a predetermined level, e.g., below 1.5 × ULN, for a period of time, e.g., at least 4 weeks, at least 8 weeks, at least 12 weeks.

In some embodiments, if the subject’s LDH level (or the average LDH level of the population of subjects) remains at or exceeds a specified level (e.g., 2 × ULN) after a selected time (e.g., after the subject or population of subjects has been treated with about 1080 mg pegcetacoplan twice weekly for 2, 3, or 4 weeks), the subject’s (or the population’s) dosing regimen may be changed to about 1080 mg pegcetacoplan every three days.

In some embodiments, after receiving about 1080 mg pegcetacoplan subcutaneously twice weekly for a period (e.g., at least 2, 3, 4, 6, 8, 10, 12 weeks or longer), a subject or population of subjects exhibits one or more signs of hemolysis (e.g., a measured LDH level that is at least 2 × ULN, a decrease in hemoglobin, hemoglobinuria, and/or fatigue), and the subject or population of subjects is selected for and/or is administered a modified dosing regimen of pegcetacoplan.

In some embodiments, after receiving an initial dosing regimen of about 1080 mg pegcetacoplan subcutaneously twice weekly for a first period (e.g., at least 2, 3, 4, 6, 8, 10, 12, 16, 20, 24, 36, 42, 48, 52 weeks, or longer), a subject does not exhibit a stable and/or average (e.g., over the first period) measured level of LDH below 2 × ULN (e.g., below 1.5 × ULN), and the modified dosing regimen of pegcetacoplan is 1080 mg pegcetacoplan subcutaneously every 3 days for a second period (e.g., at least 2, 3, 4, 6, 8, 10, 12, 16, 20, 24, 36, 42, 48, 52 weeks, or longer).

In some embodiments, after receiving an initial dosing regimen of about 1080 mg pegcetacoplan subcutaneously twice weekly for a first period (e.g., at least 2, 3, 4, 6, 8, 10, 12, 16, 20, 24, 36, 42, 48, 52 weeks, or longer) during which a subject exhibits a stable and/or average (e.g., over the first period) measured level of LDH below 2 × ULN (e.g., below 1.5 × ULN), the subject exhibits a measured LDH level that is at least 2 × ULN, and the modified dosing regimen of pegcetacoplan is (a) a single dose of about 1080 mg pegcetacoplan intravenously, or (b) about 1080 mg pegcetacoplan subcutaneously every 24 hours for 3 doses. In some embodiments, a subject may receive one or more additional courses of a modified dosing regimen (e.g., that are the same or are different from the initial modified dosing regimen), separated by, e.g., at least 7, 14, or 21 days. In some embodiments, after receiving the modified dosing regimen (or courses of modified dosing regimen), the subject no longer receives the modified dosing regimen but subsequently receives the initial dosing regimen of about 1080 mg pegcetacoplan subcutaneously twice weekly.

In some embodiments, after receiving an initial dosing regimen of about 1080 mg pegcetacoplan subcutaneously twice weekly for a first period (e.g., at least 2, 3, 4, 6, 8, 10, 12, 16, 20, 24, 36, 42, 48, 52 weeks, or longer), during which a subject exhibits a stable and/or average (e.g., over the first period) measured level of LDH below 2 × ULN (e.g., below 1.5 × ULN), the subject exhibits a measured LDH level that is at least 2 × ULN, and the modified dosing regimen of pegcetacoplan is a single dose of about 1080 mg pegcetacoplan intravenously, followed by about 1080 mg pegcetacoplan subcutaneously every three days (e.g., at least 2, 3, 4, 6, 8, 10, 12, 16, 20, 24, 36, 42, 48, 52 weeks, or longer). In some embodiments, after receiving about 1080 mg pegcetacoplan subcutaneously every three days for at least 2, 3, 4, 6, 8, 10, 12, 16, 20, 24, 36, 42, 48, 52 weeks, or longer, the subject no longer receives about 1080 mg pegcetacoplan subcutaneously every three days, but subsequently receives the initial dosing regimen of about 1080 mg pegcetacoplan subcutaneously twice weekly.

In some embodiments, after receiving an initial dosing regimen of about 1080 mg pegcetacoplan subcutaneously twice weekly for a first period (e.g., at least 2, 3, 4, 6, 8, 10, 12, 16, 20, 24, 36, 42, 48, 52 weeks, or longer), during which a subject exhibits a stable and/or average (e.g., over the first period) measured level of LDH below 2 × ULN (e.g., below 1.5 × ULN), the subject exhibits a measured LDH level that is at least 2 × ULN, and the modified dosing regimen of pegcetacoplan is about 1080 mg pegcetacoplan subcutaneously every 24 hours for 3 doses, followed by about 1080 mg pegcetacoplan subcutaneously every three days (e.g., at least 2, 3, 4, 6, 8, 10, 12, 16, 20, 24, 36, 42, 48, 52 weeks, or longer). In some embodiments, after receiving about 1080 mg pegcetacoplan subcutaneously every three days for at least 2, 3, 4, 6, 8, 10, 12, 16, 20, 24, 36, 42, 48, 52 weeks, or longer, the subject no longer receives about 1080 mg pegcetacoplan subcutaneously every three days, but subsequently receives the initial dosing regimen of about 1080 mg pegcetacoplan subcutaneously twice weekly.

In some embodiments, after receiving about 1080 mg pegcetacoplan subcutaneously twice weekly for a period (e.g., 2, 3, 4, 6, 8, 10, 12 weeks or longer), a subject or population of subjects exhibits one or more signs of hemolysis (e.g., a measured LDH level that is at least 2 × ULN, a decrease in hemoglobin, hemoglobinuria, and/or fatigue), and the subject or population of subjects is selected for and/or is administered a single dose of about 1080 mg pegcetacoplan intravenously, followed by about 1080 mg pegcetacoplan subcutaneously every three days. In some embodiments, after receiving about 1080 mg pegcetacoplan subcutaneously twice weekly for a period (e.g., 2, 3, 4, 6, 8, 10, 12 weeks or longer), a subject or population of subjects exhibits one or more signs of hemolysis (e.g., a measured LDH level that is at least 2 × ULN, a decrease in hemoglobin, hemoglobinuria, and/or fatigue), and the subject or population of subjects is selected for and/or is administered about 1080 mg pegcetacoplan subcutaneously every 24 hours for 3 doses, followed by about 1080 mg pegcetacoplan subcutaneously every three days.

In some embodiments, after receiving about 1080 mg pegcetacoplan subcutaneously twice weekly for a period (e.g., at least 2, 3, 4, 6, 8, 10, 12 weeks or longer) in combination with a C5 inhibitor (e.g., an anti-C5 antibody, e.g., eculizumab), a subject or population of subjects exhibits one or more signs of hemolysis (e.g., a measured LDH level that is at least 2 × ULN, a decrease in hemoglobin, hemoglobinuria, and/or fatigue), and the subject or population of subjects is selected for and/or is administered a modified dosing regimen of pegcetacoplan. For example, in some embodiments, after receiving about 1080 mg pegcetacoplan subcutaneously twice weekly for a period (e.g., 2, 3, 4, 6, 8, 10, 12 weeks or longer) in combination with a C5 inhibitor (e.g., an anti-C5 antibody, e.g., eculizumab), a subject or population of subjects exhibits one or more signs of hemolysis (e.g., a measured LDH level that is at least 2 × ULN, a decrease in hemoglobin, hemoglobinuria, and/or fatigue), and the subject or population of subjects is selected for and/or is administered a single dose of about 1080 mg pegcetacoplan intravenously, followed by about 1080 mg pegcetacoplan subcutaneously every three days. In some embodiments, after receiving about 1080 mg pegcetacoplan subcutaneously twice weekly for a period (e.g., 2, 3, 4, 6, 8, 10, 12 weeks or longer) in combination with a C5 inhibitor (e.g., an anti-C5 antibody, e.g., eculizumab), a subject or population of subjects exhibits one or more signs of hemolysis (e.g., a measured LDH level that is at least 2 × ULN, a decrease in hemoglobin, hemoglobinuria, and/or fatigue), and the subject or population of subjects is selected for and/or is administered about 1080 mg pegcetacoplan subcutaneously every 24 hours for 3 doses, followed by about 1080 mg pegcetacoplan subcutaneously every three days.

In some embodiments, after receiving about 1080 mg pegcetacoplan subcutaneously every 3 days for a period (e.g., at least 2, 3, 4, 6, 8, 10, 12 weeks or longer), a subject or population of subjects exhibits one or more signs of hemolysis (e.g., a measured LDH level that is at least 2 × ULN, a decrease in hemoglobin, hemoglobinuria, and/or fatigue), and the subject or population of subjects is selected for and/or is administered a modified dosing regimen of pegcetacoplan.

In some embodiments, after receiving an initial dosing regimen of about 1080 mg pegcetacoplan subcutaneously every 3 days for a first period (e.g., at least 2, 3, 4, 6, 8, 10, 12, 16, 20, 24, 36, 42, 48, 52 weeks, or longer), a subject does not exhibit a stable and/or average (e.g., over the first period) measured level of LDH below 2 × ULN (e.g., below 1.5 × ULN), and the modified dosing regimen of pegcetacoplan is 1080 mg pegcetacoplan subcutaneously thrice weekly for a second period (e.g., at least 2, 3, 4, 6, 8, 10, 12, 16, 20, 24, 36, 42, 48, 52 weeks, or longer).

In some embodiments, after receiving an initial dosing regimen of about 1080 mg pegcetacoplan subcutaneously every 3 days for a first period (e.g., at least 2, 3, 4, 6, 8, 10, 12, 16, 20, 24, 36, 42, 48, 52 weeks, or longer) during which a subject exhibits a stable and/or average (e.g., over the first period) measured level of LDH below 2 × ULN (e.g., below 1.5 × ULN), the subject exhibits a measured LDH level that is at least 2 × ULN, and the modified dosing regimen of pegcetacoplan is (a) a single dose of about 1080 mg pegcetacoplan intravenously, or (b) about 1080 mg pegcetacoplan subcutaneously every 24 hours for 3 doses. In some embodiments, a subject may receive one or more additional courses of a modified dosing regimen (e.g., that are the same or are different from the initial modified dosing regimen), separated by, e.g., at least 7, 14, or 21 days. In some embodiments, after receiving the modified dosing regimen (or courses of modified dosing regimen), the subject no longer receives the modified dosing regimen but subsequently receives the initial dosing regimen of about 1080 mg pegcetacoplan subcutaneously every 3 days.

In some embodiments, after receiving an initial dosing regimen of about 1080 mg pegcetacoplan subcutaneously every 3 days for a first period (e.g., at least 2, 3, 4, 6, 8, 10, 12, 16, 20, 24, 36, 42, 48, 52 weeks, or longer), during which a subject exhibits a stable and/or average (e.g., over the first period) measured level of LDH below 2 × ULN (e.g., below 1.5 × ULN), the subject exhibits a measured LDH level that is at least 2 × ULN, and the modified dosing regimen of pegcetacoplan is a single dose of about 1080 mg pegcetacoplan intravenously, followed by about 1080 mg pegcetacoplan subcutaneously thrice weekly (e.g., at least 2, 3, 4, 6, 8, 10, 12, 16, 20, 24, 36, 42, 48, 52 weeks, or longer). In some embodiments, after receiving about 1080 mg pegcetacoplan subcutaneously thrice weekly for at least 2, 3, 4, 6, 8, 10, 12, 16, 20, 24, 36, 42, 48, 52 weeks, or longer, the subject no longer receives about 1080 mg pegcetacoplan subcutaneously thrice weekly, but subsequently receives the initial dosing regimen of about 1080 mg pegcetacoplan subcutaneously every 3 days.

In some embodiments, after receiving an initial dosing regimen of about 1080 mg pegcetacoplan subcutaneously every 3 days for a first period (e.g., at least 2, 3, 4, 6, 8, 10, 12,16, 20, 24, 36, 42, 48, 52 weeks, or longer), during which a subject exhibits a stable and/or average (e.g., over the first period) measured level of LDH below 2 × ULN (e.g., below 1.5 × ULN), the subject exhibits a measured LDH level that is at least 2 × ULN, and the modified dosing regimen of pegcetacoplan is about 1080 mg pegcetacoplan subcutaneously every 24 hours for 3 doses, followed by about 1080 mg pegcetacoplan subcutaneously thrice weekly (e.g., at least 2, 3, 4, 6, 8, 10, 12, 16, 20, 24, 36, 42, 48, 52 weeks, or longer). In some embodiments, after receiving about 1080 mg pegcetacoplan subcutaneously thrice weekly for at least 2, 3, 4, 6, 8, 10, 12, 16, 20, 24, 36, 42, 48, 52 weeks, or longer, the subject no longer receives about 1080 mg pegcetacoplan subcutaneously thrice weekly, but subsequently receives the initial dosing regimen of about 1080 mg pegcetacoplan subcutaneously every 3 days.

In some embodiments, after receiving about 1080 mg pegcetacoplan subcutaneously every 3 days for a period (e.g., 2, 3, 4, 6, 8, 10, 12 weeks or longer), a subject or population of subjects exhibits one or more signs of hemolysis (e.g., a measured LDH level that is at least 2 × ULN, a decrease in hemoglobin, hemoglobinuria, and/or fatigue), and the subject or population of subjects is selected for and/or is administered a single dose of about 1080 mg pegcetacoplan intravenously, followed by about 1080 mg pegcetacoplan subcutaneously thrice weekly. In some embodiments, after receiving about 1080 mg pegcetacoplan subcutaneously every 3 days for a period (e.g., 2, 3, 4, 6, 8, 10, 12 weeks or longer), a subject or population of subjects exhibits one or more signs of hemolysis (e.g., a measured LDH level that is at least 2 × ULN, a decrease in hemoglobin, hemoglobinuria, and/or fatigue), and the subject or population of subjects is selected for and/or is administered about 1080 mg pegcetacoplan subcutaneously every 24 hours for 3 doses, followed by about 1080 mg pegcetacoplan subcutaneously thrice weekly.

In some embodiments, after receiving about 1080 mg pegcetacoplan subcutaneously every 3 days for a period (e.g., 2, 3, 4, 6, 8, 10, 12 weeks or longer) in combination with a C5 inhibitor (e.g., an anti-C5 antibody, e.g., eculizumab), a subject or population of subjects exhibits one or more signs of hemolysis (e.g., a measured LDH level that is at least 2 × ULN, a decrease in hemoglobin, hemoglobinuria, and/or fatigue), and the subject or population of subjects is selected for and/or is administered a modified dosing regimen of pegcetacoplan. For example, in some embodiments, after receiving about 1080 mg pegcetacoplan subcutaneously every 3 days for a period (e.g., 2, 3, 4, 6, 8, 10, 12 weeks or longer) in combination with a C5 inhibitor (e.g., an anti-C5 antibody, e.g., eculizumab), a subject or population of subjects exhibits one or more signs of hemolysis (e.g., a measured LDH level that is at least 2 × ULN, a decrease in hemoglobin, hemoglobinuria, and/or fatigue), and the subject or population of subjects is selected for and/or is administered a single dose of about 1080 mg pegcetacoplan intravenously, followed by about 1080 mg pegcetacoplan subcutaneously thrice weekly. In some embodiments, after receiving about 1080 mg pegcetacoplan subcutaneously every 3 days for a period (e.g., 2, 3, 4, 6, 8, 10, 12 weeks or longer) in combination with a C5 inhibitor (e.g., an anti-C5 antibody, e.g., eculizumab), a subject or population of subjects exhibits one or more signs of hemolysis (e.g., a measured LDH level that is at least 2 × ULN, a decrease in hemoglobin, hemoglobinuria, and/or fatigue), and the subject or population of subjects is selected for and/or is administered about 1080 mg pegcetacoplan subcutaneously every 24 hours for 3 doses, followed by about 1080 mg pegcetacoplan subcutaneously thrice weekly.

In some embodiments, a subject (or a population of subjects) who is diagnosed with PNH and who has previously been treated with eculizumab is administered a compstatin analog described herein, e.g., pegcetacoplan, e.g., twice weekly or every 3 days, at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg, for about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years, 3 years, 4 years, 5 years, or longer, and is not administered eculizumab following the first dose of the compstatin analog, e.g., pegcetacoplan. In some embodiments, after treatment with eculizumab and before treatment with the compstatin analog, e.g., pegcetacoplan, the subject exhibits a hemoglobin level (or the population of subjects exhibits an average hemoglobin level) of less than about 12 g/dL, e.g., less than about 11 g/dL, e.g., less than about 10.5 g/dL, e.g., less than about 10 g/dL, e.g., less than about 9 g/dL, e.g., less than about 8 g/dL.

In some embodiments, a subject (or a population of subjects) who is diagnosed with PNH and who has not been treated with eculizumab is administered a compstatin analog described herein, e.g., pegcetacoplan, e.g., twice weekly or every 3 days, at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg, for about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years, 3 years, 4 years, 5 years, or longer.

In some embodiments, the age of the subject (or the population of subjects) is 18 years or greater (e.g., greater than 18); and/or the subject exhibits a hemoglobin level (or the population of subjects exhibits an average hemoglobin level) of less than about 12 g/dL, e.g., less than about 11 g/dL, e.g., less than about 10.5 g/dL, e.g., less than about 10 g/dL, e.g., less than about 9 g/dL, e.g., less than about 8 g/dL; and/or the subject has an absolute reticulocyte count (or the population of subjects has an average absolute reticulocyte count) of greater than the upper limit of normal, e.g., greater than 100, 110, 120, 130, 140, or 150 × 10⁹ /L); and/or the subject exhibits a platelet count (or the population of subjects exhibits an average platelet count) of greater than about 30,000 /mm³, greater than about 40,000 /mm³, greater than about 50,000 /mm³, greater than about 60,000 /mm³, or greater than about 70,000 /mm³; and/or the subject exhibits an absolute neutrophil count (or the population of subjects exhibits an average absolute neutrophil count) of greater than about 200 /mm³, greater than about 300 /mm³, greater than about 400 /mm³, greater than about 500 /mm³, greater than about 600 /mm³, or greater than about 700 /mm³; and/or the subject has a BMI of (or the population of subjects has an average BMI of) less than about 40 kg/m², less than about 37.5 kg/m², less than about 35 kg/m², less than about 32.5 kg/m², or less than about 30 kg/m²; and/or the subject (or the population of subjects) does not have a bacterial infection; and/or the subject (or the population of subjects) is not receiving iron, folic acid, vitamin B12, or EPO (though in some embodiments the subject is receiving one of these at a stable dose); and/or the subject (or the population of subjects) does not have hereditary complement deficiency or history of bone marrow transplantation; and/or the subject (or the population of subjects) does not exhibit significant cardiovascular instability or risk; and/or the subject (or the population of subjects) is not pregnant or breastfeeding; and the subject (or the population of subjects) is administered, e.g., subcutaneously, a compstatin analog described herein, e.g., pegcetacoplan, e.g., twice weekly or every 3 days, at a dosage of about 800 mg to about 1200 mg, e.g., about 1080 mg, for about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years, 3 years, 4 years, 5 years, or longer.

In some embodiments, the age of the subject (or the population of subjects) having or at risk of developing PNH is under 18 years (e.g., less than 18 (e.g., 12 to 17 years)), and a dosing regimen described herein for adults can be modified to achieve a similar dosing level of pegcetacoplan for a subject under 18 years of age. For example, in some embodiments, a compstatin analog described herein, e.g., pegcetacoplan, is administered, e.g., subcutaneously, to a pediatric subject (or a population of pediatric subjects) (e.g., 12-17 years of age, with a body weight of about 20-34 kg) at a dosage of about 300 mg to about 750 mg (e.g., about 400 mg to about 650 mg, e.g., about 500 mg to about 600 mg, e.g., about 540 mg) twice weekly for two initial doses followed by a dosage of about 400 mg to about 850 mg (e.g., about 500 mg to about 750 mg, e.g., about 600 mg to about 700 mg, e.g., about 648 mg) twice weekly. In some embodiments, a compstatin analog described herein, e.g., pegcetacoplan, is administered, e.g., subcutaneously, to a pediatric subject (or a population of pediatric subjects) (e.g., 12-17 years of age, with a body weight of about 35-49 kg) at a single dose of about 400 mg to about 850 mg (e.g., about 500 mg to about 750 mg, e.g., about 600 mg to about 700 mg, e.g., about 648 mg) followed by a dosage of about 500 mg to about 1000 mg (e.g., about 600 mg to about 900 mg, e.g., about 700 mg to about 850 mg, e.g., about 810 mg) twice weekly. In some embodiments, a compstatin analog described herein, e.g., pegcetacoplan, is administered, e.g., subcutaneously, to a pediatric subject (or a population of pediatric subjects) (e.g., 12-17 years of age, with a body weight of about 20-34 kg) at a dosage of about 400 mg to about 850 mg (e.g., about 500 mg to about 750 mg, e.g., about 600 mg to about 700 mg, e.g., about 648 mg) every 3 days. In some embodiments, a compstatin analog described herein, e.g., pegcetacoplan, is administered, e.g., subcutaneously, to a pediatric subject (or a population of pediatric subjects) (e.g., 12-17 years of age, with a body weight of about 35-49 kg) at a dosage of about 500 mg to about 1000 mg (e.g., about 600 mg to about 900 mg, e.g., about 700 mg to about 850 mg, e.g., about 810 mg) every 3 days.

In some embodiments, after treatment with compstatin analog described herein, e.g., pegcetacoplan (e.g., about 1080 mg subcutaneously twice weekly or every 3 days) for about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years, 3 years, 4 years, 5 years, or longer, a subject’s hemoglobin level, number of transfusions, number of PRBC units transfused, number of reticulocytes in the blood, level of LDH, level of indirect bilirubin, haptoglobin level, fatigue level, FACIT-fatigue scale score, LASA score, and/or QLQ-C30 score, is compared to a corresponding target level described herein (e.g., target hemoglobin level, target number of transfusions, target number of PRBC units transfused, target number of reticulocytes in the blood, target level of LDH, target level of indirect bilirubin, target haptoglobin level, target fatigue level, target FACIT-fatigue scale score, target LASA score, and/or target QLQ-C30 score, respectively). In some embodiments, after the comparison, a determination can be made about treatment or administration. For example, a determination can be made that treatment should continue or cease, that the dosage should be increased or decreased, that the dosing regimen should be altered. In some embodiments, a determination is made to switch from one therapy to another, e.g., to switch from treatment with an anti-C5 agent (e.g., an anti-C5 antibody, e.g., eculizumab), to a compstatin analog described herein, e.g., pegcetacoplan.

Methods described herein can include preparing and/or providing a report, such as in electronic, web-based, or paper form. The report can include one or more outputs from a method described herein, e.g., a subject’s response to a treatment described herein. In some embodiments, a report is generated, such as in paper or electronic form, which identifies one or more endpoints described herein (e.g., hemoglobin level, number of transfusions, number of PRBC units transfused, number of reticulocytes in the blood, level of LDH, level of indirect bilirubin, haptoglobin level, fatigue level, FACIT-fatigue scale score, LASA score, and/or QLQ-C30 score) for a subject, and optionally, a recommended course of therapy. In some embodiments, the report includes an identifier for the subject. In some embodiments, the report is in web-based form.

In some embodiments, additionally or alternatively, a report includes information on prognosis, resistance, or potential or suggested therapeutic options. The report can include information on the likely effectiveness of a therapeutic option, the acceptability of a therapeutic option, or the advisability of applying the therapeutic option to a subject, e.g., identified in the report. For example, the report can include information, or a recommendation, on the administration of a compstatin analog described herein, e.g., pegcetacoplan, and/or one or more C5 inhibitors (e.g., anti-C5 antibody, e.g., eculizumab) to the subject. The report can be delivered, e.g., to an entity described herein, within 7, 14, 21, 30, or 45 days from performing a method described herein.

In some embodiments, a report is generated to memorialize each time a subject is assessed using a method described herein. The subject can be reevaluated at intervals, such as every month, every two months, every six months or every year, or more or less frequently, to monitor the subject for responsiveness to compstatin analog, e.g., pegcetacoplan, and/or one or more C5 inhibitors (e.g., anti-C5 antibody, e.g., eculizumab) and/or for an improvement in one or more PNH symptoms, e.g., described herein. In some embodiments, the report can record at least the treatment history of the subject.

In some embodiments, the method further includes providing a report to another party. The other party can be, for example, the subject, a caregiver, a physician, an oncologist, a hospital, clinic, third-party payor, insurance company or a government office.

### IV. C5 Inhibitors

In some embodiments, a subject treated with a compstatin analog described herein, e.g., pegcetacoplan, has received one or more C5 inhibitors before treatment with the compstatin analog, receives one or more C5 inhibitors in combination with at least one dose of the compstatin analog, and/or continues to receive one or more C5 inhibitors during the entire treatment with the compstatin analog.

C5 inhibitors are known and/or commercially available. Non-limiting examples of C5 inhibitors include, e.g., eculizumab, ALXN1210 (ravulizumab), SKY59 (crovalimab), LFG316, REGN3918, ABP959, RA101495, Coversin, and ALNCC5 (described in, e.g., Risitano et al., Frontiers Immunology 10:1157 (2019)). Additional C5-targeting agents are described in, e.g., U.S. Pat. Nos. 9,718,880 and 9,079,949; and PCT Publs. WO2004106369; WO2010015608; WO2013093762; WO/2014/160129; WO2015134894; WO2015191951; WO/2016/040589; WO/2016/044419; WO2016098356; WO2016117346; WO2016123371; WO/2016/201301; WO2017104779; WO2017105939; WO2017212375; WO2017212391; WO/2017/214518; WO2017/217524; WO2017218515; WO2018106859; WO2018143266; WO2018165062; WO2018183449; WO2019014360; WO2019023564; WO2019084438; WO2019112984; WO2019118556; and WO2020006266.

In some embodiments, the C5 inhibitor is an anti-C5 antibody, e.g., an anti-C5 monoclonal antibody. In some embodiments, a C5 inhibitor is eculizumab or ravulizumab. In some embodiments, a C5 inhibitor is an antibody that binds to the same epitope as eculizumab or ravulizumab. In some embodiments, a C5 inhibitor is an antibody that competes for binding to C5 with eculizumab or ravulizumab. In some embodiments, a C5 inhibitor includes the same or substantially the same amino acid sequence as eculizumab or ravulizumab, or an antigen binding portion thereof.

In some embodiments, the disclosure provides methods of switching a subject from treatment with a C5 inhibitor, e.g., an anti-C5 monoclonal antibody, e.g., eculizumab or ravulizumab, to treatment with pegcetacoplan, e.g., according to a dosing regimen described herein.

### V. Pharmaceutical Compositions

A compstatin analog described herein, e.g., pegcetacoplan, can be incorporated into a pharmaceutical composition. Such pharmaceutical compositions are useful for, among other things, administration and delivery to a subject in vivo or ex vivo. In some embodiments, pharmaceutical compositions also contain a pharmaceutically acceptable carrier or excipient. Such excipients include any pharmaceutical agent, e.g., a pharmaceutical agent that does not itself induce an immune response harmful to the individual receiving the composition, and which may be administered without undue toxicity. As used herein the terms “pharmaceutically acceptable” and “physiologically acceptable” mean a biologically acceptable formulation, gaseous, liquid or solid, or mixture thereof, which is suitable for one or more routes of administration, in vivo delivery or contact. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

Pharmaceutical compositions may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In some embodiments, a pharmaceutical composition may be a lyophilized powder.

Pharmaceutical compositions can include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration can comprise aqueous and non-aqueous solutions, suspensions or emulsions of the active compound, which preparations are typically sterile and can be isotonic with the blood of the intended recipient. Non-limiting illustrative examples include water, buffered saline, Hanks’ solution, Ringer’s solution, dextrose, fructose, ethanol, animal, vegetable or synthetic oils. Aqueous injection suspensions may contain substances which increase the viscosity of the suspension, such as sodium carboxymethyl cellulose, sorbitol, or dextran. Additionally, suspensions of the active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility to allow for the preparation of highly concentrated solutions.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling can include amount, frequency, and method of administration.

Pharmaceutical compositions and delivery systems appropriate for the compositions, methods and uses of the disclosure are known in the art (see, e.g., Remington: The Science and Practice of Pharmacy. 21st Edition. Philadelphia, PA. Lippincott Williams & Wilkins, 2005).

A compstatin analog described herein, e.g., pegcetacoplan, can be administered by any suitable route. The route and/or mode of administration can vary depending upon the desired results. Methods and uses of the disclosure include delivery and administration systemically, regionally or locally, or by any route, for example, by injection or infusion. The mode of administration is left to the discretion of the practitioner. Delivery of a pharmaceutical composition in vivo may generally be accomplished via injection using a conventional syringe, although other delivery methods such as convection-enhanced delivery can also be used (see, e.g., U.S. Pat. No. 5,720,720). For example, compositions may be delivered subcutaneously, epidermally, epidurally, intracerebrally, intradermally, intranasally, intrathecally, intraorbitally, intramucosally, intraperitoneally, intravenously, intra-pleurally, subretinally, intraarterially, sublingually, intrahepatically, via the portal vein, and intramuscularly. In some embodiments, administration is via intravenous infusion, e.g., central or peripheral intravenous infusion. Other modes of administration include oral and pulmonary administration, suppositories, and transdermal applications. A clinician may determine the optimal route for administration.

In some embodiments a composition as described herein, and e.g., a composition comprising a compstatin analog described herein, e.g., pegcetacoplan, is administered using a device that delivers a dose of a pharmaceutical composition by injection, in some embodiments in an at least partly automated fashion upon activation. Such a device is referred to in the art as a “pen” or “autoinjector”, and these terms are used interchangeably herein. In general, a pen or autoinjector allows for injecting a dose of pharmaceutical composition contained in a cartridge, reservoir, or syringe through an automatically or manually inserted hypodermic needle(s) or through a high velocity jet. It may be designed for administration of a single dose or multiple doses.

In some embodiments, such a pen or autoinjector is utilized for intramuscular and/or subcutaneous injection. In accordance with the present disclosure, a pen or other autoinjector may be particularly useful for embodiments that utilize subcutaneous injection. Pens are typically devices that contain (or can be loaded with) a medication in a self-contained cartridge or reservoir and to which a needle can be attached.

In some embodiments, such injection is achieved by use of a pen (e.g., that may have been pre-loaded with an appropriate dose or volume). Pens can be durable (and reusable) or disposable. A durable pen typically uses a replaceable cartridge, which is disposed of when empty, and a new one is inserted in the pen. A disposable pen typically comes pre-filled with a medication in a cartridge or reservoir. When the cartridge or reservoir is empty, the pen can be discarded. The cartridge or reservoir may contain a single dose or multiple doses. To use a pen, a needle can be attached to the pen and inserted into the skin. Typically, a button can be pushed to administer a dose though in some embodiments other activation methods may be used. In some embodiments, an autoinjector may comprise a spring-loaded syringe, though one of ordinary skill in the art will appreciate that a variety of technologies are available to afford automatic administration. In some embodiments, by pressing a button or otherwise activating the device, the needle can be automatically inserted, and the medication can be delivered. In some embodiments, an autoinjector may be designed to insert the needle automatically and/or accurately to a desired depth in the subcutaneous tissue. A pen or autoinjector may comprise means such as a dial that allows a user to select or adjust a dose or injection depth.

In some embodiments, a composition as described herein, e.g., a compstatin analog described herein, e.g., pegcetacoplan, is administered using a device comprising a dual chamber syringe. Dry drug (e.g., lyophilized) is contained in one chamber. The second chamber contains a suitable pharmaceutically acceptable carrier. In order to use the device, the drug is first reconstituted by mixing the contents of the chambers. This can be accomplished in various ways, as is known in the art. In some embodiments, pushing the plunger causes the contents of the chambers to mix, e.g., by transferring the carrier into the chamber containing the lyophilized drug.

Thus a variety of drug delivery devices comprising a composition as described herein (e.g., a compstatin analog described herein, e.g., pegcetacoplan) may be provided e.g., prefilled syringes, dual chamber syringes, durable and/or disposable pens, and cartridges suitable for use with a pen. Such devices may contain one or more doses (e.g., one or more of any of the dose amounts described herein).

In certain embodiments a compstatin analog may be administered, e.g., subcutaneously, using a drug delivery device (sometimes referred to simply as a “delivery device”) that comprises a pump to introduce a liquid composition comprising the compstatin analog into the subject’s body. As will be appreciated, a pump may be any device that moves fluids by mechanical action as opposed to a conventional manually actuated syringe characterized in that the individual administering the medication (e.g., a health care provider or a subject who self-administers the medication) must directly depress a plunger into a barrel containing medication in order to effect the injection. It will be appreciated that a pump may be powered electrically or mechanically, e.g., as described herein. In some aspects, a delivery device comprising a pump may allow for convenient administration of doses according to a dosing regimen described herein.

In certain embodiments, the delivery device is portable. A portable device, also referred to as an “ambulatory” device, can be sufficiently light in weight and have appropriate dimensions so as to permit the subject to move about freely while the device is in use. In certain embodiments, such device does not require attachment to a pole or power outlet. In some embodiments a portable delivery device may be attached to a belt or shoulder strap or worn in a case that may be attached to a belt or shoulder strap, or may be placed in a pocket of a garment.

One of ordinary skill in the art appreciates that a pump may operate in any of a variety of ways and may utilize a variety of energy sources, e.g., disposable or rechargeable batteries, alternating current power supply (e.g., via a wall socket in a building), compressed gas, or energy stored in a compressed spring or in a stretched expandable resilient chamber. A device in which fluid is held in a stretchable balloon reservoir, and pressure from the elastic walls of the balloon reservoir drives fluid delivery may be referred to as an “elastomeric infusion pump”.

In some embodiments, a delivery device comprises a pump and a syringe containing a liquid to be administered and removably associated with the device, and a driving unit, which may be electronically controlled by a controller, arranged to make the plunger of the syringe slide so as to cause infusion of the liquid directly or via flexible tubing through a piercing member such as a needle or cannula that is introduced into the subject’s body under the skin. For example, in some embodiments a pump may comprise a motor that turns a screw that pushes the plunger on a syringe that contains the liquid. Pushing of the plunger causes liquid to be expelled from the syringe and introduced into the subject’s body via an attached piercing member. Exemplary pumps are described in, e.g., U.S. Pat. Nos. 6,447,487; 6,592,551; 6,645,177; 8,187,228; U.S. Pat. Application Publication Nos. 20020123740, 20030229311, 20060184123, 20070100281, 20090123309, 20150038906. The Crono PID (NDC No.: 8423.2000.02), Crono S-PID30, and Crono S-PID 50 (NDC No.: 8423.2000.04) (Canè s.r.l. Medical Technology (Rivoli, Italy)), and the T34™ Ambulatory Syringe Pump and the T60™ Ambulatory Syringe Pump (CME Medical, Blackpool, UK) are exemplary portable syringe infusion pumps that may be used in certain embodiments.

In some embodiments the pump may be electronically programmable or controlled. In some embodiments the pump is not electronically programmable or controlled.

In some embodiments a pump uses electricity as a source of power. In some embodiments a pump does not use electricity as a source of power. Such a pump may, for example, use a compressed spring or compressed gas as an energy source.

In some embodiments the pump is a constant-pressure pump that applies a constant pressure to depress the barrel of a syringe containing the liquid to be administered. An example of a constant-pressure pump is the Freedom60® infusion system (RMS Medical Products, Chester, NY). In some embodiments a FreedomEdge ® infusion system (RMS Medical Products) may be used, e.g., with a syringe capable of holding up to 20 ml or a syringe capable of holding up to 30 ml. Another example of a constant pressure device is the SCIg60 syringe pump (EMED Technologies, El Dorado Hills, CA). In some embodiments a valve may control the flow rate of the liquid. In some embodiments tubing connected to the syringe may control the flow rate of the liquid, e.g., as described in U.S. Pat. Application Nos. 20150374911 and/or 20160256625. In some embodiments a delivery rate of between 0.5 ml/minute and 1 ml/minute may be used.

In some embodiments the liquid to be administered is contained in a pressurized chamber prior to administration. In some embodiments the liquid is contained in a resilient, expandable container portion such as a bladder or balloon prior to delivery. The expandable container portion may be made of or comprise an inner lining of compatible medical grade butyl, silicone or other material suitable for holding the liquid. The container portion expands upon filling with liquid (e.g., with a unit dose of the compound to be administered), so as to exert pressure on the liquid. One of ordinary skill in the art appreciates that the container portion may be filled in a variety of ways. In some embodiments filling of the expandable container portion may be accomplished manually, e.g., using a manually actuated syringe, or may be performed using a filling apparatus. After the device is attached to the subject’s skin, a piercing member such as a needle or cannula, which may be spring loaded, may automatically or following additional activation, such as by pressing a button, emerge from the device’s housing and pierce the skin. Subsequently, either automatically or following additional activation, such as by pressing a button, pressure forces the liquid out of the chamber or container and into the subject’s body via the needle or cannula. Exemplary devices are described in U.S. Pat. Application Pub. Nos. 20130018326, and/or 20150217058.

In some embodiments the delivery device is an “on-body delivery device”, which term refers to a delivery device comprising a chamber or other container portion for holding a liquid to be administered to a subject, wherein the device can deliver the liquid while attached directly to the subject’s skin without the need for a separate support or external reservoir and, typically, permits the subject to be mobile during delivery. The chamber for holding the liquid may be contained in a housing. Typically, an on-body delivery device is affixed to the subject’s skin using an adhesive. The device is affixed sufficiently strongly so that the device is self-supporting. The device may be provided with an adhesive layer, e.g., on the outer surface of the housing, for use to secure the device directly to the skin. The adhesive layer may surround the portion of the device from which a piercing member such as a needle or cannula projects so as to provide a seal around the penetrated skin. In some embodiments an on-body delivery device is available from Sensile Medical AG (Hagendorf, Switzerland). For example, devices known as SenseInfuse, SensePatch, or Senseflex, may be used. In some embodiments an on-body delivery device is available from Enable Injections, Inc. (Cincinnati, OH). In some embodiments the device that comprises a resilient, expandable container portion such as a bladder or balloon to expel the liquid is an on-body delivery device. In some embodiments the device, e.g., an on-body delivery device, is configured such that the piercing member, e.g., needle, is not visible to the user prior to or during use of the device. In some embodiments, the piercing member, e.g., needle, may retract when delivery of the liquid is complete or when the device is removed from the skin. It will be appreciated that a piercing member, e.g., a needle, for use with a delivery device described herein may have any suitable gauge or inner diameter, e.g., such gauge or inner diameters as described elsewhere herein.

In some embodiments, a delivery device comprises a housing into which a vial, cartridge, or syringe containing a liquid (e.g., a liquid comprising a compstatin analog) may be inserted. The liquid is administered upon activation of the device. In some embodiments the liquid is transferred to a chamber of the device prior to administration. In some embodiments a delivery device is reusable, e.g., it can be re-filled or supplied with a new vial, cartridge, or syringe following administration of the contents.

In some embodiments a delivery device is a single use device, i.e., the device is designed to be used to administer a single dose or for use in a single administration session. For example, a device may be designed to be affixed to the skin of a subject, activated to administer a dose, removed, and then recycled or discarded rather than used to administer one or more additional doses.

In some embodiments a delivery device that allows delivery of a liquid into two or more sites may be used. In some embodiments the number of sites is between 1 and 5. In some embodiments the number of sites is greater than 5, e.g., between 6 and 10. Delivery to the two or more sites may be simultaneous or sequential. The device may comprise a pair of syringes, each arranged to be connected to one of the sites and coupled to a body that houses a driving system of the device. Exemplary devices are described in WO2011154928 and U.S. Pat. Application Publication No. 20120143133. In some embodiments a multi-needle infusion set may be used. In some embodiments a multi-needle infusion set comprises a flexible tube that communicates at one end with a chamber (which term is used interchangeably with “reservoir”) containing the liquid (e.g., a syringe) while the other end bifurcates into multiple tubes each having a needle at the end. The neria™ multi infusion sets (Unomedical A/S, Osted, Denmark) are exemplary multi-needle infusion sets.

In some embodiments a delivery device may collect data regarding use of the device. Such data may comprise, for example, the date and time at which the device was used, delivery parameters such as the volume administered, the duration of administration, whether any problems occurred during administration, etc. The data may be stored on a computer-readable medium physically associated with the device and/or may be transmitted to a remote location, e.g., a remote server, where it may be stored, analyzed, or further transmitted for storage or analysis. The device may comprise one or more processors, sensors, software programs, and appropriate connectivity that allow data to be exchanged between the device and other products and systems. Data may be transferred via radio-frequency identification (RFID), bar-code/QRcode scanning, cellular, Bluetooth low energy (BTLE), physical wire, or a combination thereof. The data may be transmitted over any suitable network, e.g., the Internet. The data may be analyzed and/or stored in the Cloud. In some embodiments the device comprises an active or passive RFID tag or chip, hereinafter referred to as an “RFID tag”. The RFID tag may contain data that identifies the device. The RFID tag may be an active tag or chip that signals usage-related information such as activation of the device and/or completion of an administration of a dose. In some embodiments data acquired from a particular device may be made available to one or more entities or individuals, such as health care providers or caregivers of the subject. Such entities or individuals may additionally or alternately be automatically notified of the occurrence or non-occurrence of specified events. For example, if a dose is not administered on a day on which such administration is to take place according to the dosing schedule, or if the device is deployed on a day when administration is not supposed to take place according to the dosing schedule, one or more health care providers or caregivers of the subject may be notified. Once notified, an entity or individual may take appropriate action, such as contacting the subject. In some embodiments a monitoring system automatically attempts to contact the subject, e.g., by phone or text message, if a dose is not administered as scheduled.

In some embodiments a delivery system may comprise a delivery device and a remote control device. The remote control device may, for example, allow programming of the delivery device and/or may be used to activate the delivery device to start delivery of the fluid or to cause the delivery device to cease delivery of the fluid.

In some embodiments, the present disclosure contemplates providing to a subject (e.g., by mail or arranged pickup or other regular mode of delivery) a set of devices as described herein that together provide a supply of active agent (e.g., compstatin analog) sufficient to last for a predetermined period of time (e.g., one week, two weeks, three weeks, four weeks, etc.). In some embodiments, such a set is sent to the patient’s residence on a regular basis (e.g., every week, two weeks, three weeks, four weeks, etc.) with a timing selected such that the patient does not run out. In some embodiments, a composition (e.g., comprising a compstatin analog) may be contained in a container (e.g., a vial) or in any of the herein-mentioned drug delivery devices or packs. In some embodiments the supply is sufficient to last for between 4 and 12 weeks, between 12 and 26 weeks, or more.

Those skilled in the art, reading the present disclosure, will appreciate that, in accordance with standard practice in the field, a container containing a particular volume, as described herein may include an additional volume sufficient to permit the designated particular volume (e.g., unit dose) to be withdrawn from the container for administration.

In particular embodiments, a compstatin analog described herein, e.g., pegcetacoplan, is formulated as a solution in 5% dextrose, as a solution in acetate-buffered mannitol, or as a solution in acetate-buffered sorbitol for subcutaneous administration, e.g., for self-administration subcutaneously. In some embodiments, a compstatin analog described herein, e.g., pegcetacoplan, is provided as a sterile solution of pegcetacoplan, 54 mg/mL, in acetate-buffered sorbitol, supplied in stoppered glass vials. In some embodiments, a compstatin analog described herein, e.g., pegcetacoplan, is provided as a solution that is sterile, isotonic, with a pH of about 5.0, weakly buffered, with an osmolality of between 250 and 350 mOsm/kg. In some embodiments, a compstatin analog described herein, e.g., pegcetacoplan, is administered using a commercially available pump, e.g., a pump described herein, suitable for subcutaneous infusion of about 20 mL.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### VI. Exemplary Treatment Protocol and Efficacy Assessments

In some embodiments, a subject suffering from PNH is treated with APL-2 (“pegcetacoplan”). In some embodiments, the pegcetacoplan is a sterile solution of pegcetacoplan, 54 mg/mL, in acetate-buffered sorbitol. In some embodiments, the pegcetacoplan is supplied in stoppered glass vials. In some embodiments, the pegcetacoplan is administered in a volume of 20 mL. In some embodiments, prior to receiving a first dose of pegcetacoplan, the subject is least 18 years of age, has a primary diagnosis of PNH (e.g., confirmed by high-sensitivity flow cytometry), has a hemoglobin level <10.5 g/dL, has an absolute reticulocyte count >1.0 × Upper Limit of Normal (ULN), has a platelet count of >50,000 /mm³, and/or has an absolute neutrophil count >500 /mm³. In some embodiments, the subject has been vaccinated against Neisseriameningitidis types A, C, W, Y and/or B; and/or against Streptococcuspneumoniae; and/or against Haemophilusinfluenzae Type B (Hib) prior to or within two weeks after receiving a first dose of pegcetacoplan, or the subject is a non-responder to vaccination as evidenced by titers or display titer levels within acceptable local limits. In some embodiments, the subject is not pregnant before or during treatment with pegcetacoplan. In some embodiments, the subject is willing and able to self-administer pegcetacoplan. In some embodiments, pegcetacoplan is administered by a caregiver. In some embodiments, the subject has a body mass index (BMI) <35.0 kg/m². In some embodiments, the subject has a body mass index (BMI) > 35.0 kg/m².

In some embodiments, the subject does not have an active, unresolved bacterial infection prior to receiving a first dose of pegcetacoplan. In some embodiments, the subject is not receiving iron, folic acid, vitamin B12 and/or EPO. In some embodiments, the subject is receiving iron, folic acid, vitamin B12 and/or EPO at a stable dose prior to receiving a first dose of pegcetacoplan. In some embodiments, the subject does not have a hereditary complement deficiency, a history of bone marrow transplantation, and/or a history or presence of hypersensitivity or idiosyncratic reaction to compounds related to pegcetacoplan. In some embodiments, the subject is not breastfeeding at the time of receiving pegcetacoplan.

In some embodiments, the subject does not have a history or family history of Long QT Syndrome or torsade de pointes, unexplained syncope, syncope from an uncorrected cardiac etiology, and/or family history of sudden death. In some embodiments, the subject does not have myocardial infarction, CABG, coronary or cerebral artery stenting and/or angioplasty, stroke, cardiac surgery, and/or hospitalization for congestive heart failure within 3 months or > Class 2 Angina Pectoris or NYHA Heart Failure Class >2. In some embodiments, the subject does not have QTcF >470 ms or PR >280 ms. In some embodiments, the subject does not have Mobitz II 2nd degree AV Block, 2:1 AV Block, High Grade AV Block, or Complete Heart Block unless the subject has an implanted pacemaker or implantable cardiac defibrillator (ICD) with backup pacing capabilities. In some embodiments, the subject is not receiving Class 1 or Class 3 antiarrhythmic agents, or arsenic, methadone, ondansetron or pentamidine. In some embodiments, the subject is not receiving a QTc-prolonging drug at a stable dose prior to receiving a first dose of pegcetacoplan. In some embodiments, the subject is not receiving prophylactic ciprofloxacin, erythromycin and/or azithromycin prior to a first dose of pegcetacoplan. In some embodiments, the subject has an ECG after one week of prophylactic antibiotics with QTcF <470 ms.

In some embodiments, the subject has received < 4 PRBC transfusions within the 12 months prior to treatment with pegcetacoplan. In some embodiments, the subject has received at least 4 PRBC transfusions within the 12 months prior to treatment with pegcetacoplan.

In some embodiments, the subject’s platelet count prior to receiving a first dose of pegcetacoplan is <100,000 /mm³. In some embodiments, the subject’s platelet count prior to receiving a first dose of pegcetacoplan is ≥100,000 /mm³.

In some embodiments, the subject is on treatment with eculizumab. In some embodiments, the subject’s current dose of eculizumab has been stable for at least 3 months prior to receiving a first dose of pegcetacoplan.

In some embodiments, the subject is subcutaneously administered pegcetacoplan in a 1080 mg dose twice weekly. In some embodiments, the twice weekly doses are administered on days 1 and 4 of a given treatment week. In some embodiments, the subject is subcutaneously administered pegcetacoplan in a 1080 mg dose every three days.

In some embodiments, the subject is subcutaneously administered pegcetacoplan in a 1080 mg dose twice weekly and is administered eculizumab at the current dose (e.g., the amount and frequency administered prior to treatment with pegcetacoplan) for four weeks, and after four weeks the treatment with eculizumab is discontinued and the subject continues to be subcutaneously administered pegcetacoplan in a 1080 mg dose twice weekly. In some embodiments, the subject is subcutaneously administered pegcetacoplan in a 1080 mg dose every three days and is administered eculizumab at the current dose (e.g., the amount and frequency administered prior to treatment with pegcetacoplan) for four weeks, and after four weeks the treatment with eculizumab is discontinued and the subject continues to be subcutaneously administered pegcetacoplan in a 1080 mg dose every three days.

In some embodiments, the subject is subcutaneously administered pegcetacoplan in a 1080 mg dose at a frequency determined according to the subject’s LDH level and is administered eculizumab at the current dose (e.g., the dose administered prior to treatment with pegcetacoplan) for four weeks, and after four weeks the treatment with eculizumab is discontinued and the subject continues to be subcutaneously administered pegcetacoplan in a 1080 mg dose at a frequency determined according to the subject’s LDH level. In some embodiments, if the subject’s LDH level is less than or equal to 2× the upper limit of normal, the subject is subcutaneously administered pegcetacoplan in a 1080 mg dose twice weekly. In some embodiments, if the subject’s LDH level is greater than 2× the upper limit of normal, the subject is subcutaneously administered pegcetacoplan in a 1080 mg dose every three days. In some embodiments, the subject’s dosing frequency of pegcetacoplan is adjusted based on the subject’s LDH level. In some embodiments, the upper limit of normal for LDH level in the subject is about 225 U/L.

In some embodiments, the subject is subcutaneously administered pegcetacoplan in a 1080 mg dose at a frequency determined according to the subject’s LDH level. In some embodiments, if the subject’s LDH level is less than or equal to 2× the upper limit of normal, the subject is subcutaneously administered pegcetacoplan in a 1080 mg dose twice weekly. In some embodiments, if the subject’s LDH level is greater than 2× the upper limit of normal, the subject is subcutaneously administered pegcetacoplan in a 1080 mg dose every three days. In some embodiments, the subject’s dosing frequency of pegcetacoplan is adjusted based on the subject’s LDH level. In some embodiments, the upper limit of normal for LDH level in the subject is about 225 U/L.

In some embodiments, in the event of a pegcetacoplan dose increase from twice weekly to every three days, LDH is monitored bi-weekly for at least four weeks to assess the impact of the dose adjustment on LDH levels.

In some embodiments, the subject is treated for at least 16 weeks. In some embodiments, the subject is treated at least 48 weeks. In some embodiments, the subject is treated for 52 weeks.

In some embodiments, the subject’s baseline health and/or response to pegcetacoplan is evaluated using hematology. In some embodiments, the evaluation includes assessing one or more of hemoglobin, hematocrit, RBC count, platelet count, white blood cell count with differential, and reticulocytes. In some embodiments, the subject’s baseline health and/or response to pegcetacoplan is evaluated using coagulation. In some embodiments, the evaluation includes assessing one or more of prothrombin time (PT), fibrinogen, activated partial thromboplastin time (aPTT), and D-Dimer. In some embodiments, the subject’s baseline health and/or response to pegcetacoplan is evaluated using serum chemistry. In some embodiments, the evaluation includes assessing one or more of blood urea nitrogen (BUN), creatinine, estimated creatinine clearance (using Cockcroft-Gault formula), bilirubin (total and direct), albumin, alkaline phosphatase (ALP), lactate dehydrogenase (LDH), haptoglobin, gamma-glutamyl transpeptidase (GGT), lactate dehydrogenase ioenzymes, vitamin B12, creatine kinase (CK), aspartate aminotransferase (AST), alanine aminotransferase (ALT), uric acid, glucose, sodium, potassium, chloride, ferritin, erythropoietin, folate, calcium, and phosphate. In some embodiments, the subject’s baseline health and/or response to pegcetacoplan is evaluated using urinalysis. In some embodiments, the evaluation includes assessing one or more of pH, specific gravity, protein, glucose, ketones, bilirubin, blood, nitrite, urobilinogen, and leukocyte esterase.

In some embodiments, the subject is evaluated for hemoglobin level. In some embodiments, the evaluation involves assessing a change in hemoglobin level from baseline to a period after receiving a first dose of pegcetacoplan. In some embodiments, hemoglobin level is assessed at 16 weeks after the subject’s first dose of pegcetacoplan. In some embodiments, hemoglobin level is assessed at 20, 24, 28, 32, 36, 40, 44, 48, or 52 weeks after the subject’s first dose of pegcetacoplan. In some embodiments, the subject’s hemoglobin level increases compared to baseline. In some embodiments, the subject’s hemoglobin level increases compared to baseline by at least 1 g/dL. In some embodiments, the subject’s hemoglobin level increases compared to baseline by at least 2 g/dL. In some embodiments, the subject’s hemoglobin level increases compared to baseline by at least 3 g/dL. In some embodiments, the subject’s hemoglobin level increases compared to baseline by at least 4 g/dL. In some embodiments, the subject’s hemoglobin level increases compared to baseline by about 2.4 g/dL. In some embodiments, the subject’s hemoglobin level increases to about 11 to 12 g/dL. In some embodiments, the subject’s increase in hemoglobin level is sustained for at least 16 weeks after receiving a first dose of pegcetacoplan. In some embodiments, the subject’s increase in hemoglobin level is sustained for at least 20, 24, 28, 32, 36, 40, 44, 48, or 52 weeks after receiving a first dose of pegcetacoplan.

In some embodiments, the subject is evaluated for transfusion avoidance. In some embodiments, the evaluation includes assessing a change in transfusion avoidance from baseline to a period after receiving a first dose of pegcetacoplan. In some embodiments, transfusion avoidance is assessed at 16 weeks after the subject’s first dose of pegcetacoplan. In some embodiments, transfusion avoidance is assessed at 20, 24, 28, 32, 36, 40, 44, 48, or 52 weeks after the subject’s first dose of pegcetacoplan. In some embodiments, the subject’s transfusion avoidance improves compared to baseline, e.g., in some embodiments, the subject requires fewer transfusions while receiving pegcetacoplan than prior to receiving pegcetacoplan. In some embodiments, the subject requires at least 1, 2, 3, 4, 5, or 6 fewer transfusions compared to baseline. In some embodiments, the subject requires fewer that 3, 2, or 1 transfusions over a period of at least 4 weeks, at least 8 weeks, at least 12 weeks, at least 16 weeks, at least 20 weeks, or at least 24 weeks following the subject’s first dose of pegcetacoplan.

In some embodiments, the subject is evaluated for reticulocyte level (i.e., absolute reticulocyte count). In some embodiments, the evaluation includes assessing a change in reticulocyte level from baseline to a period after receiving a first dose of pegcetacoplan. In some embodiments, reticulocyte level is assessed at 16 weeks after the subject’s first dose of pegcetacoplan. In some embodiments, reticulocyte level is assessed at 20, 24, 28, 32, 36, 40, 44, 48, or 52 weeks after the subject’s first dose of pegcetacoplan. In some embodiments, the subject’s reticulocyte level decreases compared to baseline. In some embodiments, the subject’s reticulocyte level decreases compared to baseline by at least about 135 × 10⁹ /L, e.g., in some embodiments by about 135 × 10⁹ /L. In some embodiments, the subject’s reticulocyte level decreases to about 60-85 × 10⁹ /L. In some embodiments, the subject’s reticulocyte level decreases to about 70-80 × 10⁹ /L. In some embodiments, the subject’s reticulocyte level decreases to about 77 × 10⁹ /L. In some embodiments, the subject’s decrease in reticulocyte level is sustained for at least 16 weeks after receiving a first dose of pegcetacoplan. In some embodiments, the subject’s decrease in reticulocyte level is sustained for at least 20, 24, 28, 32, 36, 40, 44, 48, or 52 weeks after receiving a first dose of pegcetacoplan.

In some embodiments, the subject is evaluated for LDH level. In some embodiments, the evaluation includes assessing a change in LDH level from baseline to a period after receiving a first dose of pegcetacoplan. In some embodiments, LDH level is assessed at 16 weeks after the subject’s first dose of pegcetacoplan. In some embodiments, LDH level is assessed at 20, 24, 28, 32, 36, 40, 44, 48, or 52 weeks after the subject’s first dose of pegcetacoplan. In some embodiments, the subject’s LDH level decreases compared to baseline. In some embodiments, the subject’s LDH level decreases compared to baseline by at least about 15 U/L, e.g., in some embodiments by about 15 U/L. In some embodiments, the subject’s reticulocyte level decreases to about 160-230 U/L. In some embodiments, the subject’s reticulocyte level decreases to about 180-195 U/L, e.g., in some embodiments to about 189 U/L or about 190 U/L. In some embodiments, the subject’s decrease in reticulocyte level is sustained for at least 16 weeks after receiving a first dose of pegcetacoplan. In some embodiments, the subject’s decrease in reticulocyte level is sustained for at least 20, 24, 28, 32, 36, 40, 44, 48, or 52 weeks after receiving a first dose of pegcetacoplan.

In some embodiments, the subject is evaluated for fatigue level. In some embodiments, the evaluation includes assessing a change in fatigue level from baseline to a period after receiving a first dose of pegcetacoplan. In some embodiments, fatigue level is assessed at 16 weeks after the subject’s first dose of pegcetacoplan. In some embodiments, fatigue level is assessed at 20, 24, 28, 32, 36, 40, 44, 48, or 52 weeks after the subject’s first dose of pegcetacoplan. In some embodiments, the subject’s amount of fatigue decreases compared to baseline. In some embodiments, fatigue level is assessed according to the Functional Assessment of Chronic Illness Therapy (FACIT)-fatigue scale score, e.g., in some embodiments, fatigue level is assessed according the FACIT-fatigue scale score Version 4. In some embodiments, the subject’s FACIT-fatigue scale score improves (increases) compared to baseline. In some embodiments, the subject’s FACIT-fatigue scale score increases by at least 3 points compared to baseline. In some embodiments, the subject’s FACIT-fatigue scale score increases by at least about 5-20 points compared to baseline. In some embodiments, the subject’s FACIT-fatigue scale score increases by about 7.5-11 points compared to baseline, e.g., in some embodiments the subject’s score increases by about 9 points compared to baseline. In some embodiments, the subject’s FACIT-fatigue scale score increases to about 32, 34, 36, 38, 40, 42, 44, 46, or 48. In some embodiments, the subject’s FACIT-fatigue scale score increases to about 40 to 44. In some embodiments, the subject’s increase in FACIT-fatigue scale score is sustained for at least 16 weeks after receiving a first dose of pegcetacoplan. In some embodiments, the subject’s increase in FACIT-fatigue scale score is sustained for at least 20, 24, 28, 32, 36, 40, 44, 48, or 52 weeks after receiving a first dose of pegcetacoplan.

In some embodiments, the subject is evaluated for hemoglobin response (an increase of at least ≥1 g/dL in hemoglobin) in the absence of a transfusion. In some embodiments, the subject is evaluated for reticulocyte normalization (reticulocyte count below the upper limit of the normal range) in the absence of a transfusion. In some embodiments, the subject is evaluated for hemoglobin normalization (hemoglobin level above the lower limit of the normal range) in the absence of a transfusion.

In some embodiments, the subject is evaluated for changes compared to baseline levels of bilirubin, haptoglobin, Linear Analog Scale Assessment (LASA) score, European Organisation for Research and Treatment of Cancer (EORTC) QLQ-C30 score, and/or number of PRBC units transfused. In some embodiments, the change from baseline is assessed at 16 weeks after the subject’s first dose of pegcetacoplan. In some embodiments, the change from baseline is assessed at 20, 24, 28, 32, 36, 40, 44, 48, or 52 weeks after the subject’s first dose of pegcetacoplan. In some embodiments, the subject’s change from baseline is sustained for at least 16 weeks after receiving a first dose of pegcetacoplan. In some embodiments, the subject’s change from baseline is sustained for at least 20, 24, 28, 32, 36, 40, 44, 48, or 52 weeks after receiving a first dose of pegcetacoplan.

In some embodiments, pharmacokinetics and pharmacodynamics are assessed. In some embodiments, the evaluation includes assessing the subject’s change from baseline in percentage of PNH Type II + III RBCs and/or the subject’s change from baseline in percentage of PNH Type II + III RBCs opsonized with C3. In some embodiments, the change from baseline is assessed at 16 weeks after the subject’s first dose of pegcetacoplan. In some embodiments, the change from baseline is assessed at 20, 24, 28, 32, 36, 40, 44, 48, or 52 weeks after the subject’s first dose of pegcetacoplan. In some embodiments, the subject’s change from baseline is sustained for at least 16 weeks after receiving a first dose of pegcetacoplan. In some embodiments, the subject’s change from baseline is sustained for at least 20, 24, 28, 32, 36, 40, 44, 48, or 52 weeks after receiving a first dose of pegcetacoplan.

In some embodiments, incidence and severity of treatment-emergent adverse events (TEAEs), incidence of thromboembolic events, changes from baseline in laboratory parameters, and/or changes from baseline in electrocardiogram (ECG) parameters are assessed. In some embodiments, these incidences, severities, and/or changes are assessed at 16 weeks after the subject’s first dose of pegcetacoplan. In some embodiments, they are assessed at 20, 24, 28, 32, 36, 40, 44, 48, or 52 weeks after the subject’s first dose of pegcetacoplan.

### Example 1 - PEGASUS (16-Week Study Results)

This Example describes 16-week results from a Phase 3 randomized, multi-center, open-label, active-comparator controlled study to evaluate the efficacy and safety of APL-2 (pegcetacoplan, also referred to as “Study Drug” in this Example) in patients with PNH.

### Methods

### Patient Selection

The PEGASUS trial protocol consisted of an 8-week screening period, a 52-week treatment period, and a 12-week follow up period. A schematic of the PEGASUS trial as conducted is shown in FIG. 1B.

At screening, male and female subjects were required to fulfill all of the following inclusion criteria to be eligible for participation in the study:
At least 18 years of age.
Primary diagnosis of PNH confirmed by high-sensitivity flow cytometry.
On treatment with eculizumab. Dose of eculizumab must have been stable for at least 3 months prior to the Screening Visit.
Hemoglobin level <10.5 g/dL at the Screening Visit.
Absolute reticulocyte count >1.0 × Upper Limit of Normal (ULN) at the Screening Visit.
Platelet count of >50,000 /mm³ at the Screening Visit.
Absolute neutrophil count >500 /mm³ at the Screening Visit.
Vaccination against Neisseriameningitidis types A, C, W, Y and B, Streptococcuspneumoniae and Haemophilusinfluenzae Type B (Hib) either within 2 years prior to Day 1 dosing, or within 14 days after starting treatment with study drug (unless documented evidence existed that subjects were non-responders to vaccination as evidenced by titers or display titer levels within acceptable local limits).
Women of child-bearing potential (WOCBP) had a negative pregnancy test at the Screening and Day -28 Visit (Run-in Period) and agreed to use protocol defined methods of contraception for the duration of the study and 90 days after their last dose of study drug.
Males agreed to use protocol defined methods of contraception and agreed to refrain from donating sperm for the duration of the study and 90 days after their last dose of study drug.
Approved methods of contraception included: oral contraceptives, intrauterine device, medically acceptable barrier methods (diaphragm or condom), implantable or injectable contraceptives (like Depo Provera) or removable birth control device (like NuvaRing or Ortho Evra patches); and/or surgical sterilization (at least 6 months before dosing).
Willing and able to give informed consent.
Willing and able to self-administer study drug (administration by caregiver was allowed).
Had a body mass index (BMI) <35.0 kg/m².

Subjects were excluded from the study if there was evidence of any of the following criteria at screening and confirmed at the Day -28 Visit, as appropriate:
Active bacterial infection that was not resolved within 1 week of Day -28 (first dose of study drug).
Receiving iron, folic acid, vitamin B12 and EPO, unless the dose was stable, in the 4 weeks prior to Screening.
Hereditary complement deficiency.
History of bone marrow transplantation.
History or presence of hypersensitivity or idiosyncratic reaction to compounds related to the investigational product of SC administration.
Participation in any other investigational drug trial or exposure to other investigational agent within 30 days or 5 half-lives (whichever was longer).
Currently breast-feeding women.
Inability to cooperate or any condition that, in the opinion of the investigator, could increase the subject’s risk of participating in the study or confound the outcome of the study.
History or family history of Long QT Syndrome or torsade de pointes, unexplained syncope, syncope from an uncorrected cardiac etiology, or family history of sudden death.
Myocardial infarction, CABG, coronary or cerebral artery stenting and/or angioplasty, stroke, cardiac surgery, or hospitalization for congestive heart failure within 3 months or > Class 2 Angina Pectoris or NYHA Heart Failure Class >2.
QTcF >470 ms, PR >280 ms.
Mobitz II 2nd degree AV Block, 2:1 AV Block, High Grade AV Block, or Complete Heart Block unless the patient had an implanted pacemaker or implantable cardiac defibrillator (ICD) with backup pacing capabilities.
Receiving Class 1 or Class 3 antiarrhythmic agents, or arsenic, methadone, ondansetron or pentamidine at screening.
Receiving a QTc-prolonging drug at a stable dose for less than 3 weeks prior to dosing.
Receiving prophylactic ciprofloxacin, erythromycin or azithromycin for less than one week prior to the first dose of study medication (must have a repeat screening ECG after one week of prophylactic antibiotics with QTcF <470 ms).

### Vaccinations

Vaccination, or evidence of vaccination, was required for N.meningitidis, H. influenzae Type B (Hib), and S. pneumoniae. If the subject’s first documented N. meningitidis vaccine(s) were administered during the run-in period (Day -14), a booster (for both vaccinations) was to be administered after 2 months. If not previously documented, subjects were also to be vaccinated against H. influenzae Type B (Hib). Vaccination was mandatory unless documented evidence existed that subjects were nonresponders to vaccination as evidenced by titers or display titer levels within acceptable local limits.

S.pneumoniae vaccination requirement scenarios were as follows (unless documented evidence existed that subjects were nonresponders to vaccination as evidenced by titers or display titer levels within acceptable local limits):

If, within 2 years prior to initiating treatment with pegcetacoplan, the subject had documented S. pneumoniae vaccination with both the PCV13 vaccine and the PPSV23 vaccine, no additional S. pneumoniae vaccination was required for study entry.
If, within 2 years prior to initiating treatment with pegcetacoplan, the subject had no documented S. pneumoniae vaccination with either the PCV13 vaccine or the PPSV23 vaccine, then the subject must have received the PCV13 vaccine within 2 weeks of Day 1, followed by the PPSV23 vaccine after at least 8 weeks, as indicated in the protocol.
If, within 2 years prior to initiating treatment with pegcetacoplan, the subject had documented S. pneumoniae vaccination with the PCV13 vaccine only, then the subject must have received the PPSV23 vaccine within 2 weeks prior to Day 1, followed by a PPSV23 booster vaccine at least 8 weeks later.
If, within 2 years prior to initiating treatment with pegcetacoplan, the subject had documented S. pneumoniae vaccination with the PPSV23 vaccine only, then the subject must have received the PPSV23 booster vaccine within 2 weeks prior to Day 1.

### Study Design

Subjects were randomized to receive either Study Drug or eculizumab. The treatment period consisted of three parts: a 4-week run-in period, a 16-week Randomized Controlled Period and a 32-week open-label Study Drug only period.

During the 4-week run-in period (Week -4 to Day -1) all subjects self-administered twice-weekly subcutaneous doses of Study Drug (1,080 mg), in addition to receiving the subjects’ current dose of eculizumab treatment, which continued as prescribed regardless of Study Visit scheduling or the Study Drug administration schedule (i.e., it was not required that eculizumab dosing aligned with Study Drug dosing or Study Visits). On Day 1, subjects received their doses of Study Drug and may have received eculizumab depending on their dosing schedules. Subjects were then randomized to either Group 1 (monotherapy Study Drug) or Group 2 (monotherapy eculizumab). Subjects in Group 1 received Study Drug, and subjects in Group 2 received eculizumab for the remainder of the 16-week Randomized Controlled Period. During the Randomized Controlled Period, subjects returned to the clinical site at Weeks 1, 2, 4, 6, 8, 12 and 16 for efficacy and safety assessments.

The randomization was stratified by the following values:
Number of PRBC transfusions within the 12 months prior to Day -28 (<4; ≥4) (i.e., number of transfusion events regardless of PRBC units transfused)
Platelet count at screening (<100,000; ≥100,000)

The sample size included approximately 50% of the subjects in each strata (PRBC transfusions <4, PRBC transfusions ≥4). Enrollment of subjects with <4 transfusions was limited to ≤50%.

Day 1 to Week 16 was defined as the Randomized Controlled Period, over which endpoints were assessed. After completion of the Randomized Controlled Period (the end of Week 16), all subjects continue into a 32-week Open-Label Study Drug Period in which all subjects receive twice-weekly doses of Study Drug (1,080 mg). During this period, subjects return to the clinical site on Weeks 17, 18, 20, 22 and 24 and every 4 weeks, thereafter, until Week 48 for efficacy and safety assessments. Those subjects who received eculizumab in the Randomized Controlled Period receive Study Drug in addition to eculizumab for 4 weeks (Weeks 17-20).

After completion of the 52-week treatment period (Week 48), subjects are offered entry into an open label extension study. Subjects who do not enter the open label extension study exit the study and return to the site for 2 additional safety visits 6 weeks apart. The end of the trial is defined as when the last subject either completes their Week 48 visit and enroll in the long-term safety extension (LTSE) study, or, for subjects who elect not to enter the LTSE study, when the last subject completes their exit visit at Week 60.

Subjects who withdraw from treatment prior to the Week 48 visit continue their participation in the study and return to the study site for their scheduled study procedures, with the exception of Study Drug administration. Subjects who withdraw from the study prior to Week 48 and are being treated solely with Study Drug receive at least one dose of eculizumab before discontinuing Study Drug.

The length of participation in the study for each subject is a maximum of approximately 72 weeks, including an 8-week screening period, 52-week treatment period and 12-week follow-up period. Those who enter the open label extension study do not require the 12-week follow-up period.

### Clinical Laboratory Tests

The clinical laboratory tests included (but were not limited to) the following:
Hematology: Hb, Hematocrit, RBC count, Platelet count, WBC count with differential, Reticulocytes.
Coagulation: Prothrombin time (PT), Fibrinogen, Activated partial thromboplastin time (aPTT), D-Dimer.
Serum Chemistry: Blood urea nitrogen (BUN), Creatinine, Estimated creatinine clearance (using Cockcroft-Gault formula) -screening only, Bilirubin (total and direct), Albumin, Alkaline phosphatase (ALP), Lactate dehydrogenase (LDH), Haptoglobin, Gamma-glutamyl transpeptidase (GGT), Lactate Dehydrogenase Isoenzymes, Vitamin B12, Creatine kinase (CK), Aspartate aminotransferase (AST), Alanine Aminotransferase (ALT), Uric acid, Glucose, Sodium, Potassium, Chloride, Ferritin, Erythropoietin, Folate, Calcium, Phosphate.
Urinalysis: pH, Specific gravity, Protein, Glucose, Ketones, Bilirubin, Blood, Nitrite, Urobilinogen, Leukocyte esterase.

### Study Treatments

The Study Drug was pegcetacoplan (also referred to as “APL-2”) (see FIG. 1A), which was provided as a sterile solution of pegcetacoplan, 54 mg/mL, in acetate-buffered sorbitol, supplied in stoppered glass vials.

Starting on Day -28 (Visit 2), subjects received self-administered twice-weekly subcutaneous (SC) doses of 1,080 mg pegcetacoplan in addition to their current dose of eculizumab until Day 1. Subjects maintained their eculizumab dose and administration schedule as prescribed. On Day 1, subjects received their dose of pegcetacoplan and may have received eculizumab depending on their dosing schedule. Subjects were then randomized to either Group 1 (monotherapy pegcetacoplan) or Group 2 (monotherapy eculizumab).

Subjects in Group 1 stop their eculizumab treatment and continue to receive pegcetacoplan (1,080 mg twice a week) on Day 1 and Day 4 of each treatment week until the end of Week 48.

Subjects in Group 2 continue to receive their pre-screening stable dose of eculizumab until the end of Week 20. Following their Week 16 visit subjects receive pegcetacoplan (1,080 mg twice a week) on Day 1 and Day 4 of the treatment week until the end of Week 48.

Following commencement of monotherapy with pegcetacoplan, at Day 1 (randomization), lactate dehydrogenase (LDH) was monitored as part of the scheduled assessments at the planned clinic visits. For subjects receiving pegcetacoplan monotherapy, if LDH was >2 × ULN, a pegcetacoplan dose increase to 1,080 mg every third day was initiated. In the event of a dose increase, LDH was monitored bi-weekly (unscheduled assessments if applicable) for at least four weeks to assess the impact of the dose adjustment on LDH levels. Following commencement of monotherapy with pegcetacoplan at Week 21, lactate dehydrogenase (LDH) is monitored as part of the scheduled assessments at the planned clinic visits. For subjects receiving pegcetacoplan monotherapy, if LDH is >2 × ULN, a pegcetacoplan dose increase to 1,080 mg every third day is initiated. In the event of a dose increase, LDH is monitored bi-weekly (unscheduled assessments if applicable) for at least four weeks to assess the impact of the dose adjustment on LDH levels.

### Measures of Clinical Efficacy

The primary endpoint in the study was an increase in hemoglobin level from baseline to Week 16 (excluding data before the Randomized Controlled Period). Key secondary endpoints included transfusion avoidance, reduction in reticulocyte count, reduction in LDH level, and changes in Functional Assessment of Chronic Illness Therapy (FACIT)-fatigue scale score, Version 4. Additional secondary endpoints included hemoglobin response in the absence of transfusions (with hemoglobin response defined as an increase of at least ≥1 g/dL in hemoglobin from Baseline at Week 16, excluding data before the Randomized Controlled Period); reticulocyte normalization in the absence of transfusions (with reticulocyte normalization defined as the reticulocyte count being below the upper limit of the normal range at Week 16); hemoglobin normalization in the absence of transfusions (with hemoglobin normalization defined as the hemoglobin level being above the lower limit of the normal range at Week 16); change from Baseline to Week 16, excluding data before the Randomized Controlled Period, in indirect bilirubin level; change from Baseline to Week 16, excluding data before the Randomized Controlled Period, in haptoglobin level; change from Baseline to Week 16, excluding data before the Randomized Controlled Period, in Linear Analog Scale Assessment (LASA) scores; change from Baseline to Week 16, excluding data before the Randomized Controlled Period, in European Organisation for Research and Treatment of Cancer (EORTC) QLQ-C30 scores; number of PRBC units transfused during the Randomized Controlled Period [Day 1 to Week 16 and Week 4 to Week 16]; change from Baseline and change from Week 17 to Week 48 in hemoglobin level; change from Baseline and change from Week 17 to Week 48 in reticulocyte count; change from Baseline and change from Week 17 to Week 48 in lactate dehydrogenase (LDH) level; change from Baseline and change from Week 17 to Week 48 in FACIT-fatigue scale score; change from Baseline and change from Week 17 to Week 48 in LASA scores; change from Baseline and change from Week 17 to Week 48 in QLQ-C30 scores; and number of PRBC units transfused during the Open-Label Study Drug Period. Hierarchical significance testing for secondary efficacy endpoints was gated on the success of the primary efficacy endpoint. Post hoc analyses included hemoglobin stabilization (defined as avoidance of a >1 g/dL decrease from baseline) in the absence of transfusions and hematologic response to treatment.

Pharmacokinetics and pharmacodynamics were also assessed, including change from baseline in percentage of PNH Type II + III RBCs at week 16, and change from baseline in percentage of PNH Type II + III RBCs opsonized with C3 at week 16. Incidence and severity of treatment-emergent adverse events (TEAEs), incidence of thromboembolic events, changes from baseline in laboratory parameters, and changes from baseline in electrocardiogram (ECG) parameters were also assessed.

### Statistical Analysis

The primary endpoint was assessed on ITT set (included all subjects who were randomized), and change from Baseline to Week 16 in Hb level was measured. Key secondary endpoints were tested in hierarchical manner after statistical significance was reached for the primary endpoint, and were performed on ITT set. If one hypothesis was tested as not significant, all subsequent tests were not assessed. Estimates were computed for key secondary endpoints regardless of whether a hypothesis was tested not significant preventing assessment of further tests. Safety Analysis was conducted in the safety set (which included all subjects who were randomized and received at least 1 dose of monotherapy Study Drug).

The between-treatment group comparison for the primary efficacy endpoint was performed using a mixed effect model for repeated measures (MMRM) (Mallinckrodt, DID 42:30 (2008)). The model included fixed categorical effects for treatment group, study visit, stratification variables (based on transfusion history and platelet count) and the study visit-bytreatment group interaction, as well as the continuous, fixed covariate of baseline Hb level. The difference between APL-2 and eculizumab mean Hb changes from baseline at Week 16 were calculated along with its 2-sided 95% CI and associated p-value from the MMRM model.

The analyses of key secondary efficacy endpoints were based on non-inferiority tests. Noninferiority was concluded if the appropriate limit of the 95% 2-sided confidence interval indicated APL-2 was not inferior to eculizumab by the defined non-inferiority margin for each key secondary efficacy endpoints, as discussed below.

For transfusion avoidance, the number and percentage of subjects in the following categories were presented by treatment group:
no transfusions over the Randomized controlled period (Day 1 to Week 16)
received a transfusion during the Randomized controlled period
withdrew from the study without having had a transfusion during the Randomized controlled period

Subjects who did not have a transfusion but withdrew before Week 16 were considered as having a transfusion in the analysis of transfusion avoidance. The number and percentage of subjects with transfusion avoidance were tabulated by treatment group and compared between treatment groups using a stratified Cochran-Mantel Haenszel (CMH) chi-square test. The treatment difference in percentages and 95% confidence interval for the difference were presented using the stratified method (Miettinen, Statistics in Medicine 4:213-226 (1985)). If the lower bound of the 95% CI for the difference between APL-2 and eculizumab treatment groups was greater than the non-inferiority margin of -20%, then APL-2 was considered non-inferior to eculizumab.

The change from baseline at Week 16 in reticulocyte count, LDH level and FACIT-fatigue scale score were analyzed using the same methods described for the primary analysis of the primary efficacy endpoint except using their own baseline as a covariate, using the ITT and mITT sets. For reticulocyte count, if the upper bound of the 95% CI for the treatment difference was less than the non-inferiority margin of 10, then APL-2 was considered non-inferior to eculizumab. For LDH, if the upper bound of the 95% CI for the treatment difference was less than the non-inferiority margin of 20, then APL-2 was considered non-inferior to eculizumab. For FACIT-fatigue score, if the lower bound of the 95% CI for the treatment difference was greater than the non-inferiority margin of -3 then APL-2 was considered non-inferior to eculizumab.

Any subject who received a transfusion during the Randomized controlled period or withdrew from the study was considered to have experienced an intercurrent event, and all subsequent values were set to missing (While on-Treatment Strategy) for the following parameters (hemoglobin value, absolute reticulocyte count, LDH level, bilirubin level, haptoglobin level, FACIT-fatigue scale score, LASA scores, QLQ-C30 scores). For any subject who discontinued study treatment, any values collected after discontinuation continued to be used in analyses (Treatment Policy).

Subjects were divided into various sets. The Run-in Set included all subjects who received at least one dose of APL-2. The Intent-to-Treat (ITT) Set included all subjects who were randomized. The analyses using this set was based upon the randomized treatment group allocated. The Safety Set included all subjects who were randomized and received at least 1 dose of monotherapy Study Drug. This set was used for safety analyses. The analyses using this set were based upon the actual treatment received. The Modified ITT (mITT) Set included all subjects in the ITT set who received at least one dose of monotherapy beyond their Week 4 after randomization in the Randomized Controlled Period. The analyses using this set were based upon the randomized treatment group allocated. The Per-protocol (PP) Set included all subjects in the ITT set who did not violate any inclusion or exclusion criteria and/or deviated from the protocol in a way that could influence their efficacy assessment. Subjects were required to receive their randomized treatment to be included in the set and so analyses using this set were by default based upon the actual treatment group allocated. The Completer Set consisted of all subjects in the ITT set who completed the Week 16 efficacy assessment for the study. The analyses using this set were based upon the randomized treatment group allocated. The numbers of subjects in the various sets are provided in the following Table:

TABLE 2

Analysis Sets

| Analysis Population | Statistics | APL-2 Group (N=41) | Eculizumab Group (N=39) | Total (N=80) |
|---|---|---|---|---|
| Intent-to-treat Set | n (%) | 41 (100) | 39 (100) | 80 (100) |
| Safety Set | n (%) | 41 (100) | 39 (100) | 80 (100) |
| Modified ITT Set (mlTT) | n (%) | 41 (100) | 39(100) | 80 (100) |
| Per-protocol Set (PP) | n (%) | 36 (87.8) | 35 (89.7) | 71 (88.8) |
| Completer Set | n (%) | 37 (90.2) | 38 (97.4) | 75 (93.8) |

Additionally, the primary endpoint of hemoglobin change from baseline (before first dose of pegcetacoplan) to week 16 and secondary endpoints (transfusion avoidance, change from baseline at week 16 in absolute reticulocyte count and lactate dehydrogenase) were analyzed by subgroups based on number of packed red blood cell transfusions (<4 vs ≥4) within the 12 months prior to Day -28 and platelet count at screening (<100,000 /mm³ vs >100,000 /mm³).

### Results

### Subject Disposition

A total of 80 PNH patients participated in the study, as summarized in the following Tables:

TABLE 3

Subject Disposition

| | APL-2 Group (N=41) | Eculizumab Group (N=39) | Total (N=80) |
|---|---|---|---|
| | n (%) | n (%) | n (%) |
| Received at Least One Dose of Study Drug | 41 (100) | 39 (100) | 80 (100) |
| Completed week 16 Treatment | 38 (92.7) | 39 (100) | 77 (96.3) |
| Withdrawn from Study Treatment | 3 (7.3) | 0 | 3 (3.8) |
| Primary Reason for Withdrawl from Study Treatment | | | |
| Adverse Events* | 3 (7.3) | 0 | 3 (3.8) |

*Adverse events leading to withdrawal were all reported as hemolysis

TABLE 4

Subject Demographics

| Characteristic | Pegcetacoplan Group (n=41) | Eculizumab Group (n=39) |
|---|---|---|
| Age - yr | | |
| Mean (range) | 50.2 (19, 81) | 47.3 (23, 78) |
| >65 yr - no. (%) | 10 (24.4) | 7 (17.9) |
| Female sex - no. (%) | 27 (65.9) | 22 (56.4) |
| Race - no. (%) | | |
| Asian | 5 (12.2) | 7 (17.9) |
| Black/African American | 2 (4.9) | 0 |
| White | 24 (58.5) | 25 (64.1) |
| Other | 0 | 1 (2.6) |
| Not reported | 10 (24.4) | 6 (15.4) |
| Body mass index — mean (SD) kg/m² | 26.7 (4.3) | 25.9 (4.3) |

TABLE 5

Baseline Characteristics

| Characteristic | Pegcetacoplan Group (n=41) | Eculizumab Group (n=39) |
|---|---|---|
| Time since PNH diagnosis - median (range) yr | 6.0(1, 31) | 9.7 (1, 38) |
| Duration of prior treatment with eculizumab - median (range) days | 1618 (155,6231) | 1254 (118, 5047) |
| Eculizumab dose at screening - no. (%) | | |
| 900 mg every 2 wk | 26 (63.4) | 30 (76.9) |
| 1200 mg every 2 wk^{a} | 13 (31.7) | 9 (23.1) |
| 1500 mg every 2 wk | 2 (4.9) | 0 |
| Platelets - mean (SD) ×10⁹ /I | 166.6 (98.3) | 146.9 (68.8) |
| ≥4 transfusions in previous 12 mo - n (%) | 21 (51.2) | 23 (59.0) |
| Hemoglobin - mean (SD) g/dl [NRR: females 12-16, males 13.6-18] | 8.69 (1.08) | 8.68 (0.89) |
| Reticulocyte count — mean (SD) ×10⁹ /I [NRR: 30-120] | 217.5 (75.0) | 216.2 (69.1) |
| Lactate dehydrogenase - mean (SD) U/I [NRR: 113-226] | 257.5 (97.6) | 308.6 (284.8) |
| Total bilirubin - mean (SD) µmol/I [NRR: 1.7-18.8] | 42.5 (31.5) | 40.5 (26.6) |
| Indirect bilirubin - mean (SD) µmol/I | 34.7 (28.5) | 32.9 (23.0) |
| FACIT-F score — mean (SD) | 32.2 (11.4) | 31.6 (12.5) |

FACIT-F, Functional Assessment of Chronic Illness Therapy-Fatigue; NRR, normal reference range; PNH, paroxysmal nocturnal hemoglobinuria.
^{a}One patient in the pegcetacoplan group received 900 mg eculizumab every 11 days

Additionally, 21 (51.2%) subjects in the APL-2 group had ≥ 4 transfusions in previous 12 months, and 23 (59.0%) subjects in the Eculizumab group had ≥ 4 transfusions in previous 12 months. The proportion of transfusion-free patients was similar in the pegcetacoplan group regardless of transfusion strata (85.0% vs 85.7% for <4 transfusions vs ≥4 transfusions, respectively) and greater than the eculizumab (ECU) group regardless of transfusion strata (31.3% vs 4.3% for <4 vs ≥4 transfusions). Further, the proportion of patients who were transfusion-free was similar in the pegcetacoplan group, regardless of platelet strata (83.3% vs 86.2% for <100,000 /mm³ vs ≥100,000 /mm³, respectively). In the ECU group no patients (0%) in the <100,000 /mm³ group and 20.0% of patients in the ≥100,000 /mm³ group were transfusion-free.

Subjects in the APL-2 group had a mean indirect bilirubin level of 34.7 µmol/L (28.5 SD), and subjects in the eculizumab group had a mean indirect bilirubin level of 32.9 µmol/L (23.0 SD).

TABLE 6

Select Medical History

| | APL-2 (N+41) | Eculizumab (N=39) | Total (N=80) |
|---|---|---|---|
| | n (%) | n (%) | n (%) |
| Aplastic Anemia | 11 (26.8) | 9 (23.1) | 20 (25) |
| Any Thrombosis* | 13 (31.7) | 10 (25.6) | 23 (28.8) |
| Thrombocytopenia | 1 (2.4) | 5 (12.8) | 6 (7.5) |
| Pancytopenia | 3 (7.3) | 1 (2.006) | 4 (5) |
| Agranulocytosis | 1 (2.4) | 0 | 1 (1.3) |

*Patients may have had more than 1 type of thromboembolic event

### Hemoglobin

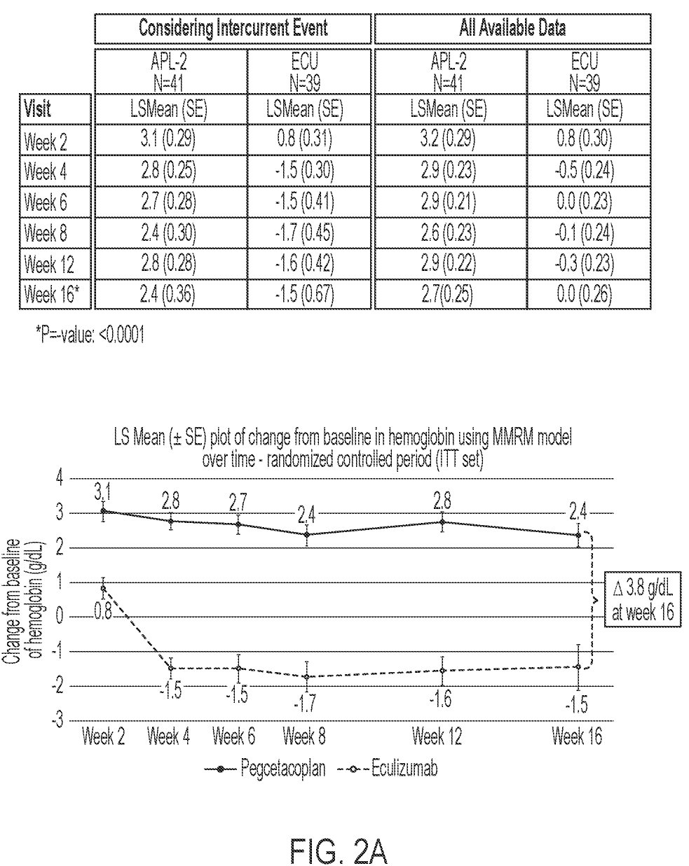

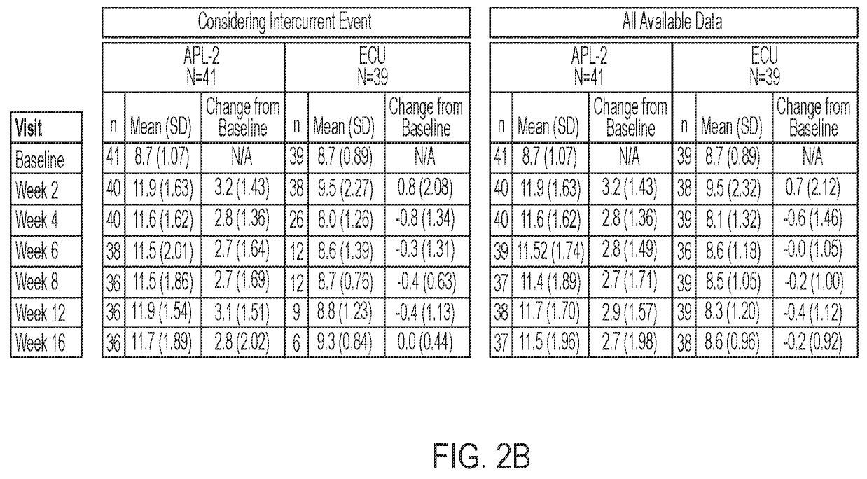

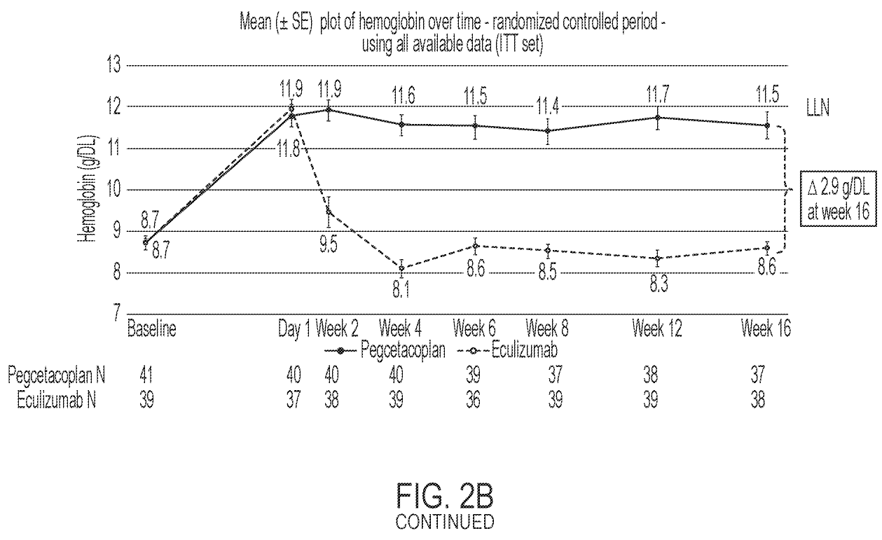

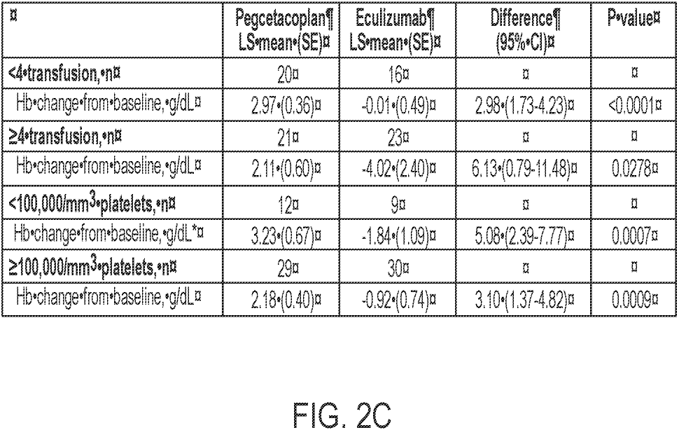

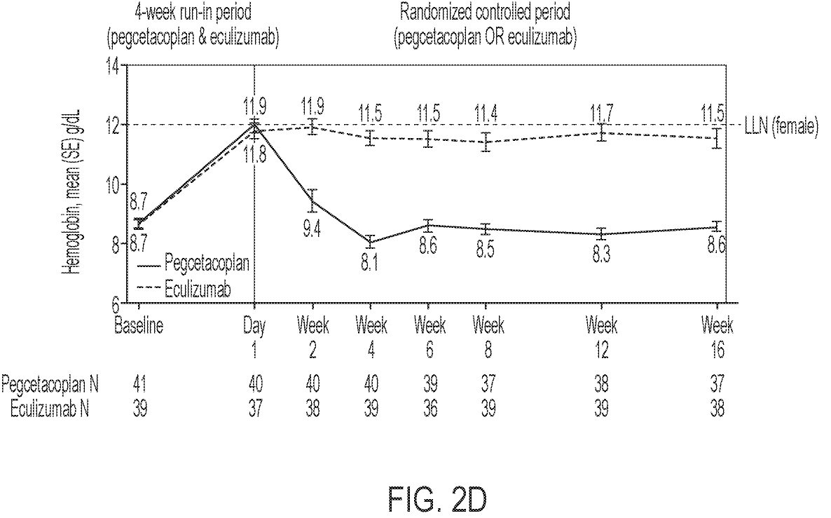

Top-line data demonstrated that pegcetacoplan met the study’s primary efficacy endpoint, demonstrating superiority to eculizumab with a statistically significant improvement in adjusted means of 3.8 g/dL of hemoglobin at week 16 (p<0.0001). At week 16, pegcetacoplan-treated subjects (n=41) had an adjusted mean hemoglobin increase of 2.4 g/dL from baseline of 8.7 g/dL, compared to eculizumab-treated subjects (n=39) who had a change of -1.5 g/dL from a baseline of 8.7 g/dL (see FIG. 2A). Pegcetacoplan increased hemoglobin independent of transfusion history. As shown in FIG. 2C, in patients with fewer than 4 transfusions within the 12 months prior to Day -28, pegcetacoplan-treated patients (n=20) had an adjusted mean hemoglobin increase of 2.97 g/dL vs. eculizumab-treated patients (n=16) who had a mean change of -0.01 g/dL from the 8.9 g/dL baseline. In patients with 4 or more transfusions within the 12 months prior to Day -28, pegcetacoplan-treated patients (n=21) had an adjusted mean hemoglobin increase of 2.11 g/dL vs. eculizumab-treated patients (n=23) who had a mean change of -4.02 g/dL from the 8.5 g/dL baseline (based on the pre-specified analysis, hemoglobin levels following transfusions were excluded to isolate the impact of the treatment from that of transfusions, which can otherwise artificially increase hemoglobin levels). Additionally, as shown in FIG. 2C, at week 16 regardless of baseline platelet count strata, mean hemoglobin significantly increased from baseline in the pegcetacoplan group and decreased in the eculizumab group.

Hemoglobin increase was maintained with pegcetacoplan at 16 weeks including post-transfusion data (see FIG. 2D, showing all available data in all patients regardless of transfusion events). (“Including post-transfusion data” and “all available data” are used interchangeably herein.) At week 16, a greater proportion of patients receiving pegcetacoplan achieved ≥2 g/dL improvement in hemoglobin (61% vs 0%), hemoglobin normalization (34% vs 0%), and hemoglobin stabilization (85% vs 15%) censored for transfusion as compared with eculizumab.

### Transfusion Avoidance and Absolute Reticulocyte Count

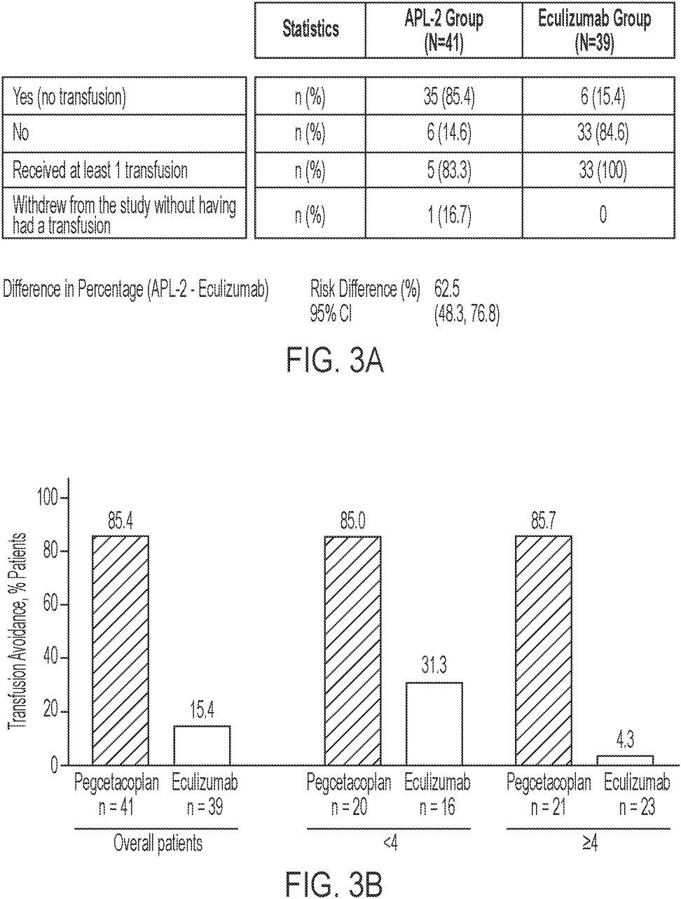

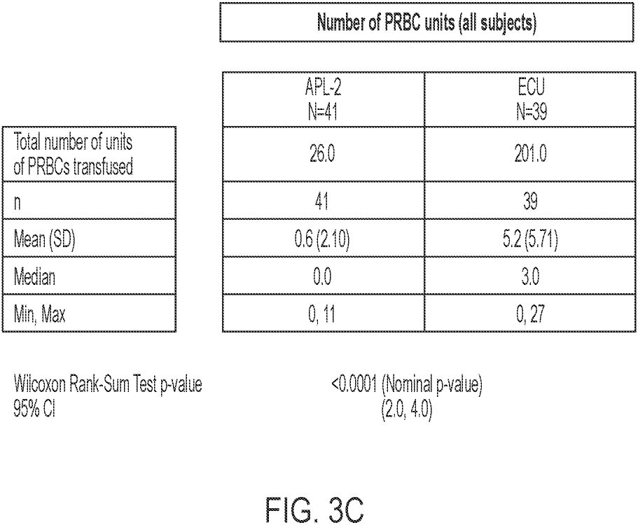

Non-inferiority was met in the key secondary endpoints of transfusion avoidance and absolute reticulocyte count. As shown in FIG. 3A, transfusion avoidance was shown in 35/41 (85.4%) subjects in the pegcetacoplan group as compared to 6/39 (15.4%) subjects in the eculizumab group. 33 subjects out of 39 in the eculizumab group required transfusions post randomization compared to only 6 out of 41 in the pegcetacoplan group. FIG. 3B shows effect of pegcetacoplan on transfusion avoidance (overall and transfusion strata). For overall patients, adjusted risk difference was 62.5% (95% CI, 48.3%-76.8%), demonstrating non-inferiority. Adjusted risk difference (95% CI) for <4 group was 53.8% (26.2%-81.3%), and for ≥4 group was 81.4% (64.2%-98.5%). As shown in FIG. 3B, pegcetacoplan reduced transfusion requirements consistently across the study population. Overall, 85% of pegcetacoplan-treated patients were transfusion-free over 16 weeks vs. 15% of eculizumab-treated patients. In patients with fewer than 4 transfusions within the 12 months prior to Day -28, 85% of pegcetacoplan-treated patients were transfusion-free compared to 31% of eculizumab-treated patients. In patients with 4 or more transfusions within the 12 months prior to Day -28, 86% of pegcetacoplan-treated patients were transfusion-free compared to 4% of eculizumab-treated patients.

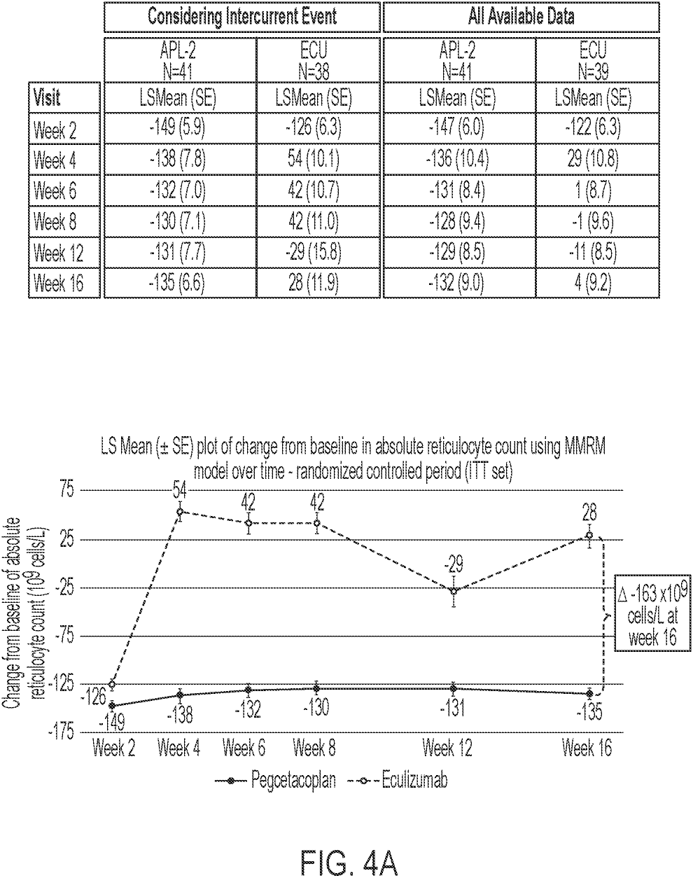

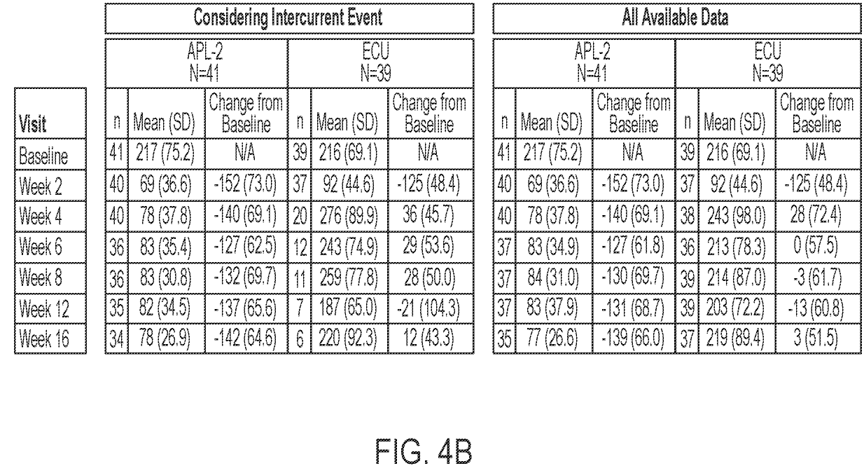

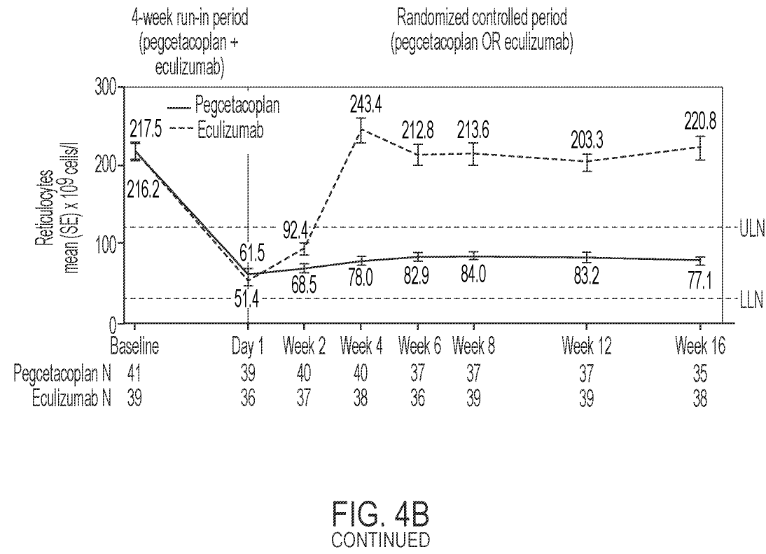

As shown in FIG. 4A, the change from baseline to week 16 in absolute reticulocyte count in the pegcetacoplan group had an adjusted mean decrease of 135×10⁹ /L from a baseline of 217×10⁹ /L, compared to the eculizumab group who had a mean increase of 28×10⁹ /L from a baseline of 216×10⁹ /L. Without wishing to be bound by theory, the decrease in the pegcetacoplan group may be the result of more complete control of hemolysis, which may reduce the burden on the bone marrow. Additionally, pegcetacoplan treatment was associated with significantly lower change from baseline in absolute reticulocyte count at week 16 compared to eculizumab, regardless of transfusion strata (absolute reticulocyte count LS mean change from baseline at week 16: -152.59 vs 22.06 × 10⁹ cells/L, P<0.0001, for <4 transfusions; -124.75 vs 39.26 × 10⁹ cells/L; P<0.0001, for ≥4 transfusions). The absolute reticulocyte count LS mean change from baseline was -147.17 × 10⁹ cells/L in the <100,000 /mm³ platelet stratum; no transfusion-free patients with uncensored data remained in this stratum in the eculizumab group at week 16. The absolute reticulocyte count LS mean change from baseline in the ≥100,000 /mm³ platelet stratum was -137.28 × 10⁹ cells/L with pegcetacoplan and 18.73 × 10⁹ cells/L with eculizumab.

### Lactate Dehydrogenase (LDH)

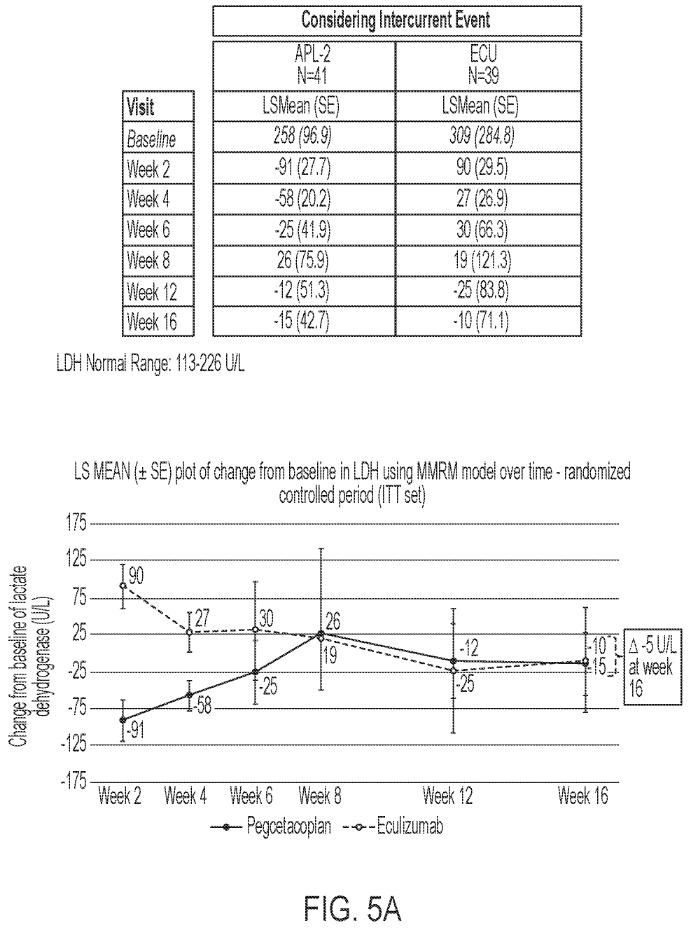

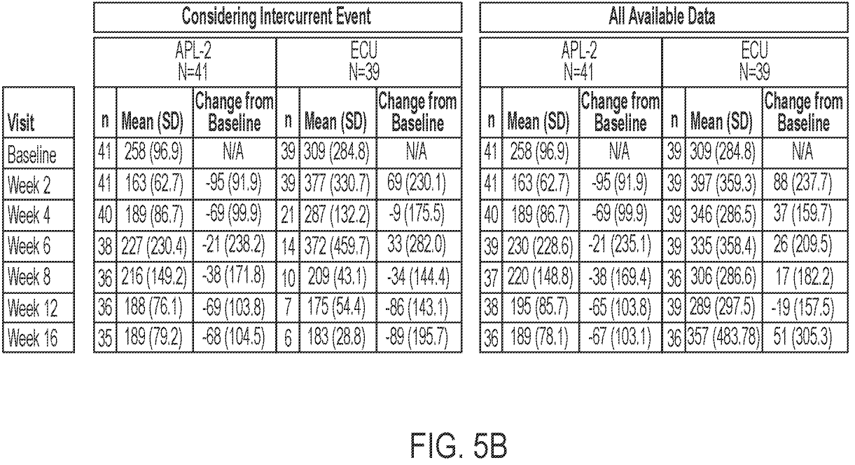

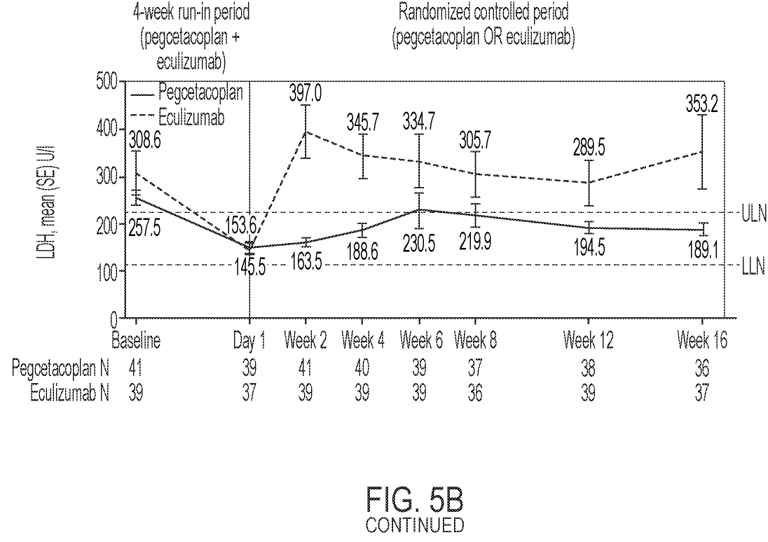

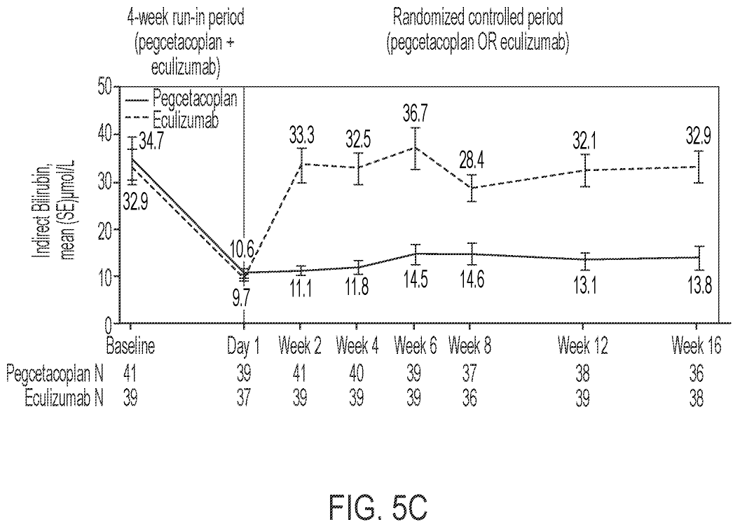

Pegcetacoplan did not demonstrate non-inferiority to eculizumab in the change from baseline in LDH at Week 16. As shown in FIG. 5A, the adjusted mean change from baseline was -15 U/L from a baseline of 258 U/L in the pegcetacoplan group as compared to a change of -10 U/L from a baseline of 309 U/L in the eculizumab group. The ability to demonstrate non-inferiority may have been limited by relatively controlled LDH due to prior eculizumab treatment and a slight imbalance between groups at baseline, as well as censoring of post-transfusion data. Additionally, LDH LS mean change from baseline at week 16 was -52.31 and -29.38 U/L for pegcetacoplan and eculizumab, respectively, in the <4 transfusions stratum, and -54.99 and 69.02 U/L, respectively, in the ≥4 transfusions stratum. At week 16 the LDH LS mean change from baseline in the <100,000 /mm³ platelet stratum was -73.14 U/L with pegcetacoplan; no uncensored data remained in this stratum in the eculizumab group. The LDH LS mean change from baseline in the ≥100,000 /mm³ platelet stratum was -41.96 U/L with pegcetacoplan and 28.47 U/L in the eculizumab group.

LDH is primarily a marker of intravascular hemolysis (IVH). Without intending to be limiting, it is believed that IVH is largely controlled in the presence of eculizumab, whereas APL-2 is believed to have increased hemoglobin, relative to eculizumab, primarily by inhibiting extravascular hemolysis (EVH) (in addition to IVH). Additionally, censoring of post-transfusion data reduced the amount of data used in the MMRM model, from week 6 onwards. Further, there was a large amount of inter- and intra-subject variation in LDH levels (see, e.g., the standard deviations and variation by study visit and error bars, especially in the eculizumab arm in FIG. 5A and FIG. 5B). Finally, the statistically significant difference in weeks 2 and 4 might have been due to increased IVH in the period immediately after the combination run-in period, and associated blood transfusions in the eculizumab arm in weeks 1-4.

### Indirect Bilirubin

As shown in FIG. 5C, indirect bilirubin level was 13.8 µmol/L at week 16 in pegcetacoplan subjects, and was 32.9 µmol/L at week 16 in eculizumab subjects.

### FACIT-Fatigue Score

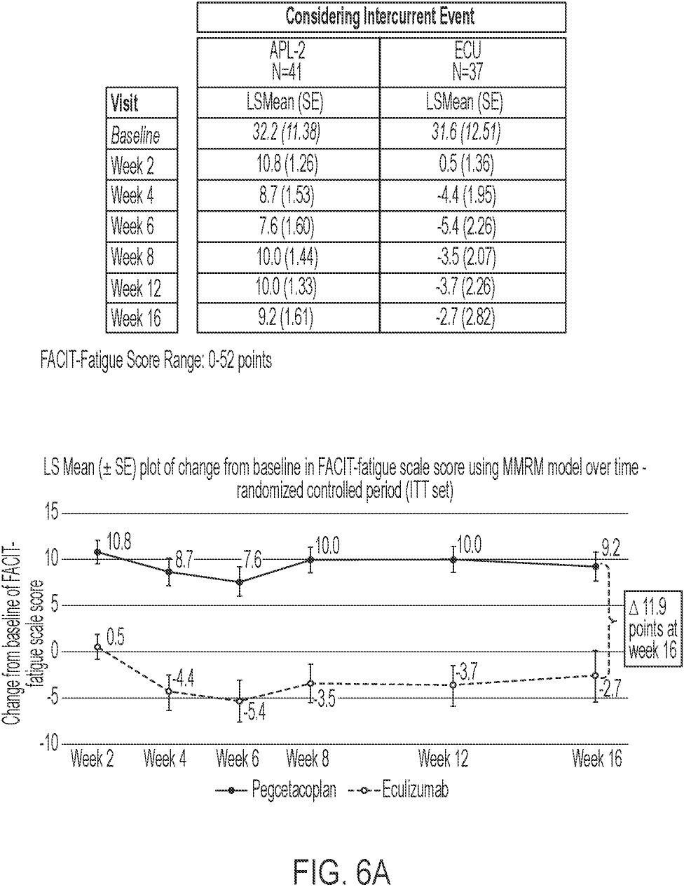

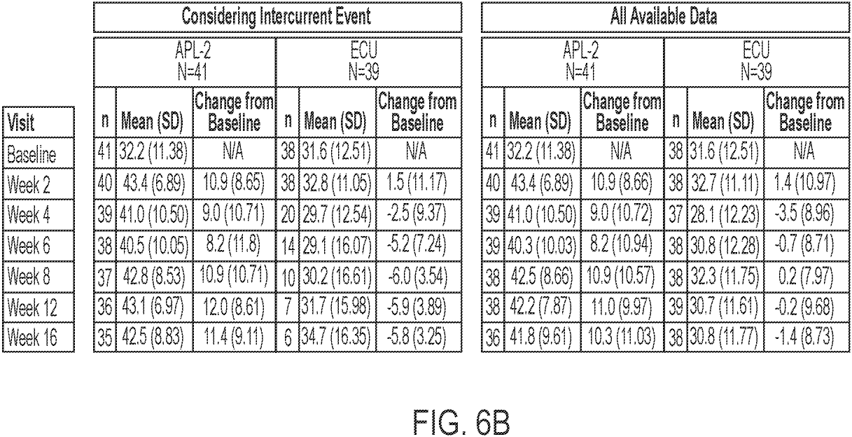

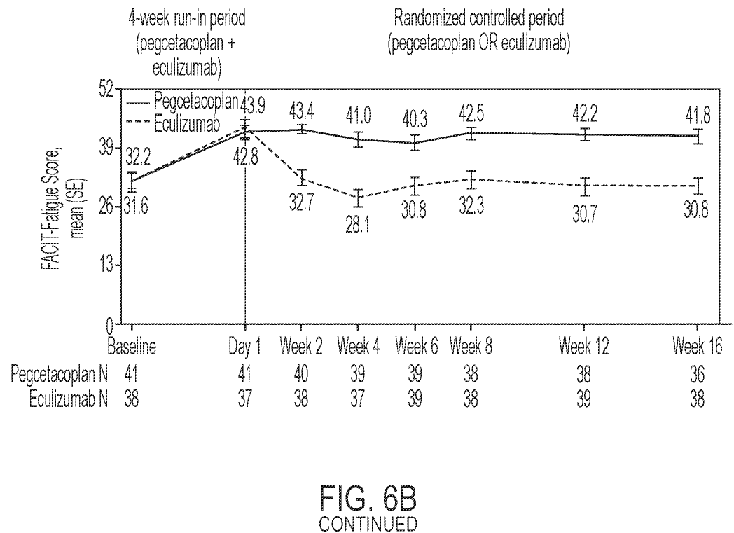

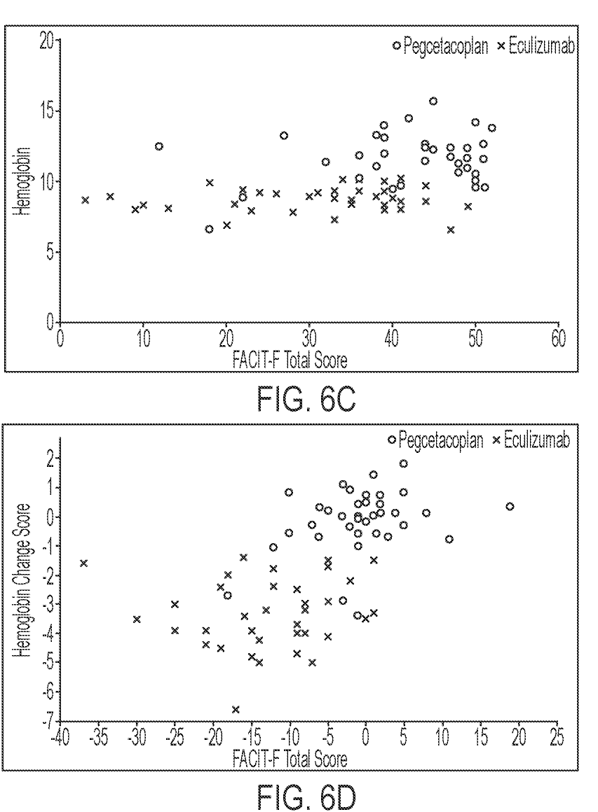

As shown in FIG. 6A, the adjusted mean change from baseline to Week 16 of the FACIT-fatigue score was 9.2 in the pegcetacoplan group and -2.7 in the eculizumab group. A 3-point change in FACIT-fatigue score is considered clinically meaningful. As shown in FIG. 6B, the mean baseline FACIT-fatigue score in the pegcetacoplan group was 32.2 (SD 11.38), which increased to 41.8 (SD 9.61) at week 16. As shown in FIG. 6B, the mean baseline FACIT-fatigue score in the eculizumab group was 31.6 (SD 12.51), and was 30.6 (SD 11.77) at week 16.

Post-hoc analyses were performed to explore relationships among effect modifiers, including fatigue and hemoglobin, reticulocyte count, indirect bilirubin, and physical functioning. Also, this study assessed further detail on the treatment effects of pegcetacoplan on fatigue in patients. Convergent validity was assessed using Spearman correlations and known groups validity was assessed using analysis of covariance (ANCOVA), as suggested by the FDA Guidance on patient-reported outcomes (Fed Regist. 2009; 74(235):65132-65133). Subjects were grouped based on hemoglobin level (<10 g/dL, 10 to ≤12 g/dL, ≥12 g/dL), and by degree of hemoglobin improvement: <1 g/dL, ≥1 to <2 g/dL, and ≥2 g/dL. At Week 16, in the overall sample (n= 80; intent-to-treat), the FACIT-F total score was significantly correlated with hemoglobin (r=0.48, p< 0.0001; see FIG. 6C), reticulocyte count (r=-0.37, p<0.01), indirect bilirubin (r=-0.25, p<0.05), and with multiple EORTC domains including fatigue and physical function (r=-0.87, p<0.0001 and r=0.78, p<0.0001 respectively). Reduction in fatigue (lower FACIT-F total scores) was associated with improvement in hemoglobin over 16 weeks (F= 11.0, p<0.0001), with the largest reduction in fatigue in the group with an increase in hemoglobin of ≥2 g/dL (10.7-point improvement in FACIT-F total score). In contrast, individuals with little to no hemoglobin improvement (<1 g/dL) reported slightly more fatigue (-2.5 worsening in FACIT-F total score). The FACIT-F also distinguished between groups with different levels of hemoglobin (<10 g/dL, 10 to ≤12 g/dL, >12 g/dL) at Week 16 (F= 5.39, p=0.0008). An analysis of the correlation of change scores indicates a clear separation between pegcetacoplan and eculizumab, showing a greater effect of pegcetacoplan on change in hemoglobin and improvement in fatigue (see FIG. 6D). In PNH, hemoglobin level and positive change in hemoglobin were significantly related to reduced fatigue. Pegcetacoplan resulted in significantly lower fatigue and hemoglobin scores at Week 16 compared to eculizumab.

### Type II + III PNH Red Blood Cells

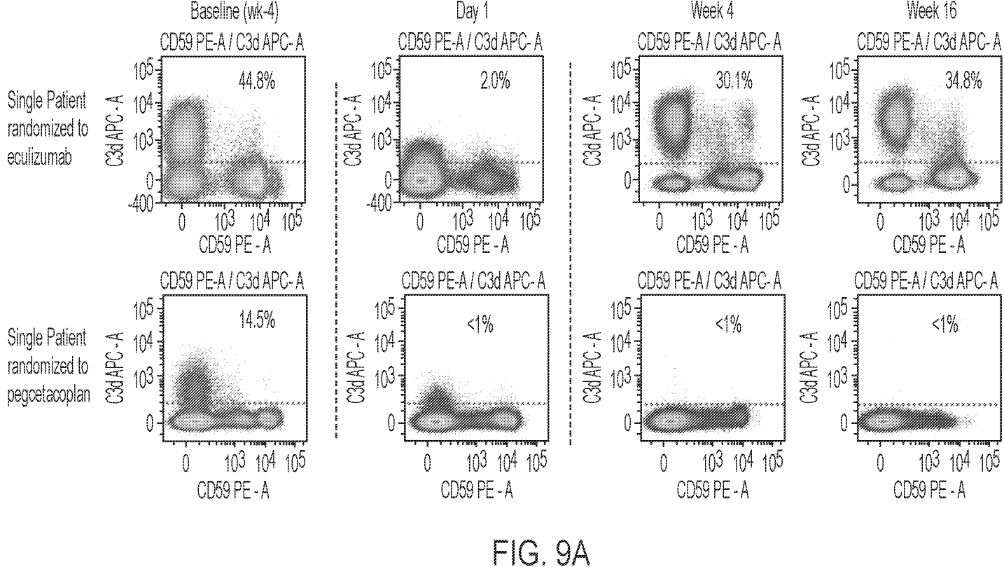

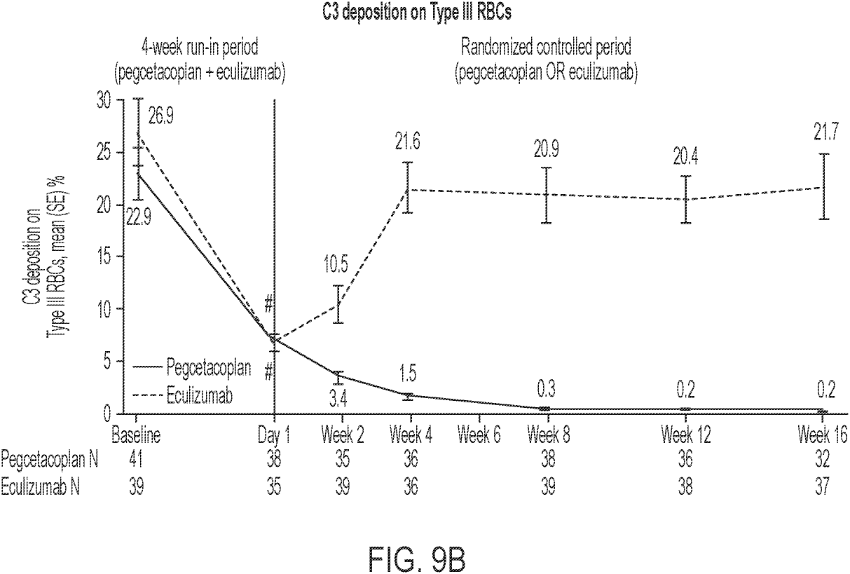

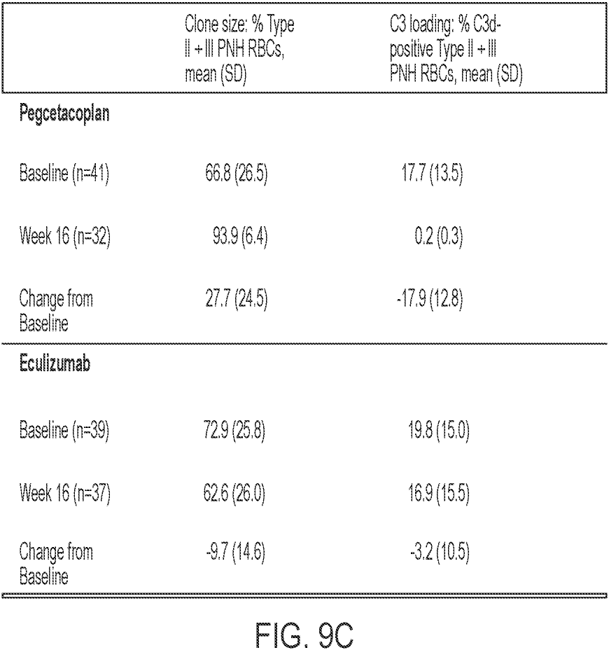

FIG. 9A shows C3d loading on red blood cells on a single pegcetacoplan and a single eculizumab subject. As shown in FIG. 9B, C3 loading on Type III RBCs was decreased in pegcetacoplan subjects at week 16. As shown in FIG. 9C, PNH clone size (Type II + III) was increased in pegcetacoplan subjects at week 16. FIGS. 9B and 9C includes descriptive analysis of observed values, based only on those subjects who had both baseline and week 16 data.

### Quality of Life

QoL assessments were Linear Analog Scale Assessment (LASA) and the European Organisation for Research and Treatment of Cancer Quality of Life Questionnaire-Core 30 Scale (EORTC QLQ-C30) scores. Change from baseline (CFB) to week 16 was analyzed using a mixed model for repeated measures. The LASA consists of 3 sections asking respondents to rate their perceived level of functioning and contains specific domains for activity level, ability to carry out daily activities, and overall QoL. Each section of the LASA is scored from low of 0 to high of 100 and asks patients to rate different aspects of their life over the past week; section 1 asks patients to rate their energy level, section 2 their ability to do daily activities, section 3 their overall quality of life. Scores for the 3 individual components of the scale and the combined score were included in the analysis. The EORTC contains 30 questions comprising 5 functional scale scores and individual items; it asks patients to answer 28 questions on a scale of 1 (“not at all”) to 4 (“very much”) that generally focus on the past week of their life. An additional 2 questions are rated on a scale of 1 (“very poor”) to 7 (“excellent”) for overall health and quality of life over the past week.

Eighty patients were included in the analysis (pegcetacoplan, n=41; ECU, n=39; intent to treat [ITT] set). Mean (SD) of the total of the 3 LASA scores were comparable at baseline for both treatment groups (pegcetacoplan, 161.0 [67.99]; ECU, 156.7 [61.27]). The difference in the least squares (LS) mean CFB in LASA scores using data censored for transfusion in the ITT set was 59.10 (95% CI: 16.88, 101.32) at week 16 for the comparison of pegcetacoplan with ECU. Results are provided in Table 7:

TABLE 7

| | Pegcetacoplan (n=41) LS mean (SE) | Eculizumab (n=39) LS mean (SE) | Difference (95% CI) in LS mean |
|---|---|---|---|
| LASA score | 49.4 (10.2) | -9.7 (19.0) | 59.1 (16.9, 101.3) |
| EORTC QLQ-C30 | | | |
| Global Health Status/QoL | 15.9 (3.6) | -2.7 (8.5) | 18.6 (0.1, 37.1) |
| Functional scales | | | |
| Physical functioning | 16.9 (2.1) | 4.1 (3.6) | 12.9 (4.9, 20.9) |
| Role functioning | 15.4 (3.9) | -9.0 (7.0) | 24.4 (8.8, 40.0) |
| Emotional functioning | 8.0 (3.4) | 3.9 (7.2) | 4.1 (-11.6, 19.8) |
| Cognitive functioning | 5.8 (3.3) | -3.8 (6.4) | 9.6 (-4.5, 23.6) |
| Social functioning | 15.1 (2.9) | 3.8 (6.3) | 11.3 (-2.4, 24.9) |
| Symptom scales | | | |
| Fatigue | -22.9 (3.3) | -2.2 (6.6) | -20.7 (-35.3, -6.2) |
| Nausea and vomiting | -0.3 (1.6) | -0.3 (3.9) | -0.0 (-8.4, 8.4) |
| Pain | -0.7 (4.3) | 2.0 (7.8) | -2.8 (-20.4, 14.9) |
| Dyspnea | -20.1 (3.5) | -5.6 (7.0) | -14.6 (-29.9, 0.8) |
| Insomnia | -9.2 (4.0) | -9.5 (7.1) | 0.3 (-15.7, 16.3) |
| Appetite loss | -3.8 (3.4) | 4.2 (7.0) | -8.0 (-23.2, 7.3) |
| Constipation | 3.0 (3.2) | 1.2 (8.1) | 1.8 (-15.7, 19.3) |
| Diarrhea | 0.3 (3.7) | 1.7 (8.2) | -1.4 (-19.3, 16.5) |
| Financial difficulties | -6.8 (3.9) | 0.6 (6.3) | -7.4 (-21.8, 7.0) |

*Baseline is the last available observation before first dose of pegcetacoplan. Model includes treatment + baseline value + analysis visit + strata + analysis visit x treatment, where strata is the combination of stratification factors: number of infusions and platelet count at screening.

EORTC QLQ-C30, European Organisation for Research and Treatment of Cancer Quality of Life Questionnaire-Core 30 Scale; ITT, intent to treat; LASA, Linear Analog Scale Assessment; LS, least square; SE, standard error.

The Global Health Status/QoL and all Functional Scales of the EORTC QLQ-C30 showed an improved score in the pegcetacoplan group at week 16, while the ECU group showed a mean decrease from baseline in the Global Health Status/QoL and role functioning scale score. Significant improvements in the Fatigue and Dyspnea scales were observed for pegcetacoplan compared with eculizumab (LS mean [95% CI] CFB: Fatigue, -20.7 [-35.3, -6.2]; Dyspnoea, -14.6 [-29.9, 0.8]). Compared with the ECU group, most of the symptom parameters were improved (negative values indicating improvement) in the pegcetacoplan group at week 16. Although no statistical tests for non-inferiority were performed on these QoL endpoints, substantial and clinically relevant improvements in QoL were consistently observed with pegcetacoplan compared with ECU at week 16 across both the LASA and EORTC QLQ-C30 scores.

### Hematologic Response to Treatment

Hematologic response to treatment was categorized (per Risitano AM, et al. Front Immunol. 2019; 10:1157) as complete, major, good, partial, minor, or no response using number of packed red blood cell transfusions required, hemoglobin (Hb) level, lactate dehydrogenase (LDH) level, and absolute reticulocyte count (ARC). Complete response: no transfusions required, stable Hb in the normal range, and no evidence of hemolysis (i.e., LDH ≤1.5× upper limit of normal [ULN], ARC ≤150,000 /µL). Major response: no transfusion, normal Hb, but with evidence of hemolysis (LDH >1.5×ULN and/or ARC >150,000 /µL). Good response: no transfusion, but with chronic mild anemia or evidence of hemolysis. Partial response: chronic moderate anemia and/or occasional transfusions (<3 units/6 months). Minor response: regular transfusions required (3-6 units/6 months). No response: regular and frequent transfusions required (>6 units/6 months).

The intent-to-treat (ITT) population included 41 patients randomized to pegcetacoplan and 39 patients randomized to eculizumab. 4 patients in the pegcetacoplan group and 1 patient in the eculizumab group were not evaluable for analysis due to incomplete data at week 16.

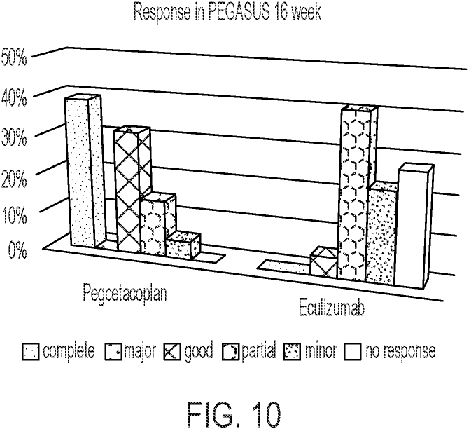

As shown in FIG. 10, at 16 weeks, the distribution of response categories was as follows: complete responses were 39% in the pegcetacoplan arm and 0% in the eculizumab arm, good responses 31.7% and 5.1% (pegcetacoplan and eculizumab, respectively), partial responses 14.6% and 41.0% (pegcetacoplan and eculizumab, respectively), minor responses 4.9% and 23.1% (pegcetacoplan and eculizumab, respectively), and no responses 0% and 28.2% (pegcetacoplan and eculizumab, respectively). Altogether, 29/41 patients (70.7%) in the pegcetacoplan arm achieved at least a good hematological response, in contrast to 2/39 (5.1%) of the eculizumab arm. Among the factors that may contribute to heterogeneity of hematologic response to treatment are impaired bone marrow function, residual intravascular hemolysis, and residual C3-mediated extravascular hemolysis. Bone marrow failure was ruled out, and no difference in LDH was observed, suggesting that the major factor accounting for the difference between the two arms was the prevention of C3-mediated EVH (as confirmed by reduction of C3-opsonization of PNH RBCs). This post-hoc analysis demonstrates that pegcetacoplan resulted in a significant shift toward better hematological responses in PNH patients, as compared with eculizumab. These results further support the concept that proximal complement inhibition, by preventing EVH in addition to controlling IVH, leads to meaningful improvement in the treatment of PNH.

### Safety

Pegcetacoplan was generally well tolerated. There were no deaths. 3 subjects randomized to the pegcetacoplan group discontinued study treatment due to a TEAE of hemolysis (one was SAE). Events occurred between week 4 and week 8 of the randomized control period. No complement-amplifying conditions were reported preceding or concurrent with the events. LDH increased to 3-11× ULN. 2 of the 3 subjects had lower than expected serum concentrations of pegcetacoplan prior to the hemolysis events. Neither patient increased dosing of pegcetacoplan to 1080 mg every 3 days prior to treatment discontinuation. Frequency of adverse events was similar between groups during the randomized, 16-week period, as depicted in Table 8 below.

TABLE 8

| | Pegcetacoplan N=41 | Eculizumab N=39 |
|---|---|---|
| | n(%) | n(%) |
| Overview | | |
| Any TEAE | 36 (87.8) | 34 (87.2) |
| Serious AE | 7 (17.1) | 5 (15.4) |
| Discontinuations due to AE | 3 (7.3) | 0 |
| Adverse Events of Interest | | |
| All Infections | 12 (29.3) | 9 (23.1) |
| Sepsis | 0 | 0 |
| Meningitis | 0 | 0 |
| Hemolysis | 4 (9.8) | 9 (23.1) |
| Injection Site Reactions | 15 (35.6) | 1 (2.6) |
| Other Frequent Adverse Events (n≥4) | | |
| Diarrhea | 9 (22.0) | 1 (2.5) |
| Headache | 3 (7.3) | 9 (23.1) |
| Fatigue | 2 (4.9) | 6 (15.4) |
| Abdominal Pain | 5 (12.2) | 4 (10.3) |
| Back Pain | 3 (7.3) | 4 (10.3) |
| Dizziness | 1 (2.4) | 4 (10.3) |

### Conclusions

The top-line data showed that pegcetacoplan met the trial’s primary efficacy endpoint, demonstrating superiority to eculizumab with a statistically significant improvement in adjusted means of 3.8 g/dL of hemoglobin at week 16 (p<0.0001), 53% higher than the eculizumab arm. At week 16, pegcetacoplan-treated patients (n=41) had an adjusted mean hemoglobin increase of 2.4 g/dL from a baseline of 8.7 g/dL, compared to eculizumab-treated patients (n=39) who had a change of -1.5 g/dL from a baseline of 8.7 g/dL. Improvement in hemoglobin was noted regardless of baseline transfusion strata.

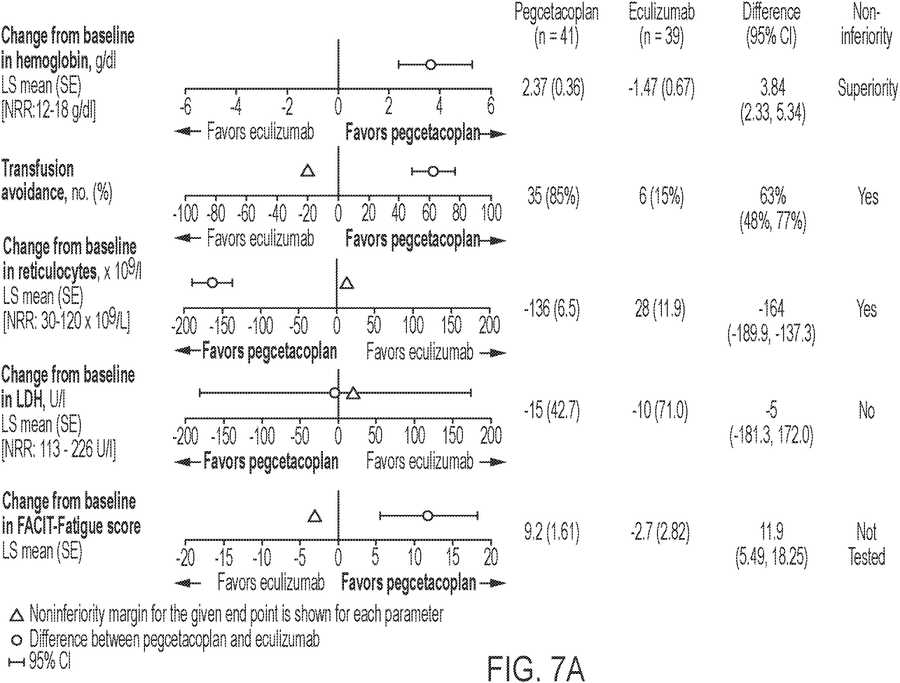

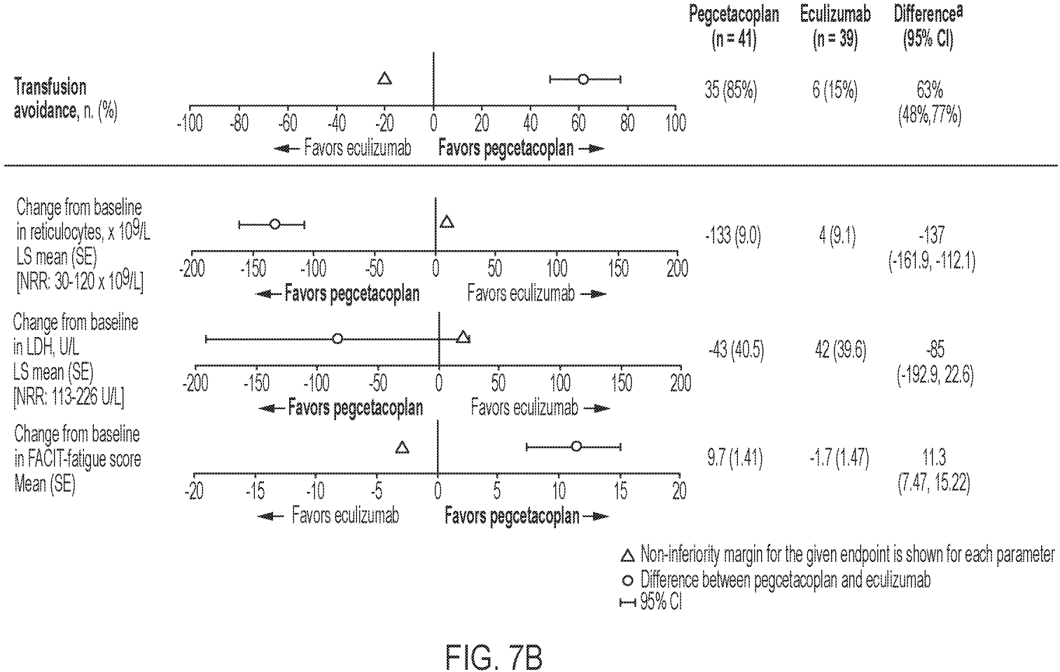

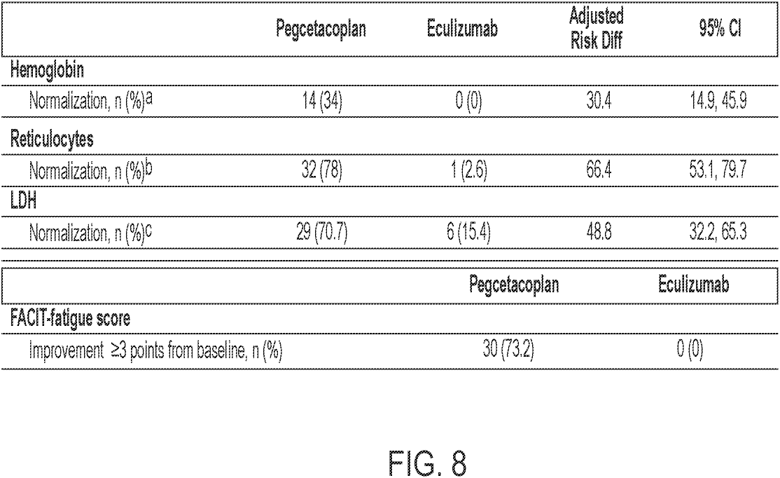

Additionally, pegcetacoplan showed promising results in key secondary endpoints. Pegcetacoplan met non-inferiority on transfusion avoidance and absolute reticulocyte count. Pegcetacoplan showed positive trends on mean lactate dehydrogenase, or LDH, and fatigue as measured by the Functional Assessment of Chronic Illness Therapy, or FACIT-fatigue score. Tables summarizing the results from the key secondary endpoints are depicted in FIGS. 7A and 7B (including post-transfusion data). Normalization of hematologic markers and clinically meaningful improvement on FACIT-fatigue score at 16 weeks are depicted in FIG. 8. As shown in FIG. 8, 78% of pegcetacoplan-treated patients achieved reticulocyte normalization vs. 3% of eculizumab-treated patients; 71% of pegcetacoplan-treated patients achieved LDH normalization vs. 15% of eculizumab-treated patients; and 73% of pegcetacoplan-treated patients achieved at least a three-point improvement in FACIT-fatigue score vs. 0% of eculizumab-treated patients (a three-point improvement in FACIT-fatigue score is generally considered to be clinically meaningful, see, e.g., Cella et al., J Pain Symptom Manage. 2002; 24(6):547-561; Nordin et al., BMC Med Res Methodol. 2016; 16:62).

The statistical analysis plan for the PEGASUS trial provided for use of the mixed model - repeated measures (MMRM) method. To avoid the effect of transfusions in hemoglobin levels during the 16-week randomization period of the trial, if a patient received a transfusion during the 16-week randomization period, any measurements after the first transfusion were censored from the data used in the MMRM analysis. The treatment effects using observed data from the trial, which included all post-transfusion measurements, were consistent with and supportive of the reported results from the MMRM analysis.

In the trial, the safety profile of pegcetacoplan was comparable to eculizumab. Seven of 41 patients (17.1%) in the pegcetacoplan group experienced a serious adverse event, or SAE, and 6 of 39 patients (15.4%) in the eculizumab group experienced SAEs. No cases of meningitis and no deaths were reported in either treatment group. The most common adverse events reported during the 16-week, randomized, controlled treatment period in the pegcetacoplan and eculizumab groups, respectively, were injection site reactions (36.6% vs. 2.6%), diarrhea (22.0% vs. 2.6%), headache (7.3% vs. 23.1%) and fatigue (4.9% vs. 15.4%). Another common adverse event was hemolysis, which was reported in four patients in the pegcetacoplan group (9.8%) and nine patients in the eculizumab group (23.1%). This led to the three discontinuations in the pegcetacoplan group.

All patients who completed the randomization period in both groups (77/80) entered the 32-week open-label pegcetacoplan treatment period.

### EXAMPLE 2 - Comparative Effectiveness of Pegcetacoplan Versus Ravulizumab in Patients with Paroxysmal Nocturnal Hemoglobinuria Previously Treated with Eculizumab: A Matching-Adjusted Indirect Comparison

We aimed to assess the comparative effectiveness of pegcetacoplan to ravulizumab through comparison of phase 3 study results, using matching-adjusted indirect comparison (MAIC) methodology, anchoring on the common comparator arm in the studies, eculizumab.

### Methods

Individual patient data from PEGASUS described in Example 1 (an ongoing, randomized, phase 3 study comparing pegcetacoplan and eculizumab among patients with PNH previously treated with eculizumab), were used to adjust for baseline differences compared to aggregate, published results from the randomized “302 study” (Kulasekararaj et al., 2019, Blood 133:540-549, PMID: 30510079), which compared ravulizumab and eculizumab among patients with PNH previously treated with eculizumab. Both studies share similar eligibility criteria. However, PEGASUS also required patients to have hemoglobin <10.5 g/dL and absolute reticulocyte count >1.0x the upper limit of normal; these criteria were not applicable in the 302 study. To adjust for cross-study differences in baseline characteristics, propensity score weighting was utilized to balance baseline demographic and clinical characteristics. Outcomes assessed included: transfusion avoidance, total number of units of packed red blood cells (PRBCs) transfused, hemoglobin stabilization, and change in Functional Assessment of Chronic Illness Therapy (FACIT)-Fatigue score. Outcomes were assessed from PEGASUS at Week 16 and from the 302 study at Week 26. Unadjusted mean and least squares mean change in FACIT-Fatigue score were compared for PEGASUS and the 302 study, respectively. Weighted Wald tests and 95% confidence intervals (CIs) were computed for comparisons of categorical and continuous outcomes (i.e., chi square and z tests, respectively).

### Conclusions

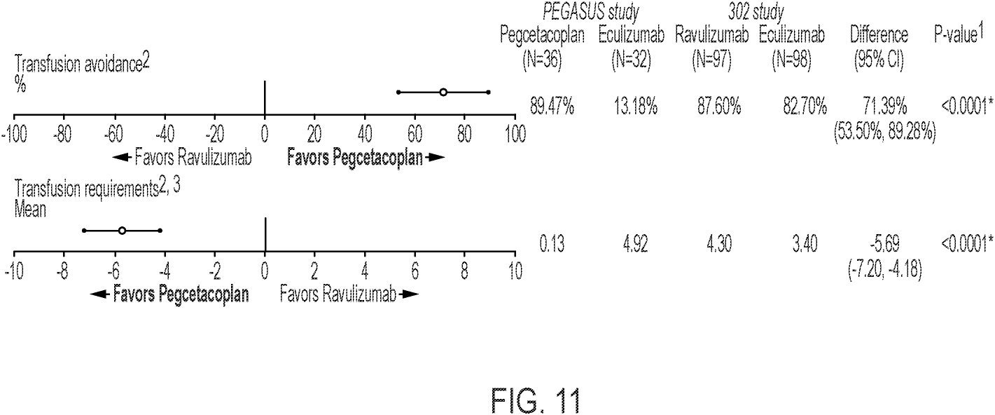

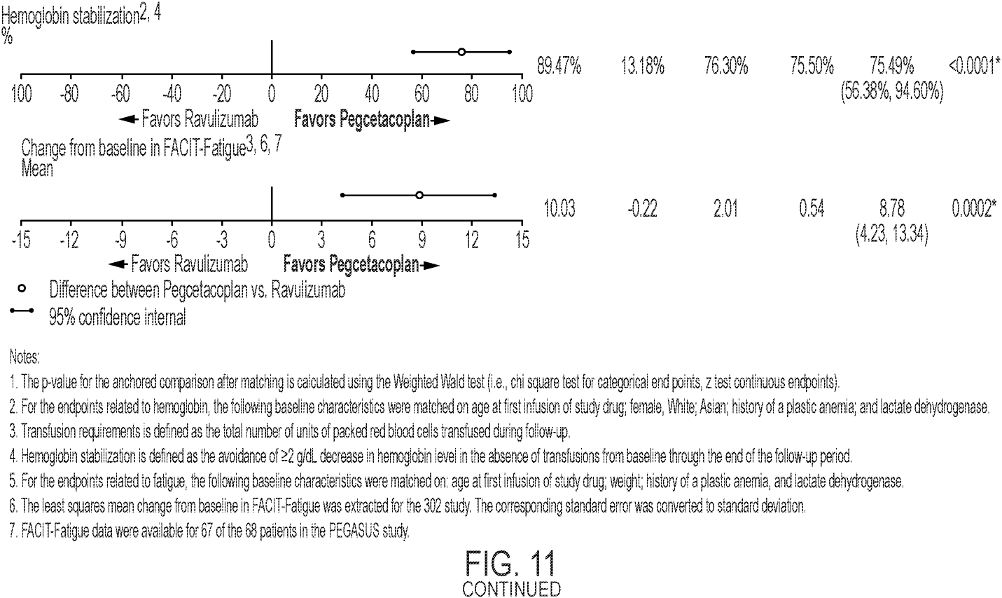

MAIC methodology allowed examination of the comparative effectiveness of pegcetacoplan vs. ravulizumab in the absence of a head-to-head trial. As shown in FIG. 11, results suggested an improvement in transfusion avoidance, hemoglobin stabilization, and fatigue, and a reduction in the total number of units of PRBCs transfused for patients who received pegcetacoplan, a C3 inhibitor, in PEGASUS, vs. patients who received ravulizumab, a C5 inhibitor, in the 302 study.

### Example 3 - PEGASUS (48-Week Study Results)

This Example describes 48-week results from the Phase 3 study described in Example 1.

### Methods

The methods of Example 1 were followed. A schematic of the PEGASUS trial as conducted is shown in FIG. 1B.

All patients (n=77) who completed the 16-week randomized controlled period of the PEGASUS study, which evaluated the Study Drug pegcetacoplan compared to eculizumab, entered the open-label period (OLP) and received the Study Drug from Week 17 to Week 48.

### Results

### Hemoglobin

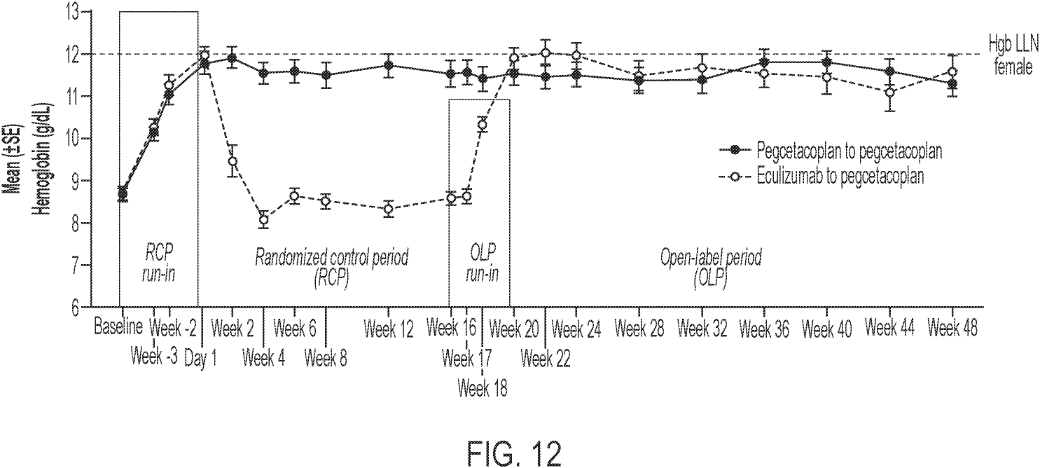

At Week 48, hemoglobin increases were sustained in pegcetacoplan-treated patients with a mean improvement from baseline of 2.7 g/dL, which is equal to the 2.7 g/dL mean increase seen at Week 16 with pegcetacoplan-treated patients. Additionally, eculizumab-treated patients who switched to pegcetacoplan during the open-label period experienced sustained improvements in hemoglobin and other hematological and clinical measures, similar to patients treated with pegcetacoplan monotherapy during the randomized controlled period. FIG. 12 shows the mean hemoglobin (g/dL) levels from baseline to week 48 (observed data over time) for the APL-2 group and the eculizumab group.

### Other Key Secondary Endpoints

In addition to a sustained improvement in hemoglobin, patients treated with pegcetacoplan maintained improvements across key secondary endpoints. Throughout the 48-week study, 73% of patients treated with pegcetacoplan remained transfusion free. For comparison, 25% of patients were transfusion free over the year prior to entering the PEGASUS study while on treatment with eculizumab. Improvements across additional markers of disease, such as reticulocyte count, lactate dehydrogenase (LDH) levels, and the Functional Assessment of Chronic Illness Therapy (FACIT)-fatigue scores, were maintained (see Example 1).

### Safety

Overall, the safety profile of pegcetacoplan throughout the 48-week study was consistent with data described in Example 1. Twenty-four of 80 pegcetacoplan monotherapy-treated patients (30%) experienced a serious adverse event (SAE); five of the SAEs (6%) were assessed to be possibly related to study treatment. No cases of meningitis were reported. One death was reported due to COVID-19 and was unrelated to study treatment. The most common adverse events (AEs) reported throughout the study were injection site reactions (36%), hemolysis (24%), and diarrhea (21%). Twelve out of 80 patients (15%) discontinued due to adverse events, with five discontinuations due to hemolysis. Sixty-four of the 67 patients (96%) who completed the open-label period opted to enter the extension study.

### Conclusions

Treatment with pegcetacoplan resulted in a sustained improvement in hemoglobin with a mean increase from baseline of 2.7 g/dL at Week 48, which is equal to the 2.7 g/dL increase seen at Week 16 with pegcetacoplan-treated patients. Sustained improvements in transfusion avoidance, reticulocyte count, lactate dehydrogenase (LDH) level, and Functional Assessment of Chronic Illness Therapy (FACIT)-fatigue score were observed in patients treated with pegcetacoplan. Safety profile of pegcetacoplan was consistent with previously reported data.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of treating a subject suffering from paroxysmal nocturnal hemoglobinuria (PNH), comprising subcutaneously administering to the subject pegcetacoplan, wherein:
(a) if the subject’s LDH level is less than or equal to twice the upper limit of normal, pegcetacoplan is administered in a 1080 mg dose twice weekly; and/or
(b) if the subject’s LDH level is greater than twice the upper limit of normal, pegcetacoplan is administered in a 1080 mg dose every three days.

2. A method of treating a subject suffering from paroxysmal nocturnal hemoglobinuria (PNH), comprising subcutaneously administering to the subject pegcetacoplan, wherein the patient is treated with a C5 inhibitor at a current dose before administration of a first dose of pegcetacoplan, wherein:
during the first four weeks of treatment, pegcetacoplan is administered in a 1080 mg dose twice weekly or every three days and the C5 inhibitor is administered at the current dose, and
after the first four weeks of treatment, pegcetacoplan is administered in a 1080 mg dose twice weekly or every three days and the administration of the C5 inhibitor is discontinued.

3. The method of claim 2, wherein the subject is transfusion-dependent at the current dose of the C5 inhibitor and before administration of the first dose of pegcetacoplan.

4. The method of claim 2 or claim 3, wherein the subject’s hemoglobin level is less than about 11 g/dL, less than about 10.5 g/dL, less than about 10 g/dL, less than about 9 g/dL, or less than about 8 g/dL, at the current dose of the C5 inhibitor and before administration of the first dose of pegcetacoplan.

5. The method of any one of claims 2–4, wherein the C5 inhibitor is an anti-C5 antibody.

6. The method of claim 5, wherein the anti-C5 antibody is eculizumab.

7. A method of treating a subject suffering from paroxysmal nocturnal hemoglobinuria (PNH), comprising subcutaneously administering to the subject 1080 mg of pegcetacoplan in a 20 mL solution twice weekly.

8. A method of treating a subject suffering from paroxysmal nocturnal hemoglobinuria (PNH), comprising subcutaneously administering to the subject 1080 mg of pegcetacoplan in a 20 mL solution every three days.

9. The method of any one of claims 2–8, wherein:
(a) if the subject’s LDH level is less than or equal to twice the upper limit of normal, pegcetacoplan is administered in a 1080 mg dose twice weekly; and/or
(b) if the subject’s LDH level is greater than twice the upper limit of normal, pegcetacoplan is administered in a 1080 mg dose every three days.

10. The method of claim 1 or claim 9, wherein the subject initially is administered pegcetacoplan in a 1080 mg dose twice weekly, and if during the treatment, the subject’s LDH level is assessed to be greater than twice the upper limit of normal, the subject subsequently is administered pegcetacoplan in a 1080 mg dose every three days.

11. The method of claim 1, claim 9 or claim 10, wherein if the subject is administered pegcetacoplan in a 1080 mg dose every three days after exhibiting an LDH level greater than twice the upper limit of normal, the method further comprises having the subject’s LDH level assessed twice weekly for at least two weeks.

12. The method of claim 1 or any one of claims 9–11, wherein the upper limit of normal is about 225 U/L.

13. The method of any one of claims 1–12, wherein pegcetacoplan is administered for at least about 12 weeks, about 16 weeks, about 20 weeks, about 24 weeks, about 28 weeks, about 32 weeks, about 36 weeks, about 40 weeks, about 44 weeks, about 48 weeks, or about 52 weeks.

14. The method of any one of claims 1–13, wherein pegcetacoplan is self-administered using a pump.

15. The method of any one of claims 1–14, wherein, following administration of pegcetacoplan, the subject’s hemoglobin level is increased to a target hemoglobin level.

16. The method of claim 15, wherein the target hemoglobin level is higher than a control hemoglobin level by at least 1 g/dL, e.g., by at least about 2 g/dL, e.g., by at least 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 g/dL, wherein the control hemoglobin level is:
(a) a hemoglobin level in the subject before administration of pegcetacoplan;
(b) a hemoglobin level in a subject suffering from PNH and not being administered pegcetacoplan; or
(c) a lower limit of a range of hemoglobin levels in a healthy subject.

17. The method of claim 15, wherein the target hemoglobin level is a hemoglobin level that is higher than a control hemoglobin level by at least about 20%, 40%, 60%, 80%, 100%, or more, wherein the control hemoglobin level is:
(a) a hemoglobin level in the subject before administration of pegcetacoplan;
(b) a hemoglobin level in a subject suffering from PNH and being administered pegcetacoplan; or
(c) a lower limit of a range of hemoglobin levels in a healthy subject.

18. The method of any one of claims 15–17, wherein the target hemoglobin level is about 11 g/dL to about 12 g/dL.

19. The method of claim 15, wherein the target hemoglobin level is at least 2 g/dL higher, e.g., about 2.4 g/dL higher, than a hemoglobin level in the subject before administration of pegcetacoplan.

20. The method of claim 18 or claim 19, wherein the target hemoglobin level is sustained for at least 16 weeks after the subject’s first dose of pegcetacoplan.

21. The method of any one of claims 1–20, further comprising measuring or having measured the subject’s hemoglobin level before and/or after administration of pegcetacoplan.

22. The method of any one of claims 15–21, wherein the subject’s hemoglobin level is increased in the absence of a transfusion.

23. The method of any one of claims 1–22, wherein, following administration of pegcetacoplan, the subject’s number of transfusions is reduced to a target number of transfusions.

24. The method of claim 23, wherein the target number of transfusions is at least 1 (e.g., at least 2, 3, 4, 5, 6 or more) fewer transfusions over a defined period of time relative to a control number of transfusions, wherein the control number of transfusions is:
(a) a number of transfusions administered to the subject before administration of pegcetacoplan; or
(b) a number of transfusions administered to a subject suffering from PNH and not being administered pegcetacoplan.

25. The method of claim 23 or claim 24, wherein the target number of transfusions is fewer than 3, 2, or 1 transfusions over about 4 weeks, 8 weeks, 12 weeks, 16 weeks, 20 weeks, 24 weeks, or more.

26. The method of claim 25, wherein the target number of transfusions is zero transfusions over about 4 weeks, 8 weeks, 12 weeks, 16 weeks, 20 weeks, 24 weeks, or more.

27. The method of any one of claims 1–26, comprising assessing or having assessed the need for administering a transfusion to the subject before and/or after administration of pegcetacoplan.

28. The method of any one of claims 1–27, wherein, following administration of pegcetacoplan, the subject’s number of administered packed red blood cell (PRBC) units is reduced to a target number of PRBC units.

29. The method of claim 28, wherein the target number of PRBC units is at least 1 (e.g., at least 2, 3, 4, 5, 6 or more) fewer transfusions over a defined period of time relative to a control number of PRBC units, wherein the control number of PRBC units is:
(a) a number of PRBC units administered to the subject before administration of pegcetacoplan; or
(b) a number of PRBC units administered to a subject suffering from PNH and not being administered pegcetacoplan.

30. The method of claim 28 or claim 29, wherein the target number of PRBC units is fewer than 3, 2, or 1 PRBC units over about 4 weeks, 8 weeks, 12 weeks, 16 weeks, 20 weeks, 24 weeks, or more.

31. The method of any one of claims 1–30, comprising assessing or having assessed the need for administering a PRBC unit to the subject before and/or after administration of pegcetacoplan.

32. The method of any one of claims 1–31, wherein, following administration of pegcetacoplan, the subject’s reticulocyte level is reduced to a target reticulocyte level.

33. The method of claim 32, wherein the target reticulocyte level is a reticulocyte level that is lower than a control reticulocyte level by at least about 20%, 40%, 60%, or 80%, wherein the control reticulocyte level is:
(a) a reticulocyte level in the subject before administration of pegcetacoplan;
(b) a reticulocyte level in a subject suffering from PNH and not being administered pegcetacoplan; or
(c) an upper limit of a range of reticulocyte levels in a healthy subject.

34. The method of claim 32, wherein the target reticulocyte level is about 30 × 10⁹/L to about 120 × 10⁹/L.

35. The method of claim 33, wherein the target reticulocyte level is about 30 × 10⁹ /L to about 100 × 10⁹ /L, e.g., about 70, 80, or 90 × 10⁹ /L.

36. The method of claim 34, wherein the target reticulocyte level is about 60 to 85 × 10⁹ /L, e.g., about 70 to 80 × 10⁹ /L.

37. The method of claim 32, wherein the target reticulocyte level is about 135 × 10⁹ /L lower than a reticulocyte level in the subject before administration of pegcetacoplan.

38. The method of claim 36 or claim 37, wherein the target reticulocyte level is sustained for at least 16 weeks after the subject’s first dose of pegcetacoplan.

39. The method of any one of claims 32–38, wherein, following administration of pegcetacoplan, the subject’s reticulocyte level is normalized.

40. The method of any one of claims 1–39, further comprising measuring or having measured the subject’s reticulocyte level before and/or after administration of pegcetacoplan.

41. The method of any one of claims 32–40, wherein the subject’s reticulocyte level is reduced and/or normalized in the absence of a transfusion.

42. The method of any one of claims 1–41, wherein, following administration of pegcetacoplan, the subject’s lactose dehydrogenase (LDH) level is reduced to a target LDH level.

43. The method of claim 42, wherein the target LDH level is an LDH level that is lower than a control LDH level by at least about 20%, 40%, 60%, or 80%, wherein the control LDH level is:
(a) an LDH level in the subject before administration of pegcetacoplan;
(b) an LDH level in a subject suffering from PNH and not being administrated pegcetacoplan; or
(c) an upper limit of a range of reticulocyte levels in a healthy subject.

44. The method of claim 42, wherein the target LDH level is about 110 to about 225 U/L, e.g., about 120, 140, 160, 180, 200, or 220 U/L.

45. The method of claim 42, wherein the target LDH level is about 160 to 230 U/L, e.g., about 190 U/L.

46. The method of claim 42, wherein the target LDH level is about 15 U/L lower than an LDH level in the subject before administration of pegcetacoplan.

47. The method of any one of claims 44–46, wherein the target LDH level is sustained for at least 16 weeks after the subject’s first dose of pegcetacoplan.

48. The method of any one of claims 42–47, wherein, following administration of pegcetacoplan, the subject’s LDH level is normalized.

49. The method of any one of claims 1–48, further comprising measuring or having measured the subject’s LDH level before and/or after administration of pegcetacoplan.

50. The method of any one of claims 42–49, wherein the subject’s LDH level is reduced and/or normalized in the absence of a transfusion.

51. The method of any one of claims 1–50, wherein, following administration of pegcetacoplan, the subject’s fatigue level is reduced to a target fatigue level.

52. The method of claim 51, wherein fatigue level is assessed using a FACIT-fatigue scale score.

53. The method of claim 51 or claim 52, wherein the target fatigue level is a FACIT-fatigue scale score that is higher than a control FACIT-fatigue scale score by at least 5, 10, 15, 20, or more points, wherein the control FACIT-fatigue scale score is:
(a) a FACIT-fatigue scale score from the subject before administration of pegcetacoplan;
(b) a FACIT-fatigue scale score from a subject suffering from PNH and not being administered pegcetacoplan; or
(c) a lower limit of a range of FACIT-fatigue scale scores from a healthy subject.

54. The method of any one of claims 51–53, wherein the target fatigue level is a FACIT-fatigue scale score of about 32, 34, 36, 38, 40, 42, 44, 46, or 48.

55. The method of claim 54, wherein the target fatigue level is a FACIT-fatigue scale score of about 40 to about 44.

56. The method of claim 53, wherein the target fatigue level is a FACIT-fatigue scale score that is about 7.5 to about 11 points higher, e.g., about 9 points higher, than a FACIT-fatigue scale score from the subject before administration of pegcetacoplan.

57. The method of claim 55 or claim 56, wherein the target fatigue level is sustained for at least 16 weeks after the subject’s first dose of pegcetacoplan.

58. The method of any one of claims 1–57, further comprising assessing or having assessed the subject’s FACIT-fatigue scale score before and/or after administration of pegcetacoplan.

59. The method of any one of claims 51–58, wherein the subject’s FACIT-fatigue scale score is increased in the absence of a transfusion.

60. A method of increasing level of hemoglobin, in a subject suffering from paroxysmal nocturnal hemoglobinuria (PNH), to a target hemoglobin level, the method comprising subcutaneously administering to the subject about 1080 mg pegcetacoplan twice weekly or every three days, thereby increasing hemoglobin in the subject to the target hemoglobin level.

61. The method of claim 60, wherein the target hemoglobin level is a hemoglobin level that is higher, relative to a control hemoglobin level, by at least about 1 g/dL, e.g., by at least about 2 g/dL, e.g., by at least 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 g/dL.

62. The method of claim 60, wherein the target hemoglobin level is a hemoglobin level that is higher, relative to a control hemoglobin level, by at least about 20%, 40%, 60%, 80%, 100%, or more.

63. The method of claim 61 or 62, wherein the control hemoglobin level is a hemoglobin level in a subject suffering from PNH and not receiving pegcetacoplan; a hemoglobin level in the subject before receiving pegcetacoplan; or a lower limit of a range of hemoglobin levels in a healthy subject.

64. The method of claim 60, wherein the target hemoglobin level is about 10 g/dL to about 15 g/dL, e.g., about 11 g/dL, about 12 g/dL, or about 13 g/dL.

65. The method of any one of claims 60–64, further comprising measuring hemoglobin level in the subject.

66. The method of claim 65, comprising measuring hemoglobin level in the subject before and/or after administration of pegcetacoplan.

67. The method of any one of claims 60–66, wherein hemoglobin is increased in the subject in the absence of transfusion.

68. A method of treating a subject suffering from PNH, the method comprising subcutaneously administering to the subject about 1080 mg pegcetacoplan twice weekly or every three days, wherein the treatment increases hemoglobin in the subject to a target hemoglobin level.

69. The method of claim 68, wherein the target hemoglobin level is a hemoglobin level that is higher, relative to a control hemoglobin level, by at least about 1 g/dL, e.g., by at least about 2 g/dL, e.g., by at least 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 g/d L.

70. The method of claim 68, wherein the target hemoglobin level is a hemoglobin level that is higher, relative to a control hemoglobin level, by at least about 20%, 40%, 60%, 80%, 100%, or more.

71. The method of claim 69 or 70, wherein the control hemoglobin level is a hemoglobin level in a subject suffering from PNH and not receiving pegcetacoplan; a hemoglobin level in the subject before receiving pegcetacoplan; or a lower limit of a range of hemoglobin levels in a healthy subject.

72. The method of claim 68, wherein the target hemoglobin level is about 10 g/dL to about 15 g/dL, e.g., about 11 g/dL, about 12 g/dL, or about 13 g/dL.

73. The method of any one of claims 68–72, further comprising measuring hemoglobin level in the subject.

74. The method of claim 73, comprising measuring hemoglobin level in the subject before and/or after administration of pegcetacoplan.

75. The method of any one of claims 68–74, wherein the subject is treated in the absence of a transfusion.

76. A method of reducing number of transfusions, administered to a subject in need thereof, to a target number of transfusions, the method comprising subcutaneously administering to the subject about 1080 mg pegcetacoplan twice weekly or every three days, thereby reducing number of transfusions to the target number of transfusions, and wherein the subject suffers from PNH.

77. The method of claim 76, wherein the target number of transfusions is at least 1 (e.g., at least 2, 3, 4, 5, 6 or more) fewer transfusions over a defined period of time relative to a control number of transfusions.

78. The method of claim 77, wherein the control number of transfusions is a number of transfusions administered to a subject suffering from PNH and not receiving pegcetacoplan; or a number of transfusions administered to the subject before receiving pegcetacoplan.

79. The method of claim 76, wherein the target number of transfusions fewer than 3, 2, or 1 transfusions over about 4 weeks, 8 weeks, 12 weeks, 16 weeks, 20 weeks, 24 weeks, or more.

80. The method of any one of claims 76–79, further comprising assessing the need for administering a transfusion to the subject.

81. The method of claim 80, comprising assessing the need for administering a transfusion to the subject before and/or after administration of pegcetacoplan.

82. A method of reducing number of PRBC units, administered to a subject in need thereof, to a target number of PRBC units, the method comprising subcutaneously administering to the subject about 1080 mg pegcetacoplan twice weekly or every three days, thereby reducing number of PRBC units to the target number of PRBC units, and wherein the subject suffers from PNH.

83. The method of claim 82, wherein the target number of PRBC units is at least 1 (e.g., at least 2, 3, 4, 5, 6 or more) fewer PRBC units administered over a defined period of time relative to a control number of PRBC units.

84. The method of claim 83, wherein the control number of PRBC units is a number of PRBC units administered to a subject suffering from PNH and not receiving pegcetacoplan; or a number of PRBC units administered to the subject before receiving pegcetacoplan.

85. The method of claim 82, wherein the target number of PRBC units is fewer than 3, 2, or 1 PRBC units over about 4 weeks, 8 weeks, 12 weeks, 16 weeks, 20 weeks, 24 weeks, or more.

86. The method of any one of claims 82–85, further comprising assessing the need for administering a PRBC unit to the subject.

87. The method of claim 86, comprising assessing the need for administering a PRBC unit to the subject before and/or after administration of pegcetacoplan.

88. A method of treating a subject suffering from PNH, the method comprising subcutaneously administering to the subject about 1080 mg pegcetacoplan twice weekly or every three days, wherein the treatment reduces the number of transfusions, administered to the subject, to a target number of transfusions.

89. The method of claim 88, wherein the target number of transfusions is at least 1 (e.g., at least 2, 3, 4, 5, 6 or more) fewer transfusions over a defined period of time relative to a control number of transfusions.

90. The method of claim 89, wherein the control number of transfusions is a number of transfusions administered to a subject suffering from PNH and not receiving pegcetacoplan; or a number of transfusions administered to the subject before receiving pegcetacoplan.

91. The method of claim 88, wherein the target number of transfusions is fewer than 3, 2, or 1 transfusions over about 4 weeks, 8 weeks, 12 weeks, 16 weeks, 20 weeks, 24 weeks, or more.

92. The method of any one of claims 88–91, further comprising assessing the need for administering a transfusion to the subject.

93. The method of claim 92, comprising assessing the need for administering a transfusion to the subject before and/or after administration of pegcetacoplan.

94. A method of treating a subject suffering from PNH, the method comprising subcutaneously administering to the subject about 1080 mg pegcetacoplan twice weekly or every three days, wherein the treatment reduces number of PRBC units, administered to the subject, to a target number of PRBC units.

95. The method of claim 94, wherein the target number of PRBC units is at least 1 (e.g., at least 2, 3, 4, 5, 6 or more) fewer PRBC units administered over a defined period of time relative to a control number of PRBC units.

96. The method of claim 95, wherein the control number of PRBC units is a number of PRBC units administered to a subject suffering from PNH and not receiving pegcetacoplan; or a number of PRBC units administered to the subject before receiving pegcetacoplan.

97. The method of claim 94, wherein the target number of PRBC units is fewer than 3, 2, or 1 PRBC units over about 4 weeks, 8 weeks, 12 weeks, 16 weeks, 20 weeks, 24 weeks, or more.

98. The method of any one of claims 94–97, further comprising assessing the need for administering a PRBC unit to the subject.

99. The method of claim 98, comprising assessing the need for administering a PRBC unit to the subject before and/or after administration of pegcetacoplan.

100. A method of reducing number of reticulocytes, in a subject suffering from PNH, to a target reticulocyte level, the method comprising subcutaneously administering to the subject about 1080 mg pegcetacoplan twice weekly or every three days, thereby reducing number of reticulocytes in the subject to the target reticulocyte level.

101. The method of claim 100, wherein the target reticulocyte level is a reticulocyte level that is lower, relative to a control reticulocyte level, by at least about 20%, 40%, 60%, or 80%.

102. The method of claim 101, wherein the control reticulocyte level is a reticulocyte level in a subject suffering from PNH and not receiving pegcetacoplan; a reticulocyte level in the subject before receiving pegcetacoplan; or an upper limit of a range of reticulocyte levels in a healthy subj ect.

103. The method of claim 100, wherein the target reticulocyte level is about 30 to about 100 X 10⁹ /L, e.g., about 70, 80, or 90 × 10⁹ /L.

104. The method of any one of claims 100–103, further comprising measuring reticulocyte level in the subject.

105. The method of claim 104, comprising measuring reticulocyte level in the subject before and/or after administration of pegcetacoplan.

106. The method of any one of claims 100–105, wherein number of reticulocytes is decreased in the subject in the absence of transfusion.

107. A method of treating a subject suffering from PNH, the method comprising subcutaneously administering to the subject about 1080 mg pegcetacoplan twice weekly or every three days, wherein the treatment decreases number of reticulocytes in the subject to a target reticulocyte level.

108. The method of claim 107, wherein the target reticulocyte level is a reticulocyte level that is lower, relative to a control reticulocyte level, by at least about 20%, 40%, 60%, or 80%.

109. The method of claim 108, wherein the control reticulocyte level is a reticulocyte level in a subject suffering from PNH and not receiving pegcetacoplan; a reticulocyte level in the subject before receiving pegcetacoplan; or an upper limit of a range of reticulocyte levels in a healthy subject.

110. The method of claim 107, wherein the target reticulocyte level is about 30 to about 100 × 10⁹ /L, e.g., about 70, 80, or 90 × 10⁹ /L.

111. The method of any one of claims 107–110, further comprising measuring reticulocyte level in the subject.

112. The method of claim 111, comprising measuring reticulocyte level in the subject before and/or after administration of pegcetacoplan.

113. The method of any one of claims 107–112, wherein the subject is treated in the absence of a transfusion.

114. A method of reducing lactate dehydrogenase (LDH) level, in a subject suffering from PNH, to a target LDH level, the method comprising subcutaneously administering to the subject about 1080 mg pegcetacoplan twice weekly or every three days, thereby reducing LDH level in the subject to the target LDH level.

115. The method of claim 114, wherein the target LDH level is a LDH level that is lower, relative to a control LDH level, by at least about 20%, 40%, 60%, or 80%.

116. The method of claim 115, wherein the control LDH level is a LDH level in a subject suffering from PNH and not receiving pegcetacoplan; a LDH level in the subject before receiving pegcetacoplan; or an upper limit of a range of LDH levels in a healthy subject.

117. The method of claim 114, wherein the target LDH level is about 110 to about 225 U/L, e.g., about 120, 140, 160, 180, 200, or 220 U/L.

118. The method of any one of claims 114–117, further comprising measuring LDH level in the subject.

119. The method of claim 118, comprising measuring LDH level in the subject before and/or after administration of pegcetacoplan.

120. The method of any one of claims 114–119, wherein LDH level is decreased in the subject in the absence of transfusion.

121. A method of treating a subject suffering from PNH, the method comprising subcutaneously administering to the subject about 1080 mg pegcetacoplan twice weekly or every three days, wherein the treatment decreases LDH level in the subject to a target LDH level.

122. The method of claim 121, wherein the target LDH level is a LDH level that is lower, relative to a control LDH level, by at least about 20%, 40%, 60%, or 80%.

123. The method of claim 122, wherein the control LDH level is a LDH level in a subject suffering from PNH and not receiving pegcetacoplan; a LDH level in the subject before receiving pegcetacoplan; or an upper limit of a range of LDH levels in a healthy subject.

124. The method of claim 121, wherein the target LDH level is about 110 to about 225 U/L, e.g., about 120, 140, 160, 180, 200, or 220 U/L.

125. The method of any one of claims 121–124, further comprising measuring LDH level in the subject.

126. The method of claim 125, comprising measuring LDH level in the subject before and/or after administration of pegcetacoplan.

127. The method of any one of claims 121–126, wherein the subject is treated in the absence of a transfusion.

128. A method of reducing fatigue level, in a subject suffering from PNH, to a target fatigue level, the method comprising subcutaneously administering to the subject about 1080 mg pegcetacoplan twice weekly or every three days, thereby reducing fatigue level in the subject to the target fatigue level.

129. The method of claim 128, wherein fatigue level is assessed using a FACIT-fatigue scale score.

130. The method of claim 129, wherein the target fatigue level is a FACIT-fatigue scale score that is higher, relative to a control FACIT-fatigue scale score, by at least 15, 10, 15, 20, or more points.

131. The method of claim 130, wherein the control FACIT-fatigue scale score is a FACIT-fatigue scale score from a subject suffering from PNH and not receiving pegcetacoplan; a FACIT-fatigue scale score from the subject before receiving pegcetacoplan; or a lower limit of a range of FACIT-fatigue scale scores from a healthy subject.

132. The method of claim 128, wherein the target fatigue level is a FACIT-fatigue scale score of about 32, 34, 36, 38, 40, 42, 44, 46, or 48.

133. The method of any one of claims 129–132, further comprising assessing FACIT-fatigue scale score from the subject.

134. The method of claim 133, comprising assessing FACIT-fatigue scale score from the subject before and/or after administration of pegcetacoplan.

135. The method of any one of claims 129–134, wherein FACIT-fatigue scale score from the subject is reduced in the absence of transfusion.

136. A method of treating a subject suffering from PNH, the method comprising subcutaneously administering to the subject about 1080 mg pegcetacoplan twice weekly or every three days, wherein the treatment reduces fatigue level in the subject to a target fatigue level.

137. The method of claim 136, wherein fatigue level is assessed using a FACIT-fatigue scale score.

138. The method of claim 137, wherein the target fatigue level is a FACIT-fatigue scale score that is higher, relative to a control FACIT-fatigue scale score, by at least 5, 10, 15, 20, or more points.

139. The method of claim 138, wherein the control FACIT-fatigue scale score is a FACIT-fatigue scale score from a subject suffering from PNH and not receiving pegcetacoplan; a FACIT-fatigue scale score from the subject before receiving pegcetacoplan; or a lower limit of a range of FACIT-fatigue scale scores from a healthy subject.

140. The method of claim 136, wherein the target fatigue level is a FACIT-fatigue scale score of about 32, 34, 36, 38, 40, 42, 44, 46, or 48.

141. The method of any one of claims 137–140, further comprising assessing FACIT-fatigue scale score from the subject.

142. The method of claim 141, comprising assessing FACIT-fatigue scale score from the subject before and/or after administration of pegcetacoplan.

143. The method of any one of claims 136–142, wherein the subject is treated in the absence of a transfusion.

144. The method of any one of the preceding claims, wherein prior to administration of pegcetacoplan, the subject has not received a C5 inhibitor, e.g., an anti-C5 antibody, e.g., eculizumab.

145. The method of any one of claims 60–143, wherein prior to administration of pegcetacoplan, the subject has received a C5 inhibitor, e.g., an anti-C5 antibody, e.g., eculizumab.

146. The method of claim 145, wherein the subject remains transfusion-dependent after receiving the C5 inhibitor, e.g., the anti-C5 antibody, e.g., eculizumab.

147. The method of claim 145 or 146, wherein the subject has a hemoglobin level of less than about 12 g/dL, e.g., less than about 11 g/dL, e.g., less than about 10.5 g/dL, e.g., less than about 10 g/dL, e.g., less than about 9 g/dL, e.g., less than about 8 g/dL after receiving the C5 inhibitor, e.g., the anti-C5 antibody, e.g., eculizumab.

148. The method of claim 144 or 145, wherein after administration of pegcetacoplan, the subject does not receive a dose of a C5 inhibitor, e.g., an anti-C5 antibody, e.g., eculizumab.

149. The method of claim 144 or 14586, wherein the subject receives at least one dose of pegcetacoplan in combination with at least one dose of a C5 inhibitor, e.g., an anti-C5 antibody, e.g., eculizumab.

150. The method of any one of the preceding claims, wherein pegcetacoplan is administered for at least about 12 weeks, about 16 weeks, about 20 weeks, about 24 weeks, about 28 weeks, about 32 weeks, about 36 weeks, about 40 weeks, about 44 weeks, about 48 weeks, or at least about 52 weeks.

151. The method of any one of the preceding claims, wherein about 1080 mg pegcetacoplan is self-administered by the subject twice weekly or every three days using a pump.

152. A method of treating a subject suffering from PNH, the method comprising subcutaneously administering to the subject about 1080 mg pegcetacoplan twice weekly or every three days.
